(19) United States
(12) Patent Application Publication
(10) Pub. No.: US 2025/0188160 A1
(43) Pub. Date: Jun. 12, 2025

(54) NOVEL ANGIOPOIETIN 2, VEGF DUAL ANTAGONISTS

(71) Applicant: AskGene Pharma Inc., Camarillo, CA (US)

(72) Inventors: Yuefeng LU, Newbury Park, CA (US); Jian-Feng LU, Oak Park, CA (US)

(21) Appl. No.: 18/782,004

(22) Filed: Jul. 23, 2024

Related U.S. Application Data
(63) Continuation of application No. 17/374,960, filed on Jul. 13, 2021, now Pat. No. 12,043,662.
Application No. 17/374,960 is a continuation of application No. 16/380,852, filed on Apr. 10, 2019, now Pat. No. 11,059,885.
Application No. 16/380,852 is a continuation-in-part of application No. 15/593,280, filed on May 11, 2017, now Pat. No. 10,654,922.
(60) Provisional application No. 62/655,436, filed on Apr. 10, 2018.
(60) Provisional application No. 62/336,522, filed on May 13, 2016.
(60) Provisional application No. 62/448,998, filed on Jan. 21, 2017.
(60) Provisional application No. 62/459,046, filed on Feb. 14, 2017.

(51) Int. Cl. C07K 16/22; A61K 9/08; A61K 9/19

(52) U.S. Cl. CPC C07K 16/22; A61K 9/08; A61K 9/19; C07K 2317/55; C07K 2317/622

(57) ABSTRACT

The present disclosure relates to fusion molecules and chimeric molecules which comprise two components: an Ang-2 antagonist peptide linked to a VEGF-binding moiety. Further disclosed are methods of using said chimeric molecules to treat a patient cancer, proliferative retinopathy, neovascular glaucoma, macular edema, wet age-related macular degeneration (wAMD), macular edema following retinal vein occlusion (RVO), diabetic macular edema (DME), or diabetic retinopathy (DR).

### CROSS-REFERENCE TO RELATED APPLICATIONS

The present application is a Continuation Application of U.S. patent application Ser. No. 16/380,852 which claims priority to U.S. Provisional Patent Application 62/336,552, filed May 13, 2016, Provisional Patent Application 62/459,046, filed Feb. 14, 2017 and Provisional Patent Application 62/448,998, filed Jan. 21, 2017, herein both incorporated by reference in their entirety. The present application also claims priority to U.S. Patent Application US20170327569A1 filed May 11, 2017, herein incorporated by reference in their entirety. The present application further claims priority to U.S. patent applications 62/655,436 filed on Apr. 10, 2018, herein incorporated by reference in their entirety.

### REFERENCE TO SEQUENCE LISTING

The entire content of the following electronic submission of the sequence listing via the USPTO EFS-WEB server, as authorized and set forth in MPEP § 1730 II.B.2(a)(C), is hereby expressly incorporated by reference in its entirety for all purposes. The sequence listing is identified on the electronically filed text file as follows: File Name: AG3-018US-SeqList_ST25; Date of Creation: Nov. 13, 2019; Size (bytes): 194 KB.

### FIELD OF THE INVENTION

The present application relates to novel molecules comprising binding domains to both VEGF and Ang2.

### INTRODUCTION

Angiogenesis is implicated in the pathogenesis of a variety of disorders including solid tumors, intraocular neovascular syndromes such as proliferative retinopathies or age-related macular degeneration (AMD), rheumatoid arthritis, and psoriasis (Folkman, J., et al., J. Biol. Chem. 267 (1992) 10931-10934; Klagsbrun, M., et al, Annu. Rev. Physiol. 53 (1991) 217-239; and Garner, A., Vascular Diseases, in: Pathobiology of Ocular Disease, A Dynamic Approach, Garner, A., and Klintworth, G. K. (eds.), 2nd edition, Marcel Dekker, New York (1994), pp. 1625-1710). In the case of solid tumors, the neovascularization allows the tumor cells to acquire a growth advantage and proliferative autonomy compared to the normal cells. Accordingly, a correlation has been observed between density of microvessels in tumor sections and patient survival in a number of cancers (see, e.g., Weidner, N., et al, N Engl J Med. 324 (1991) 1-8; Horak, E.R., et al, Lancet 340 (1992) 1120-1124; and Macchiarini, P., et al, Lancet 340 (1992) 145-146).

Human vascular endothelial growth factor (VEGF/VEGF-A) is described in, e.g., Leung, D.W., et al, Science 246 (1989) 1306-9; Keck, P.J., et al, Science 246 (1989) 1309-12 and Connolly, D.T., et al, J. Biol. Chem. 264 (1989) 20017-24. The expression of VEGF is potentiated in response to hypoxia, by activated oncogenes, and by a variety of cytokines. VEGF is involved in the regulation of normal and abnormal angiogenesis and neovascularization associated with tumors and intraocular disorders (Ferrara, N., et al, Endocr. Rev. 18 (1997) 4-25; Berkman, R.A., et al, J. Clin. Invest. 91 (1993) 153-159; Brown, L.F., et al, Human Pathol. 26 (1995) 86-91; Brown, L.F., et al, Cancer Res. 53 (1993) 4727-4735; Mattern, J., et al, Brit. J. Cancer. 73 (1996) 931-934; and Dvorak, H.F., et al, Am. J. Pathol. 146 (1995) 1029-1039).

Deregulated VEGF expression contributes to the development of solid tumors by promoting tumor angiogenesis and to the etiology of several additional diseases that are characterized by abnormal angiogenesis (Kim, K. J., et al., 1993. Nature (London) 362, 841-844; Millauer, B., et al., 1994. Nature (London) 367, 576-579). Consequently, inhibition of VEGF signaling abrogates the development of a wide variety of tumors.

In retinopathies, in which partial or general ischemia of the retina is accompanied by overexpression of VEGF and hyperproliferation of blood vessels, blindness can result (Aiello, L. P et al., 1994. N. Engl. J. Med. 331, 1480-1487; Adamis, A. P., et al.,, Am. J. Ophthalmol. 118, 445-450). Inhibition of VEGF expression in such disease states can treat or prevent resulting blindness.

Human angiopoietin-2 (ANG-2 or Ang-2 or Ang2) (alternatively abbreviated with ANGPT2 or ANG2) is described in Maisonpierre, P.C., et al, Science 277 (1997) 55-60 and Cheung, A.H., et al, Genomics 48 (1998) 389-91. Ang2 plays an important role in angiogenesis and its expression levels have been correlated with cancer and eye diseases (Gerald D et al., Cancer Res. 2013, 73(6):1649-57; Watanabe et al., Am J Ophthalmol. 2005, 139(3):476-81).

Dual antagonist RG7716 demonstrated superior efficacy than VEGF antagonist ranibizumab in a recent clinical trial. However, the reported dosage for RG7716 at 6 mg per dose was rather high considering the volume of administration to eye is typically low, e.g. 50 micoL). This could require a concentration of 120 mg/ml, a significant challenge for formulation development. A dual antagonist with stronger binding affinities to VEGF and/or Ang2 is needed. The present invention includes bi-specific molecules with enhanced binding ability and which result in a reduction in the severity of a disease in a patient treated with a molecule disclosed herein.

### SUMMARY

The present disclosure relates to novel bispecific chimeric molecules comprising binding domains to both VEGF and Ang-2. Further disclosed are methods of using said chimeric molecules to treat a patient of cancer, proliferative retinopathy, neovascular glaucoma, macular edema, wet age-related macular degeneration (wAMD), macular edema following retinal vein occlusion (RVO), diabetic macular edema (DME), or diabetic retinopathy (DR).

In some aspect, said chimeric molecule comprises one or two VEGF-binding moieties and one or two Ang-2 antagonist peptides, wherein:
a) said Ang-2 antagonist peptide comprises an amino acid sequence selected from SEQ ID NO: 8-14; and
b) said VEGF-binding moiety is an antibody, an Fab or an scFv; and wherein said antibody, Fab or scFv comprises light chain CDRs as derived from a light chain with an amino acid sequence as shown in SEQ ID NO: 4, or derived from a scFv with an amino acid sequence as shown in SEQ ID NO: 6, and heavy chain CDRs as derived from a heavy chain with an amino acid sequence as shown in SEQ ID NO: 5, or derived from a scFv with an amino acid sequence as shown in SEQ ID NO: 6.

In some embodiment, said VEGF binding moiety comprises an antibody with a light chain amino acid sequence that is at least 95% identical to that of SEQ ID NO: 4, and heavy chain amino acid sequence that is at least 99% identical to that of SEQ ID NO: 7.

In some embodiment, said Ang-2 antagonist peptide is fused to one or both of the N-terminals of the heavy chains (HC) of the said antibody optionally through a peptide linker. In some embodiment, the peptide-HC fusion polypeptide comprises an amino acid sequence that is at least 99% identical as one selected from SEQ ID NOS: 29, 30, and SEQ ID NO:33.

In some embodiment, said Ang-2 antagonist peptide is fused to one or both of the C-terminal of the heavy chain of the said antibody optionally through a peptide linker. In some embodiment, the Ang-2 antagonist peptide-heavy chain fusion polypeptide comprises an amino acid sequence that is at least 99% identical or 100% identical as one selected from SEQ ID NOS: 31, 32, and 34.

In some embodiment, said Ang-2 antagonist polypeptide is fused to the N-terminals or the C-terminals of the heavy chain of said antibody through a peptide linker; and wherein the Ang-2 antagonist peptide-heavy chain fusion polypeptide comprises an amino acid sequence at least 99% identical or 100% identical as one selected from SEQ ID NO: 37, 39, 41, 43, 45, 47, 49, 51, and 53.

In some embodiment, said VEGF binding moiety is an Fab with a light chain amino acid sequence of at least 95% identity to SEQ ID NO: 4, and a heavy chain amino acid sequence of at least 95% identity to SEQ ID NO: 5.

In some embodiment, th-e Ang2 antagonist peptide is fused to the N-terminal of the heavy chain of said Fab molecule through a peptide linker. In some embodiment, the Ang2 antagonist peptide-heavy chain fusion polypeptide has an amino acid sequence at least 99% identical as that of SEQ ID NO:19 or SEQ ID NO:20.

In some embodiment, the Ang-2 antagonist peptide is fused to the C-terminal of the heavy chain of said Fab molecule through a peptide linker. In some embodiment, the peptide-heavy chain fusion polypeptide has an amino acid sequence at least 99% identical as that of SEQ ID NO: 25 or SEQ ID NO:26.

In some embodiment, said VEGF binding moiety is an scFv with an amino acid sequence that has at least 95% identity to SEQ ID NO: 6. In some embodiment, the Ang-2 antagonist peptide is fused to the N-terminal of the scFv; and wherein the peptide-scFv fusion has an amino acid sequence selected from SEQ ID NOS: 21 and 22. In some embodiment the Ang-2 antagonist peptide is fused to the C-terminal of the scFv; and wherein the peptide-scFv fusion polypeptide has an amino acid sequence selected from SEQ ID NO:27 and SEQ ID NO:28.

In some aspect, said chimeric molecule comprises a fusion protein that has one or more VEGF-binding moieties and one or two Ang-2 antagonist peptides, wherein said VEGF binding moiety is a VEGF trap with an amino acid sequence having at least 95% identity to SEQ ID NO: 3; wherein said chimeric molecule comprises two identical polypeptide chains, which have an amino acid sequence at least 99% identical as one selected from SEQ ID NOS: 15-17, 23 and 24.

Also disclosed is a polynucleotide or polynucleotides encoding any one of the above said chimeric molecules. In some embodiment, said polynucleotide comprises a DNA sequence as one selected from SEQ ID NO: 35, 36, 38, 40, 42, 44, 46, 48, 50, 52, 54 and 56.

Also disclosed is an expression vector or vectors comprising the above said polynucleotide or polynucleotides.

Also disclosed is a host cell comprising the above said vector(s).

Also disclosed is a method of making any one of the above said chimeric molecules, comprising culturing the above said host cell under conditions that allow expression of the chimeric molecule, and isolating the chimeric molecule.

Also disclosed is a pharmaceutical composition comprising the chimeric molecule of any one of the above said chimeric molecule and a pharmaceutically acceptable excipient.

Further provide is a method of treating a patient with cancer, proliferative retinopathy, wet age-related macular degeneration (wAMD), macular edema following retinal vein occlusion (RVO), diabetic macular edema (DME), or diabetic retinopathy (DR) comprising administering to a subject of above said pharmaceutical composition.

### BRIEF DESCRIPTION OF THE DRAWINGS

FIG. 1. Protein A Affinity Chromatography. Approximately 150 ml of the clarified HEK 293 cell culture medium of the transient expression of AMD-B was loaded to a Protein A column (1×17 cm (Diameter×Height) of Captiv A Protein A resin) at 3 ml/min. The protein A column was equilibrated with an equilibration buffer (25 mM Tris Buffer, 100 mM NaCl, PH approximately 7.2). The column was washed with the Equilibration buffer and eluted with 2 M ariginine solution, PH 4.

FIG. 2. Kinetics of Ang-1 or Ang-2 Binding to AMD-E As Analyzed by Octet Red96.

FIG. 3. Binding of AMD A-E with VEGF.

FIG. 4A. Blocking of Binding of Ang-1 and Ang-2 to Tie-2 by AMD-A and AMD-B

FIG. 4B. Blocking of Binding of Ang-1 and Ang-2 to Tie-2 by AMD-C and AMD-D

FIG. 5. Blocking of Binding of Ang-2 to Tie-2 by ASKB712-O and ASKB712-O2

### DETAILED DESCRIPTION OF THE INVENTION

Disclosed herein are fusion proteins and chimeric molecules which comprise two components: an Ang-2 antagonist peptide operationally linked to a VEGF binding domain, which is selected from an anti-VEGF antibody, an anti-VEGF Fab, an anti-VEGF scFv, or a VEGF receptor extracellular domain-Fc fusion protein (or VEGF Trap). The Ang-2 antagonist peptide and VEGF binding domain are each defined below with reference to percent identity to a reference sequence. Further disclosed are methods of using said chimeric molecules to treat a patient of cancer, proliferative retinopathy, neovascular glaucoma, macular edema, wet age-related macular degeneration (wAMD), macular edema following retinal vein occlusion (RVO), diabetic macular edema (DME), or diabetic retinopathy (DR).

It is understood that aspects and variations of the invention described herein include “consisting” and/or “consisting essentially of” aspects and variations.

### Definitions

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody or antibody fragment, such as a monoclonal antibody, polyclonal, a chimeric antibody, a CDR-grafted antibody, a humanized antibody, a Fab, a Fab′, a F(ab′)₂, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies, a single chain antibody, and a Fc-containing polypeptide, such as an immunoadhesion. In some embodiments, the antibody may be of any heavy chain isotype (e.g., IgG, IgA, IgM, IgE, or IgD). In some embodiments, the antibody may be of any light chain isotype (e.g., kappa or gamma). The antibody may be non-human (e.g., from mouse, goat, or any other animal), fully human, humanized, or chimeric. In some embodiments, the antibody is a derivatized antibody.

The term “effective amount” used herein refers to an amount of a compound or composition sufficient to treat a specified disorder, condition, or disease, such as ameliorate, palliate, lessen, and/or delay one or more of its symptoms. In reference to a disease such as a cancer, an effective amount comprises an amount sufficient to cause a tumor to shrink and/or to decrease the growth rate of the tumor (such as to suppress tumor growth) or to prevent or delay other unwanted cell proliferation in the cancer. In some embodiments, the effective amount is an amount sufficient to delay development of a cancer. In some embodiments, the effective amount is an amount sufficient to prevent or delay recurrence. An effective amount can be administered in one or more administrations. In the case of a cancer, the effective amount of the drug or composition may: (i) reduce the number of epithelioid cells; (ii) reduce tumor size; (iii) inhibit, retard, slow to some extent and preferably stop the cancer cells infiltration into peripheral organs; (iv) inhibit (e.g., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “fused” or “fusion” in reference to two or more polypeptide sequences (such as an antibody heavy chain, antibody light chain, an antibody heavy chain fragment, an antibody light chain fragment, a drug conjugation moiety, a heterologous peptide, an albumin, or an albumin fragment) refers to joining of the polypeptide sequences through a backbone peptide bond.

The term “pharmaceutically acceptable” when used to refer to a compound or composition means that the compound or composition is suitable for administration to a subject, including a human subject, to achieve the treatments described herein, without unduly deleterious side effects in light of the severity of the disease and necessity of the treatment.

The term “subject” refers to a mammal and includes, but is not limited to, human, bovine, horse, feline, canine, rodent, or primate.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from a disease, diminishing the extent of a disease, stabilizing a disease (e.g., preventing or delaying the worsening of the disease), preventing or delaying the spread (e.g., metastasis) of a disease, preventing or delaying the recurrence of a disease, delaying or slowing the progression of a disease, ameliorating a disease state, providing remission (partial or total) of a disease, decreasing the dose of one or more other medications required to treat a disease, delaying the progression of a disease, increasing the quality of life, and/or prolonging survival. Also encompassed by “treatment” is a reduction of a pathological consequence of a disease (such as cancer). The methods of the invention contemplate any one or more of these aspects of treatment.

It is to be understood that one, some or all of the properties of the various embodiments described herein may be combined to form other embodiments of the present invention.

The section headings used herein are for organizational purposes only and are not to be construed as limiting the subject matter described.

### Ang-2 Antagonist Peptide

The fusion protein or chimeric molecule comprises an Ang-2 antagonist peptide component, which binds to Angiopoietin 2 (Ang-2) and inhibits the binding of Ang-2 to its receptor. One example of the peptide is called 2xCon4(C), as described in WO2004/092215A2 or WO03/05134A2. 2xCon4(C) has an amino acid sequence as shown in SEQ ID NO:1. Additional examples of Ang-2 binding peptides include but are not limited to: L-1-21, L1-7, L1-10, and L1-15, as described in WO2004/092215A2. Examples of Ang-2 antagonist peptides are shown in SEQ ID NO: 8-14.

### VEGF-Binding Moiety

The chimeric molecule further comprises a VEGF-binding moiety. In one embodiment, said VEGF-binding moiety is an anti-VEGF antibody, an anti-VEGF Fab, or an anti-VEGF scFv that inhibits the binding of VEGF to its receptors. One example of the VEGF antibody is bevacizumab, which has two heavy chains with amino acid sequence as shown as SEQ ID NO:1, and two light chains with amino acid sequence as shown as SEQ ID NO:2. Another example is ranibizumab, an anti-VEGF Fab. And a third example is Brolucizumab (RTH258), which is a humanized single-chain antibody fragment (scFv) against VEGF.

In another embodiment, said VEGF binding domain is a VEGF receptor-Fc fusion protein which “traps” VEGF (herein, referred to as a “VEGF trap”) and competes with the naturally occurring VEGF cellular receptor to inhibit VEGF. One example of the VEGF-receptor Fc fusion protein is afilbercept, which has an amino acid sequence as shown in SEQ ID NO:3.

In some embodiments, the VEGF-binding moiety comprises the six complementarity determining regions (CDRs) of Brolucizumab (RTH258), Ranibizumab or Bevacizumab. A number of CDR delineations are known in the art and are encompassed herein. A person of skill in the art can readily determine a CDR for a given delineation based on the sequence of the heavy or light chain variable region. The “Kabat” Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, Canonical structures for the hypervariable regions of immunoglobulins, J. Mol. Biol., vol. 196, pp. 901-917 (1987)). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid number of antibodies refers to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat | | | | |
| Numbering) | | | | |
| VH-CDR1 | H31-H35 | H26-H35 | H26-H32 | H30-H35 |
| (Chothia | | | | |
| Numbering) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

### Ang-2 Antagonist Peptide—VEGF-Binding Moiety Fusion Protein

The Ang-2 peptide can be linked or fused to either the C- or N-terminus of the VEGF antibody (e.g., either the heavy or the light chains) or the VEGF receptor-Fc fusion protein. The Fc portion of the VEGF receptor-Fc fusion protein may be located at either the C- or N-terminus of the VEGF receptor protein. The Fc portion is further defined herein.

The present compositions include “Fc fragments” or “Fc regions.” The term “Fc fragment” or “immunoglobulin Fc region” as used herein, refers to a protein that contains at least the heavy-chain constant region 2 (CH2) and the heavy-chain constant region 3 (CH3) of an immunoglobulin. In one embodiment, the Fc region excludes the variable regions of the heavy and light chains, the heavy-chain constant region 1 (CH1) and the light-chain constant region 1 (CL1) of the immunoglobulin. The Fc region may further include a hinge region at the heavy-chain constant region. Also, the immunoglobulin Fc region disclosed herein may contain a part or all of the Fc region including the heavy-chain constant region 1 (CH1) and/or the light-chain constant region 1 (CL1), except for the variable regions of the heavy and light chains, as long as it has a physiological function substantially similar to or better than the native protein. Also, the immunoglobulin Fc region may be a fragment having a deletion in a relatively long portion of the amino acid sequence of CH2 and/or CH3. That is, the immunoglobulin Fc region disclosed herein may comprise 1) a CH1 domain, a CH2 domain, a CH3 domain and a CH4 domain, 2) a CH1 domain and a CH2 domain, 3) a CH1 domain and a CH3 domain, 4) a CH2 domain and a CH3 domain, 5) a combination of one or more domains and an immunoglobulin hinge region (or a portion of the hinge region), and 6) a dimer of each domain of the heavy-chain constant regions and the light-chain constant region.

The immunoglobulin Fc region disclosed herein includes a native amino acid sequence, or a sequence analogue thereof. An amino acid sequence analogue is a sequence that is different from the native amino acid sequence due to a deletion, an insertion, a non-conservative or conservative substitution or combinations thereof of one or more amino acid residues.

Also, other various analogues are possible, including one in which a region capable of forming a disulfide bond is deleted, or certain amino acid residues are eliminated at the N-terminal end of a native Fc form or a methionine residue is added thereto. Further, to remove effector functions, a deletion may occur in a complement-binding site, such as a C1q-binding site and an ADCC (antibody dependent cell mediated cytotoxicity) site. Techniques of preparing such sequence analogues of the immunoglobulin Fc region are disclosed in WO 1997/034631 and WO 1996/032478.

The aforementioned Fc analogues are analogues that have a biological activity identical to the Fc region disclosed herein or improved structural stability, for example, against heat, pH, or the like.

In addition, these Fc regions may be obtained from native forms isolated from humans and other animals including cows, goats, pigs, mice, rabbits, hamsters, rats and guinea pigs, or may be recombinants or analogues thereof, obtained from transformed animal cells or microorganisms. Herein, they may be obtained from a native immunoglobulin by isolating whole immunoglobulins from human or animal organisms and treating them with a proteolytic enzyme. Papain digests the native immunoglobulin into Fab and Fc regions, and pepsin treatment results in the production of pF′c and F(ab)2 fragments. These fragments may be subjected, for example, to size exclusion chromatography to isolate Fc or pF′c. In another embodiment, a human-derived Fc region is a recombinant immunoglobulin Fc region that is obtained from a microorganism.

In one embodiment, the Fc region, if desired, may be modified by phosphorylation, sulfation, acrylation, glycosylation, methylation, farnesylation, acetylation, amidation, and the like. In one embodiment, the immunoglobulin Fc region disclosed herein may be in the form of having native sugar chains, increased sugar chains compared to a native form or decreased sugar chains compared to the native form, or may be in a deglycosylated form. The increase, decrease or removal of the immunoglobulin Fc sugar chains may be achieved by methods common in the art, such as a chemical method, an enzymatic method and a genetic engineering method using a microorganism. The removal of sugar chains from an Fc region results in a sharp decrease in binding affinity to the C1q part of the first complement component C1 and a decrease or loss in antibody-dependent cell-mediated cytotoxicity or complement-dependent cytotoxicity, thereby not inducing unnecessary immune responses in-vivo. In this regard, an immunoglobulin Fc region in a deglycosylated or aglycosylated form may be more suitable as a drug carrier.

As used herein, the term “deglycosylation” refers to enzymatically removing sugar moieties from an Fc region, and the term “aglycosylation” means that an Fc region is produced in an unglycosylated form by a prokaryote, preferably E. coli.

In one embodiment, the immunoglobulin Fc region may be an Fc region that is derived from IgG, IgA, IgD, IgE and IgM, or that is made by combinations thereof or hybrids thereof. In an embodiment, it is derived from IgG or IgM, which are among the most abundant proteins in human blood, and further, wherein an IgG, which is known to enhance the half-lives of ligand-binding proteins is an IgG1, IgG2a, IgG2b and/or IgG3.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Methods for obtaining (e.g., producing, isolating, purifying, synthesizing, and recombinantly manufacturing) polypeptides are well known to one of ordinary skill in the art.

### Pharmaceutical Compositions

Pharmaceutical compositions of the chimeric molecules are prepared by mixing the antibody fusion molecules or the antibody fusion molecule drug conjugate having the desired degree of purity with one or more optional pharmaceutically acceptable carriers (see Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may comprise as—or in addition to—the carrier, excipient or diluent any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be administered by a number of routes. In some embodiment, said formulation is administrated directly in a tumor or tumors.

In an embodiment, a host cell is a cell that is transfected with an expression vector containing a nucleotide or polynucleotide sequence that encodes one or more protein sequences that can be expressed in a cell. In an embodiment, a cell, including a host cell is a mammalian cell, a yeast cell, an insect cell, or a bacteria. In a further embodiment, a mammalian cell used as a host cell can be a Chinese hamster ovary (“CHO”) cell, a HeLa cell, an HEK cell, including an HEK-293 cell. In another embodiment, a yeast cell used as a host cell can be S. cerevisiae or Pichia pastoris. In an embodiment, an insect cell used as a host cell can be Sf9, Sf21, Hi-5, Schneider 2 cells, Schneider 3 cells or High Five. In a further embodiment, a bacterial cell used as a host cell can be E. coli, Corynebacterium or C. glutamicum.

In some embodiments, an antibody or protein formulation is a lyophilized formulation. In another embodiment, an antibody or protein formulation is an aqueous formulation.

In other aspects of this embodiment, a fusion protein or chimeric molecule disclosed herein reduces the severity of a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a fusion protein or chimeric molecule disclosed herein reduces the severity of a disease from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A fusion protein or chimeric molecule disclosed herein may comprise a therapeutic compound in an amount sufficient to allow customary administration to an individual and with other excipients may constitute a pharmaceutical composition. In aspects of this embodiment, a therapeutic compound disclosed herein may be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a therapeutic compound. In other aspects of this embodiment, a therapeutic compound disclosed herein may be, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a therapeutic compound. In yet other aspects of this embodiment, a therapeutic compound disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg. In still other aspects of this embodiment, a therapeutic compound disclosed herein may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

A therapeutic compound disclosed herein may comprise a solvent, emulsion or other diluent in an amount sufficient to dissolve a therapeutic compound disclosed herein. In other aspects of this embodiment, a therapeutic compound disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a therapeutic compound disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a therapeutic compound disclosed herein in a pharmaceutical composition disclosed herein may be of any concentration desired. In an aspect of this embodiment, the final concentration of a therapeutic compound in a pharmaceutical composition may be a therapeutically effective amount. In other aspects of this embodiment, the final concentration of a therapeutic compound in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of a therapeutic compound disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL. In other aspects of this embodiment, the final concentration of a therapeutic compound in a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/ml to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/ml to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual suffering from a disease, including a cancer. As used herein, the term “treating,” refers to reducing or eliminating in an individual a clinical symptom of cancer; or delaying or preventing in an individual the onset of a clinical symptom of a disease, including a cancer. For example, the term “treating” can mean reducing a symptom of a condition characterized by a cancer, including, but not limited to, tumor size, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with cancer are well known and can be determined by a person of ordinary skill in the art by taking into account factors, including, without limitation, the location of the disease, including a cancer, the cause of the disease, including a cancer, the severity of the disease, including a cancer, and/or the tissue or organ affected by the disease, including a cancer. Those of skill in the art will know the appropriate symptoms or indicators associated with a specific type of disease, including a cancer and will know how to determine if an individual is a candidate for treatment as disclosed herein.

In aspects of this embodiment, a therapeutically effective amount of a therapeutic compound disclosed herein reduces a symptom associated with a disease, including a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic compound disclosed herein reduces a symptom associated with a disease, including a cancer by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic compound disclosed herein reduces a symptom associated with a disease, including a cancer by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of a therapeutic compound disclosed herein generally is in the range of about 0.001 mg/kg/day to about 100 mg/kg/day. In aspects of this embodiment, an effective amount of a therapeutic compound disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In other aspects of this embodiment, an effective amount of a therapeutic compound disclosed herein may be in the range of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day. In yet other aspects of this embodiment, an effective amount of a therapeutic compound disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day. In still other aspects of this embodiment, an effective amount of a therapeutic compound disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

In other aspects of this embodiment, an effective amount of a therapeutic compound disclosed herein may be in the range of, e.g., about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day. In yet other aspects of this embodiment, an effective amount of a therapeutic compound disclosed herein may be in the range of, e.g., about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day.

In liquid and semi-solid formulations, a concentration of a therapeutic compound disclosed herein typically may be between about 50 mg/mL to about 1,000 mg/mL. In aspects of this embodiment, a therapeutically effective amount of a therapeutic compound disclosed herein may be from, e.g., about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/ml to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

Dosing can be single dosage or cumulative (serial dosing), and can be readily determined by one skilled in the art. For instance, treatment of a disease, including a cancer may comprise a one-time administration of an effective dose of a therapeutic compound or a pharmaceutical composition disclosed herein. Alternatively, treatment of a disease, including a cancer may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a therapeutic compound or pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a therapeutic compound or pharmaceutical composition disclosed herein that is administered can be adjusted accordingly.

In one embodiment, a therapeutic compound disclosed herein is capable of reducing the number of cancer cells or tumor size in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic compound is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a therapeutic compound and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a therapeutic compound is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects, of this embodiment, a therapeutically effective amount of a therapeutic compound disclosed herein reduces or maintains a disease, including a cancer cell population and/or tumor cell size in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic compound disclosed herein reduces or maintains a disease or a cancer cell population and/or tumor cell size in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic compound disclosed herein reduces or maintains a disease, including a cancer cell population and/or tumor cell size in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A pharmaceutical composition or therapeutic compound is administered to an individual. An individual is typically a human being, but can be an animal, including, but not limited to, dogs, cats, birds, cattle, horses, sheep, goats, reptiles and other animals, whether domesticated or not. Typically, any individual who is a candidate for treatment is a candidate with some form of disease, including a cancer, whether the cancer is benign or malignant, a tumor, solid or otherwise, a cancer cell not located in a tumor or some other form of cancer. Among the most common types of cancer include, but are not limited to, bladder cancer, breast cancer, colon and rectal cancer, endometrial cancer, kidney cancer, renal cancer, leukemia, lung cancer, melanoma, non-Hodgkins lymphoma, pancreatic cancer, prostate cancer, stomach cancer and thyroid cancer. Pre-operative evaluation typically includes routine history and physical examination in addition to thorough informed consent disclosing all relevant risks and benefits of the procedure.

### EXAMPLES

### Example 1—Production of the Chimeric Molecule Comprising VEGF Antibody and Ang-2 Binding Peptide in HEK293 Cells

Chimeric molecules named AMD A, B, C, D and E (see Table 2) were expressed through transient expression by HEK-293 cells. Briefly, DNAs (SEQ ID NOs: 58, 59, 60 and 63) for the fusion proteins comprising VEGF antibody light chain with or without Ang2 binding peptides and DNAs (SEQ ID NOs: 57, 61 and 62) for the fusion proteins comprising VEGF antibody heavy chain with Ang2 binding peptides were synthesized and cloned into expression vectors._The complete expression constructs comprising the genes were confirmed by DNA sequencing. DNA constructs were transformed into E. coli DH5alfa competent cells (Invitrogen). Single clone was selected and cultured in LB broth with antibiotics (kanamycin, 25 ug/mL). DNA plasmids were extracted with Qiagen Plasmid Maxi Kit (Qiagen) following manufacture's protocol. Plasmid concentration was measured by NanoDrop (Thermo Fisher). The expression plasmid constructs containing the DNA sequences encoding the genes of interest, were introduced into HEK-293 cells transiently by using polyethylenimine (PEI). The transfected cells were treated by alproic acid (VPA) 24 hours post transfection to enhance protein expression.

TABLE 2
AMD Molecules

| | AMD-A | AMD-B | AMD-C | AMD-D | AMD-E |
|---|---|---|---|---|---|
| Light | Peptide | Bevacizumab | Peptide L1-15 | Bevacizumab | Bevacizumab |
| Chain | L1-15 (No | light chain | (with LE) | light chain | light chain |
| | LE*) fused to | | fused to | | |
| | N-terminus of | | N-terminus of | | |
| | Bevacizumab | | Bevacizumab | | |
| | light chain | | light chain | | |
| Light | SEQ ID | SEQ ID | SEQ ID | SEQ ID | SEQ ID NO |
| Chain | NO: | NO: | NO: 60 | NO: 63 | 58 |
| DNA | 59 | 63 | (LY2.55.2) | (LY2.55.5) | (DHAMDL |
| SEQ | (LY2.55.1) | (LY2.55.5) | | | 083016) |
| ID NO | | | | | |
| Heavy | Peptide | Peptide | Peptide | Peptide | Peptide |
| Chain | L1-15 (No | L1-15 (No | L1-15 (with | L1-15 (with | 2xCon4(C) |
| | LE) fused to | LE) fused to | LE) fused to | LE) fused to | fused to the C- |
| | N-terminus of | N-terminus of | N-terminus of | N-terminus of | terminus of |
| | Bevacizumab | Bevacizumab | Bevacizumab | Bevacizumab | the Heavy |
| | heavy chain | heavy chain | heavy chain | heavy chain | Chain of |
| | | | | | Bevacizumab |
| Heavy | SEQ ID | SEQ ID | SEQ ID | SEQ ID | SEQ ID NO |
| Chain | NO: 61 | NO: 61 | NO: 62 | NO: 62 | 57 |
| DNA | (LY2.55.3) | (LY2.55.3) | (LY2.55.4) | (LY2.55.4) | (DHAMDH |
| SEQ | | | | | 02083016) |
| ID NO | | | | | |

* LE is one of the flanking sequences, which were present both in the original phage clone when the peptides were screened and in the subsequent peptibody (Peptide-Fc fusion) molecules.

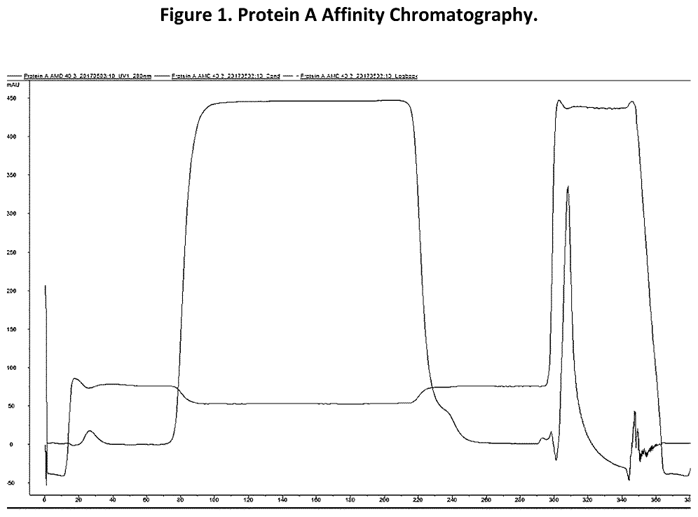

After approximately 6 days of culturing, the cell culture media were harvested by clarifying centrifugation at 9000 rpm for 30-60 minutes followed by filtration through 0.22 micrometer filters. The clarified supernants were loaded to a Protein A affinity column and the chimeric molecules (AMD-A, B, C, D and E) were purified. The chimeric molecules were eluted using 2 M arginine solution, pH 4 from the protein A column. FIG. 1 shows a representative chromatograph of the Protein A column step. Table 3 summarizes the results from the purification of the chimeric molecules. As shown in Table 3, chimeric molecules containing a total of 2 copies L1-15 peptides (AMD-B and AMD-D), both fused to the N-terminals of the heavy chain, had significantly higher expression levels comparing to the ones with a total of four copies of L1-15 peptides (AMD-A and AMD-C), wherein there is one each of L1-15 peptide fused to the N-terminals of both the light chains and the heavy chains of the antibody. With or without the flanking sequence LE as part of the L1-15 peptide did not appear to affect the expression of the chimeric molecules.

The expression level of AMD-E was comparable to that of AMD-B and AMD-D (Table 3). AMD-E has one Peptide 2xCon4(C) fused to each of the C-terminus of the heavy chains of Bevacizumab. The purity of the products were analyzed using SDS electrophoresis and/or HPLC methods.

TABLE 3
Summary of Protein A Affinity Chromatography Purification

| | AMD-A | AMD-B | AMD-C | AMD-D | AMD-E |
|---|---|---|---|---|---|
| Approximate | 150 | 200 | 150 | 150 | 200 |
| culture | | | | | |
| volume | | | | | |
| (ml) | | | | | |
| Protein A | 3 | 9 | 5 | 12 | 17 |
| Pool | | | | | |
| Volume | | | | | |
| (ml) | | | | | |
| OD280 | 0.24 | 1.14 | 0.29 | 0.88 | 0.80 |
| Approximate | 0.45 | 6.4 | 0.91 | 6.6 | 8.5 |
| amount in | | | | | |
| the | | | | | |
| Protein A | | | | | |
| Pool, | | | | | |
| (mg) | | | | | |

### Example 2—Production of the Chimeric Molecule Comprising VEGF Trap and Ang2 Binding Peptide in CHO Cells

DNA for the chimeric molecule comprising the VEGF Receptor-Fc fusion protein (VEGF Trap) and the Ang-2 binding peptide (SEQ ID NO: 64, named as ASKB-E06) is synthesized and cloned into an expression vector. The complete expression construct comprising the DNA gene is confirmed by DNA sequencing. The expression construct is amplified by transforming into DH10B E. coli and culturing the cells overnight. DNA for the expression construct was prepared and purified by endo-free plasmid kit (from QIAGEN®).

Cell lines stably expressing ASKB-E06 is obtained by transfecting the expression construct into GS^{-/-} Chinese hamster ovarian cells (CHO) by electroporation and screening for transfected CHO cells using a selective culture medium without glutamine (EX-CELL® CD CHO Fusion Growth Medium). In this manner 32 or more stable minipools are established and the leading mini-pool is selected based on expression level in batch and fed-batch cultures. The expression levels are detected by ELISA titer assay. Single cloning is performed by limited dilution and using clone media, two leading single clones out of more than100 positive clones are selected based on productivity and cell growth in batch and fed-batch culture. The lead clones are expanded and seeded at 0.5×10⁶ cells/mL, total 300 mL in 2L shake flasks, and the cells are cultured at 37° C., 5% CO₂, 70% HMR conditions and shaking at 120 rpm. The cultures are fed by using 5% Acti CHO® Feed A +0.5% Feed B (from GE Health) on Day 3, 6, 7, 8 and 9. The cell viability, viable cell density are monitored every other day, the cultures are harvested on Day 11-13.

The cell culture medium is harvested by clarifying approximately 600 ml of the cultured cell medium through centrifugation at 2000 rpm for 10 minutes followed by filtration. The clarified supernant is loaded to a Protein A affinity column and the chimeric molecule is purified. The protein is further purified using ion exchange chromatography, hydrophobic interaction chromatography, hydroxyapatite chromatography, and/or mixed mode chromatography. The product is further concentrated and buffer exchanged using UFDF and further formulated. The purity of the product is analyzed using CE-SDS and HPLC methods.

### Example 3—Molecular Assays to Evaluate Dual Antagonist Activities of the Chimeric Molecules

Molecular assays (Octet Binding Affinity, Affinity ELISA, and Blocking ELISA) were developed to assess direct binding of the chimeric molecules to ANG-1, Ang-2 and/or VEGF, and the effect of the chimeric molecules on the Ang1:Tie-2 interaction, Ang-2:Tie-2 interaction and/or VEGF: VEGF receptor interaction. These in vitro assays are described as the following:

### Octet Affinity

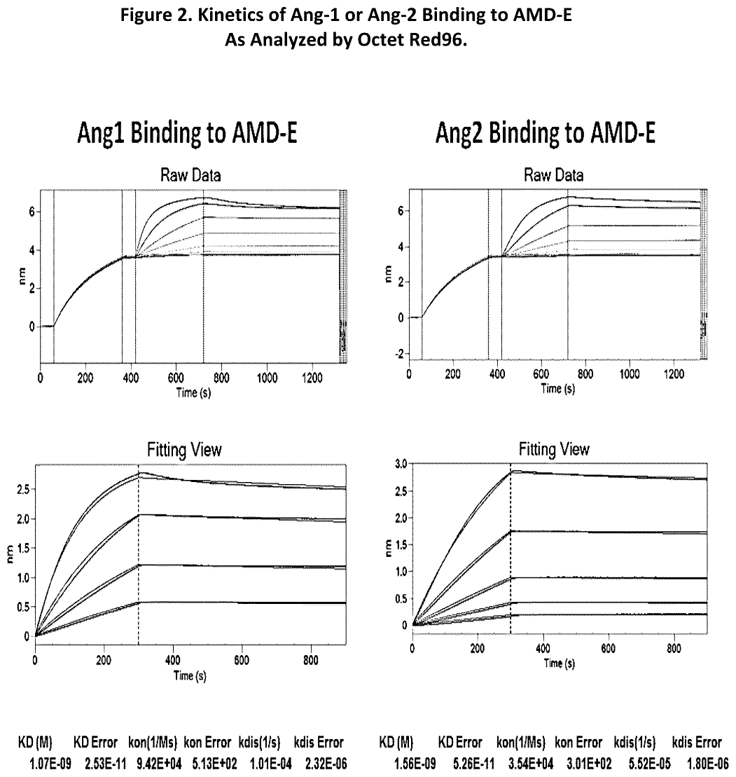

Purified recombinant human VEGF protein was ordered from Life-Technologies (Cat. #PHC9391). Human Ang1 or Ang2 protein were ordered from R&D System. Analysis was carried out using Octet Red96 from Pall ForteBio. Using anti-human IgG Fc sensors, a sample of chimeric molecule AMD-B, AMD-D, AMD-E or the control antibody Bevacizumab was loaded for 300 seconds at 3 ug/mL in the kinetics buffer. Ligands ANG1, ANG2, or VEGF samples were associated for 300 seconds using a dilution series starting at 5 or 10 ug/mL and sequentially diluting 2-fold for 7 wells. Dissociation was run for 600 seconds. Data was analyzed using a 1:1 model with global fit. A representative binding kinetics graph is shown in FIG. 2. The binding affinity results are summarized in Tables 4A, 4B and 4C. The results showed that the chimeric molecules AMD-B, AMD-D, and AMD-E were able to bind to Ang1, Ang2, and VEGF. It was also noticed that the chimeric molecule AMD-B with four L1-15 peptides fused to the N-terminals of the antibody had reduced affinity to VEGF when comparing to the control antibody Bevacizumab. AMD-D and AMD-E showed comparable affinity to VEGF comparing to the control antibody ASKB1202, a biosimilar m An internal control, ASKB 1202, a biosimilar to Bevacizumab developed in-house.

TABLE 4A
Summary of the Octet Affinity analysis results-Binding
of ANG-1.

| | kon | | |
|---|---|---|---|
| | (1/Ms) | koff (1/s) | KD (M) |
| AMD-B | 1.56E+05 | 2.69E−04 | 1.73E−09 |
| AMD-D | 1.75E+05 | 2.41E−04 | 1.37E−09 |
| AMD-E | 9.42E+04 | 1.01E−04 | 1.07E−09 |

TABLE 4B
Summary of the Octet Affinity analysis results-Binding
of ANG-2.

| | kon | | |
|---|---|---|---|
| | (1/Ms) | koff (1/s) | KD (M) |
| AMD-B | 3.34E+04 | 4.33E−05 | 1.30E−09 |
| AMD-D | 3.68E+04 | 2.39E−05 | 6.49E−10 |
| AMD-E | 3.54E+04 | 5.52E−05 | 1.56E−09 |

TABLE 4C
Summary of the Octet Affinity analysis results-Binding
of VEGF.

| | kon | | |
|---|---|---|---|
| | (1/Ms) | koff (1/s) | KD (M) |
| Bevacizumab | 8.03E+04 | <1.0E−07 | <1.0E−12 |
| AMD-B | 1.41E+05 | 3.42E−05 | 2.42E−10 |
| AMD-D | 1.01E+05 | <1.0E−07 | <1.0E−12 |
| AMD-E | 1.38E+05 | <1.0E−07 | <1.0E−12 |

Affinity ELISA: Purified recombinant human VEGF protein was ordered from Life-Technologies (Cat. #PHC9391). VEGF is reconstituted in BSA solution at 0.1 mg/mL as recommended by the manufacturer. Aliquots the samples were made and stored at −20° C.

Using microtiter plates, approximately 100 microliters per well of VEGF is added to each well and the plates were incubated about 2 hours, after which the plates are washed with phosphate buffered saline (PBS) containing about 0.1 percent Tween-20 four times. The wells are then blocked using about 250 microliters per well of about 5 percent BSA in PBS, and the plates were incubated at room temperature for about 2 hours. After incubation, excess blocking solution is discarded, and about 100 microliters of AMD-A, B, C, D or E was added to a well in a dilution series starting at a concentration of about 40 nanomolar and then serially diluting 4-fold in PBS containing about 1 percent BSA. The plates were then incubated overnight at room temperature. After incubation, plates were washed with PBS containing about 0.1 percent Tween-20. Washing was repeated four additional times, after which about 100 microliters per well of goat anti-human IgG (Fc)-HRP (Pierce Chemical Co., catalog #31416) previously diluted 1:5000 in PBS containing 1 percent BSA was added. Plates were incubated approximately 1 hour at room temperature. Plates were then washed five times in PBS containing about 0.1 percent Tween-20, after which about 100 microliters per well of TMB (3,3′,5,5′-Tetramethylbenzidine Liquid Substrate System; Sigma Chemical Company, St. Louis, MO, catalog number T8665) substrate was added and plates are incubated about 5-15 minutes until blue color developed. Absorbance was then read in a spectrophotometer at about 450 nm.

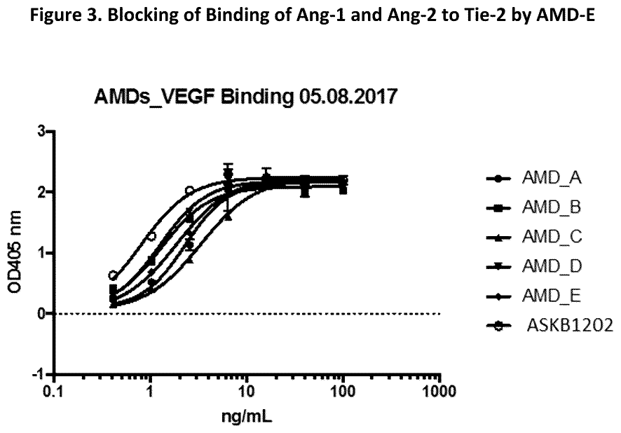

FIG. 3 shows the ELISA results of binding of VEGF to AMD-A, B, C, D, and E. An internal control, ASKB 1202, a biosimilar to Bevacizumab currently in development, was used as a positive control. The results showed that all the molecules AMD-A, B, C, D and E retained abilities to bind to VEGF. The EC-50 results are summarized in Table 5. The results showed that the AMD-B and AMD-D had VEGF binding affinity close to ASKB1202. In addition, AMD-B and AMD-D had stronger VEGF binding affinity than AMD-A and AMD-C.

TABLE 5
Affinity ELISA Results: Binding of VEGF to AMD-A, B,
C, D and E.

| | EC-50 (ng/ml) |
|---|---|
| AMD-A | 2.296 |
| AMD-B | 1.278 |
| AMD-C | 3.328 |
| AMD-D | 1.247 |
| AMD-E | 1.87 |
| ASKB1202 | 0.8002 |

### Blocking ELISA

The chimeric molecules were assessed in their abilities in blocking the binding of Ang1 and Ang2 to their receptor Tie-2. 96 well microtiter plate (Nunk) was coated with 100 uL final concentration 100 ng/mL of human Tie2-Fc (R&D System, 313-T1) diluted in 0.1 M carbonate (pH9.3) at 4° C. overnight. The plate was then blocked for 2 hours with 5% BSA in PBST (0.05% Tween 20). Purified chimeric molecule, at starting concentration of 1000 ng/ml, was serially diluted with dilution factor of three in PBS with 1% BSA. Human Ang1 or Ang2 protein (R&D System) was added to final concentration of 50 ng/ml and incubated at room temperature for 1 hour. The Chimeric molecule-Ang1 or Chimeric molecule-Ang2 mixture was then added into microtiter plate coated with human Tie2-Fc and incubate for another 1 hour at room temperature. 100 uL anti-Ang1 or anti-Ang2 monoclonal antibody (R&D System) was added into each well at final concentration of 1 ug/mL and incubated for 1 hour at room temperature. Horseradish-peroxidase (HRP) conjugated anti-mouse IgG secondary antibody was added at 1:5000 dilution and incubated for 1 hour at room temperature. Standard colorimetric response was developed by using TMB (Pierce). Absorbance was read at OD450by spectrophotometer. Between each step, the plate was washed 5 time with 100 uL PBS.

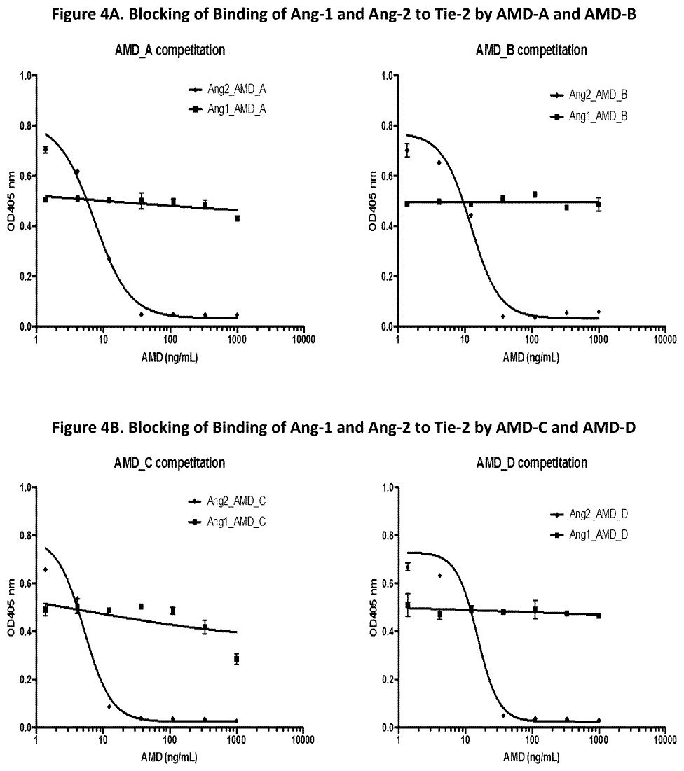

The dose dependent inhibition or lack of inhibition of the binding of Ang1 and Ang-2 to receptor Tie-2 are shown in FIG. 4. The IC-50 results are summarized in Table 6. The results showed that the chimeric molecules AMD-A, B, C, and D selectively inhibited the binding of Ang2 to Tie-2, with IC-50 in the range of 5-15 ng/ml; while their abilities in inhibiting the binding of Ang-1 to Tie-2 were very weak, if any, despite the fact that they all were able to bind to Ang1. The results also showed that AMD-A and AMD-C, both comprising 4 copies of the peptide L1-15 had lower IC-50 than AMD-B and AMD-D. AMD-E was able to inhibit the association of both Ang-1 and Ang-2 to their receptor Tie-2.

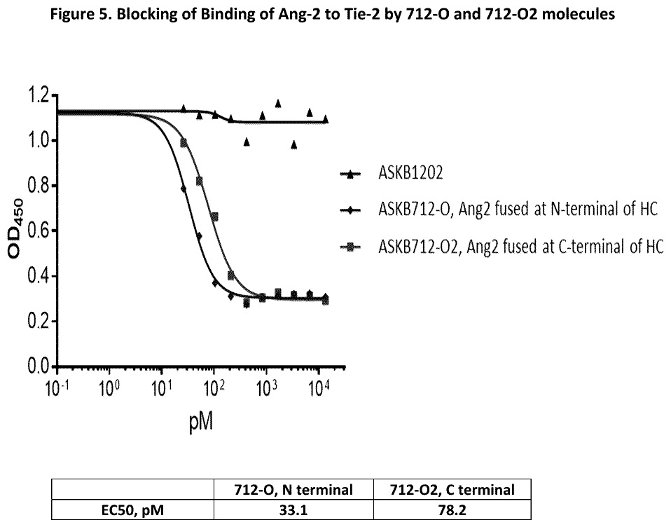

FIG. 5 shows the inhibition of the binding of Ang-2 to Tie-2 by chimeric molecules 712-O and 712-O2. The chimeric molecule 712-O comprises two heavy chain polypeptide chains with an amino acid sequence as shown in SEQ ID NO: 29 and two light chains with an amino acid sequence as shown in SEQ ID NO: 4. The chimeric molecule 712-O2 comprises two heavy chain polypeptide chains with an amino acid sequence as shown in SEQ ID NO: 31and two light chains with an amino acid sequence as shown in SEQ ID NO: 4.

The Ang-2 antagonist peptide L1-15 is fused to the N-terminals of the heavy chains of a VEGF-binding antibody in the case of 712-O. In the case of 712-O2, L1-15 is fused to the C-terminals of the heavy chains. The IC-50′s for the Ang-2 blocking assay were approximately 33 PM for 712-O and approximately 78 pM for 712-O2. Since L1-15, together with other peptides including L1-7, L1-10 and L1-21, was considered an N-terminal fusion peptide and was only tested to be active when it is fused to the N-terminal of the Fc as described in WO2004/092215A2. It was surprised that the chimeric molecule 712-O2 was significantly potent with an IC-50 of approximately 78 pM.

TABLE 6
Blocking ELISA Results: Inhibition of Binding of Ang-1 or
Ang-2 to Tie-2.

| | | IC-50 of Inhibiting |
|---|---|---|
| | IC-50 of inhibiting Ang- | Ang-2 Binding |
| | 1 Binding (ng/ml) | (ng/ml) |
| AMD-A | Not detected | 7.3 |
| AMD-B | Not detected | 12.49 |
| AMD-C | Not detected | 5.136 |
| AMD-D | Not detected | 15.21 |
| AMD-E | 10 (estimated) | 2.107 |

### Example 4—Cell-Based Activity Assay: In Vitro Human Umbilical Vein Endothelial Cells (HUVEC) Tube-Formation Assay

In order to confirm whether or not ASKB-E06 inhibits angiogenesis, proliferation, migration, and differentiation assays of human umbilical vein endothelial cells (HUVEC) are performed.

### a) Proliferation Inhibition of HUVEC by 712-O

After 10,000 HUVEC were added to 100 μl of EBM-2 medium (Lonza, Switzerland), EBM-2 medium having VEGF-A (50 ng/ml) is added thereto, or EBM-2 medium including VEGF-A (50 ng/ml) and 712-O sample at different concentration is added thereto in each well of a 96-well plate, followed by incubation under 5% CO₂, at 37° C. for 72 hours. Then, 10 μl of WST-1 solution was added thereto, followed by incubation at 37° C. for 4 hours. Absorbance is measured at 410 nm with a reference of 610 nm. The results are shown in Table 7, which indicated that 712-O had similar or higher potency than Lucentis®. It was more potent than ASKB1202 (a biosimilar of bevacizumab).

TABLE 7
HUVEC Assay Results

| | Approximate MW | |
|---|---|---|
| | (KD) | EC50 (nM) |
| 712-0 (Lot# LL21- | 156 | 0.57 |
| 05) | | |
| ASKB1202 (Lot# | 149 | 1.28 |
| DS20150403) | | |
| Lutenis ® | 48 | 0.72 |

### (2) Migration Inhibition of HUVEC by 712-O

After a bottom of Transwells, (Corning Inc., US) having a pore size of 8-μm is coated with 0.1% gelatin and mounted in a 24-well plate, a lower chamber is filled with 600 μl of EBM-2 medium (Lonza), EBM-2 with VEGF-A (50 ng/ml), or EBM-2 with VEGF-A (50 ng/ml) and 712-O sample at different concentration. An upper chamber is provided with 100 μl of EBM-2 medium containing 1×10⁵ HUVEC. After incubation in 37° C. cell incubator for 4 hours, a filter is detached from the Transwell and cells are fixed with methanol for 1 minute and stained with Hematoxylin/Eosin. Cells which do not migrate but are left on an upper surface of the transwell are completely removed with a cotton swab. Five random fields among the cells migrated through the filter are arbitrarily chosen under an optical microscope (×100) and the number thereof is counted.

### (3) Inhibition of Tube Formation by 712-O

In order to confirm that ASKB-E06 can inhibit differentiation of HUVEC, tube formation assay is performed. More specifically, after a 96-well plate is coated with Growth Factor Reduced Matrigel (BD Biosciences, US), 15,000 HUVEC in 100 μl of EBM-2 medium, EBM-2 medium with VEGF-A (50 ng/ml), or EBM-2 medium with VEGF-A (50 ng/ml) and an antibody sample are added to each well, followed by incubation in 37° C. cell incubator for 6 hours. Then, tube formation is observed by using an inverted microscope.

### Example 5—In Vivo Anti-Tumor Activity Study: Therapeutic Efficacy Studies With Systemically Administered Dual Antagonist Chimeric Molecules

The chimeric molecule ASKB712-B is administered subcutaneously to A431 tumor-bearing mice at a once-per-day schedule 72 hours after tumor challenge. The doses used are 1000, 200, 40 and 8 ug/mouse/day. A total of 20 doses is given to all animals. Tumor volumes and body weights are recorded three times/week. At the end of the study, animals are sacrificed, and their sera are collected for measuring ASKB712-B levels by ELISA. Tumors and a panel of normal tissues are collected from all groups.

The non-limiting examples provided herein are for illustrative purposes only in order to facilitate a more complete understanding of the disclosed subject matter. These examples should not be construed to limit any of the embodiments described in the present specification, including those pertaining to the fusion peptides, pharmaceutical compositions, or methods and uses for treating cancer, proliferative retinopathies, AMD or RA.

A chimeric molecule, which comprises one or two VEGF-binding moieties and one or two Ang-2 antagonist peptides, wherein:
a) said Ang-2 antagonist peptide comprises an amino acid sequence selected from SEQ ID NO: 8-14; and
b) said VEGF-binding moiety is an antibody, an Fab or an scFv; and wherein said antibody, Fab or scFv comprises light chain CDRs as derived from a light chain with an amino acid sequence as shown in SEQ ID NO: 4, or derived from a scFv with an amino acid sequence as shown in SEQ ID NO: 6, and heavy chain CDRs as derived from a heavy chain with an amino acid sequence as shown in SEQ ID NO: 5, or derived from a scFv with an amino acid sequence as shown in SEQ ID NO: 6.

The chimeric molecule of claim 1, wherein said VEGF binding moiety comprises an antibody with a light chain amino acid sequence that is at least 95% identical to that of SEQ ID NO: 4, and heavy chain amino acid sequence that is at least 99% identical to that of SEQ ID NO: 7.

The chimeric molecule of claims 2, wherein said Ang-2 antagonist peptide is fused to the N-terminal of the heavy chain (HC) of the said antibody optionally through a peptide linker.

The chimeric molecule of claim 3, wherein the Ang-2 antagonist peptide-HC fusion polypeptide comprises an amino acid sequence that has at least 99% identity to one of SEQ ID NOS: 29, 30, and SEQ ID NO:33.

The chimeric molecule of claim 2, wherein said Ang-2 antagonist peptide is fused to the C-terminal of the heavy chain of the said antibody optionally through a peptide linker.

The chimeric molecule of claim 5, wherein the Ang-2 antagonist peptide-heavy chain fusion polypeptide comprises an amino acid sequence at least 99% identical or 100% identical as one selected from SEQ ID NOS: 31, 32, and 34.

The chimeric molecule of claim 2, wherein said Ang-2 antagonist polypeptide is fused to the N-terminals or the C-terminals of the heavy chain of said antibody through a peptide linker; and wherein the Ang-2 antagonist peptide-heavy chain fusion polypeptide comprises an amino acid sequence at least 99% identical or 100% identical as one selected from SEQ ID NO: 37, 39, 41, 43, 45, 47, 49, 51, and 53.

The chimeric molecule of claim 1, wherein said VEGF binding moiety is an Fab with a light chain amino acid sequence of at least 95% identity to SEQ ID NO: 4, and a heavy chain amino acid sequence of at least 95% identity to SEQ ID NO: 5.

The chimeric molecule of claim 8, wherein the Ang-2 antagonist peptide is fused to the N-terminal of the heavy chain of said Fab molecule through a peptide linker.

The chimeric molecule of claim 9, wherein the Ang-2 antagonist peptide-heavy chain fusion polypeptide has an amino acid sequence at least 99% identical as that of SEQ ID NO:19 or SEQ ID NO:20.

The chimeric molecule of claim 8, wherein the Ang-2 antagonist peptide is fused to the C-terminal of the heavy chain of said Fab molecule through a peptide linker.

Chimeric molecule of claim 11, wherein the Ang-2 antagonist peptide-heavy chain fusion polypeptide has an amino acid sequence at least 99% identical to SEQ ID NO: 25 or SEQ ID NO:26.

The chimeric molecule of claim 1, wherein said VEGF binding moiety is an scFv with an amino acid sequence having at least 95% identity to SEQ ID NO: 6.

The chimeric molecule of claim 13, wherein the Ang-2 antagonist peptide is fused to the N-terminal of the scFv; and wherein the peptide-scFv fusion has an amino acid sequence selected from SEQ ID NOS:21 and 22.

The chimeric molecule of claim 13, wherein the Ang2 antagonist peptide is fused to the C-terminal of the scFv optionally; and wherein the peptide-scFv fusion has an amino acid sequence selected from SEQ ID NO:27 and SEQ ID NO:28.

A chimeric molecule comprising a fusion protein that has one or more VEGF-binding moieties and one or two Ang-2 antagonist peptides, wherein said VEGF binding moiety is a VEGF trap with an amino acid sequence having at least 95% identity to SEQ ID NO: 3; wherein the chimeric molecule comprises two identical polypeptide chains, each having an amino acid sequence at least 99% identical to one of SEQ ID NOS: 15-17, 23 and 24.

A polynucleotide or polynucleotides encoding the chimeric molecule of any one of claims 1-16.

An expression vector or vectors containing a polynucleotide or polynucleotides of claim 17.

A host cell transfected with one or more of the expression vectors of claim 18.

A method of making the chimeric molecule of any one of claims 1-16, comprising culturing a host cell transfected with one or more expression vectors containing a polynucleotide that encodes a chimeric molecule of one of claims 1-16 under conditions that allow expression of the chimeric molecule, and isolating the chimeric molecule.

A pharmaceutical composition comprising the chimeric molecule of any one of claims 1-16 and a pharmaceutically acceptable excipient.

The pharmaceutical composition of claim 21, wherein the pharmaceutical composition contains one or more acceptable carriers.

The pharmaceutical composition of claim 21, wherein the pharmaceutical composition is in the form of a lyophilized formulation or an aqueous solution.

The pharmaceutical compositions of claim 21, wherein the pharmaceutical composition includes one or more of carriers, an excipient, a diluent, a suitable binder, a lubricant, a suspension agent, a coating agent or a solubilizing agent.

A method of treating a patient with cancer, proliferative retinopathy, wet age-related macular degeneration (wAMD), macular edema following retinal vein occlusion (RVO), diabetic macular edema (DME), or diabetic retinopathy (DR) comprising administering to a subject a pharmaceutical composition of claim 21.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only and is not intended to limit the scope of the present invention, which is defined solely by the claims. Accordingly, the present invention is not limited to that precisely as shown and described.

```
SEQUENCES
SEQ ID NO: 1, Bevacizumab Heavy Chain:
10 20 30 40 50 60
EVQLVESGGG LVQPGGSLRL SCAASGYTFT NYGMNWVRQA PGKGLEWVGW INTYTGEPTY
        70 80 90 100 110 120
AADFKRRFTF SLDTSKSTAY LGMNSLRAED TAVYYCAKYP HYYGSSHWYF DVWGQGTLVT
      130 140 150 160 170 180
VSSASTKGPS VFPLAPSSKS TSGGTAALGC LVKDYFPEPV TVSWNSGALT SGVHTFPAVL
      190 200 210 220 230 240
QSSGLYSLSS VVTVPSSSLG TQTYICNVNH KPSNTKVDKK VEPKSCDKTH TCPPCPAPEL
      250 260 270 260 290 300
LGGPSVFLFP PKPKDTLMIS RTPEVTCVVV DVSHEDPEVK FNWYVDGVEV HNAKTKPREE
      310 320 330 340 350 360
QYNSTYRVVS VLTVLHQDWL NGKEYKCKVS NKALPAPIEK TISKAKGQPR EPQVYTLPPS
      370 380 390 400 410 420
REEMTKNQVS LTCLVKGFYP SDIAVEWESN GQPENNYKTT PPVLDSDGSF FLYSKLTVDK
      430 440 450
SRWQQGNVFS CSVMHEALHN HYTQKSLSLS PGK
SEQ ID NO: 2, Bevacizumab Light Chain:
10 20 30 40 50 60
DIQMTQSPSS LSASVGDRVT ITCSASQDIS NYLNWYQQKP GKAPKVLIYF TSSLHSGVPS
      70 80 90 100 110 120
RFSGSGSGTD FTLTISSLQP EDFATYYCQQ YSTVPWTFGQ GTKVEIKRTV AAPSYFIFPP
      130 140 150 160 170 180
SDEQLKSGTA SVVCLLNNFY PREAKVQWKV DNALQSGNSQ ESVTEQDSKD STYSLSSTLT
      190 200 210
LSKADYEKHK VYACEVTHQ6 LSSPVTKSFN RGEC
SEQ ID NO: 3, VEGF Trap Aflibercept
10 20 30 40 50 60
SDTGRPFVEM YSEIPEIIHM TEGRELVIPC RVTSPNITVT LKKFPLDTLI PDGKRIIWDS
        70 80 90 100 110 120
RKGFIISNAT YKEIGLLTCE ATVNGHLYKT NYLTHRQTNT IIDVVLSPSH GIELSVGEKL
      130 140 150 160 170 180
VLNCTARTEL NVGIDFNWEY PSSKHQHKKL VNRDLKTQSG SEMKKFLSTL TIDGVTRSDQ
      190 200 210 220 230 240
GLYTCAASSG LMTKKNSTFV RVHEKDKTHT CPPCPAPELL GGPSVFLFPP KPKDTLMISR
      250 260 270 260 290 300
TPEVTCVVVD VSHEDPEVKF NWYVDGVEVH NAKTKPREEQ YNSTYRVVSV LTVLHQDWLN
      310 320 330 340 350 360
GKEYKGKVSN KALPApTeKT ISKAKGGPRE PQVYTLPPSR DELTKNQVSL TCLVKGFYPS
      370 380 390 400 410 420
DIAVEWESNG GPENNYKTTP PVLDSDGSFF LYSKLTVDKS RWQQGNVFSC SVMHEALHNH
        430
YTGKSLSLSP G(K)
SEQ ID NO: 4, Protein Sequence for Light Chain,
Ranibizumab (VEGF Fab)
DIQLTQSPSSLSASVGDRVTITCSASQDISNYLNWYQQKPGKAPKVLIYFTSSLHSGVPS
RFSGSGSGTDFTLTISSLQPEDFATYYCQQYSTVPWTFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO: 5, Protein Sequence for Heavy Chain
Ranibizumab (VEGF Fab)
EVQLVESGGGLVQPGGSLRLSCAASGYDFTHYGMNWVRQAPGKGLEWVGWINTYTGE
PTYAADFKRRFTFSLDTSKSTAYLQMNSLRAEDTAVYYCAKYPYYYGTSHWYFDVWGQG
TLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHL
SEQ ID NO: 6, Protein Sequence for a VEGF ScFv
EIVMTQSPSTLSASVGDRVIITCQASEIIHSWLAWYQQKPGKAPKLLIYLASTLASGV
PSRFSGSGSGAEFTLTISSLQPDDFATYYCQNVYLASTNGANFGQGTKLTVLGGGGGSG
GGGSGGGGSGGGGSEVQLVESGGGLVQPGGSLRLSCTASGFSLTDYYYMTWVRQAPGK
GLEWVGFIDPDDDPYYATWAKGRFTISRDNSKNTLYLQMNSLRAEDTAVYYCAGGDHN
SGWGLDIWGQGTLVTVSS
SEQ ID NO: 7, Protein Sequence for a heavy chain
of a VEGF antibody
EVQLVESGGGLVQPGGSLRLSCAASGYDFTHYGMNWVRQAPGKGLEWWVGWINTYTGE
PTYAADFKRRFTFSLDTSKSTAYLQMNSLRAEDTAVYYCAKYPYYYGTSHWYFDVWGQ
GTLVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 8, L1-7
AQQTNFMPM DDLEQRLYEQ FILQQG
SEQ ID NO: 9, L1-10
AQQKFQPLD ELEQTLYEQF MLQQA
SEQ ID: NO: 10, L1-15
AQQKYQPLD ELDKTLYDQF MLQQG
SEQ ID NO: 11, L1-7B
QTNFMPM DDLEQRLYEQ FILQQG
SEQ ID NO: 12, L1-10B
QKFQPLD ELEQTLYEQF MLQQA
SEQ ID NO: 13, L1-15B
QKYQPLD ELDKTLYDQF MLQQG
SEQ ID NO: 14, CVX-060:
QKYQPLDEKDKTLYDQFMLQQG
SEQ ID NO 15, L1-15 fused to the N-terminus of the VEGF Trap,
with linker peptide GGGGSGGGGSGGGGS
      10 20 30 40 50 60
AQQKYQPLDE LDKTLYDQFM LQQGGGGGSG GGGSGGGGSS DTGRPFVEMY SEIPEIIHMT
      70 80 90 100 110 120
EGRELVIPCR VTSPNITVTL KKFPLDTLIP DGKRIIWDSR KGFIISNATY KEIGLLTCEA
      130 140 150 160 170 180
TVNGHLYKTN YLTHRQTNTI IDVVLSPSHG IELSVGEKLV LNCTARTELN VGIDFNWEYP
      190 200 210 220 230 240
SSKHQHKKLV NRDLKTQSGS EMKKFLSTLT IDGVTRSDQG LYTCAASSGL MTKKNSTFVR
      250 260 270 260 290 300
VHEKDKTHTC PPCPAPELLG GPSVFLFPPK PKDTLMISRT PEVTCVVVDV SHEDPEVKFN
      310 320 330 340 350 360
WYVDGVEVHN AKTKPREEQY NSTYRVVSVL TVLHQDWLNG KEYKCKVSNK ALPAPIEKTI
      370 380 390 400 410 420
SKAKGQPREP QVYTLPPSRD ELTKNQVSLT CLVKGFYPSD IAVEWESNGQ PENNYKTTPP
      430 440 450 460 470
VLDSDGSFFL YSKLTVDKSR WQQGNVFSCS VMHEALHNHY TQKSLSLSPG K
SEQ ID NO 16, Protein Sequence for AMD-I (L1-15 fused to VEGF Trap)
Xaa₁Xaa₂QKXaa₅QPLDELXaa₁₂Xaa₁₃TLYXaa₁₇QFMLQQGXaa₂₅Xaa₂₆
(GGGGS)ₙSDTGRPFVEMYSEIPEIIHMTEGRELVIPCRVTSPNITVTLKKFPLD
TLIPDGKRIIWDSRKGFIISNATYKEIGLLTCEATVNGHLYKTNYLTHRQTNTII
DVVLSPSHGIELSVGEKLVLNCTARTELNVGIDFNWEYPSSKHQHKKLVNRDLKT
QSGSEMKKFLSTLTIDGVTRSDQGLYTCAASSGLMTKKNSTFVRVHEKDKTHTCP
PCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPG
(K)
wherein Xaa1 is A, G, or deleted;
Xaa2 is Q or A or deleted;
Xaa25 is L or deleted;
Xaa5 is Y
or F;
Xaa12 is D or E;
Xaa13 is Q or K;
Xaa17 is D or E;
Xaa26 is E is deleted; n = 0, 1,
2, 3, 4, or 5; and
the C-terminal amino acid K may be deleted.
SEQ ID NO 17, Protein Sequence for AMD-J (L1-7 fused
to VEGF Trap)
Xaa₁Xaa₂QTNFMPMDDLEQRLYEQFILQQGXaa₂₆Xaa₂₇(GGGGS)ₙSDTGRP
FVEMYSEIPEIIHMTEGRELVIPCRVTSPNITVTLKKFPLDTLIPDGKRIIWDSR
KGFIISNATYKEIGLLTCEATVNGHLYKTNYLTHRQTNTIIDVVLSPSHGIELSV
GEKLVLNCTARTELNVGIDFNWEYPSSKHQHKKLVNRDLKTQSGSEMKKFLSTLT
IDGVTRSDQGLYTCAASSGLMTKKNSTFVRVHEKDKTHTCPPCPAPELLGGPSVF
LFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPG(K)
wherein Xaa1 is A, G, or deleted;
Xaa2 is Q or A or deleted;
Xaa26 is L or deleted;
Xaa27 is E is deleted; n = 0, 1, 2, 3, 4, or 5;
and the C-terminal amino acid K may be deleted.
SEQ ID NO 19, Protein Sequence for AMD-K Heavy Chain
(L1-15 fused to VEGF Fab)
Xaa₁Xaa₂QKXaa₅QPLDELXaa₁₂Xaa₁₃TLYXaa₁₇QFMLQQGXaa₂₅Xaa₂₆
(GGGGS)ₙEVQLVESGGGLVQPGGSLRLSCAASGYDFTHYGMNWVRQAPGKGLE
WVGWINTYTGEPTYAADFKRRFTFSLDTSKSTAYLQMNSLRAEDTAVYYCAKYP
YYYGTSHWYFDVWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVN
HKPSNTKVDKKVEPKSCDKTHL
wherein Xaa1 is A, G, or deleted;
Xaa2 is Q or A or deleted;
Xaa5 is Y or F;
Xaa12 is D or E;
Xaa13 is Q or K;
Xaa17 is D or E;
Xaa25 is L or deleted;
Xaa26 is E is deleted; and n = 0,
1, 2, 3, 4, or 5.
SEQ ID NO 20, Protein Sequence for AMD-L Heavy Chain
(L1-7 fused to VEGF Fab)
Xaa₁Xaa₂QTNFMPMDDLEQRLYEQFILQQGXaa₂₆Xaa₂₇(GGGGS)ₙEVQLVE
SGGGLVQPGGSLRLSCAASGYDFTHYGMNWVRQAPGKGLEWVGWINTYTGEPT
YAADFKRRFTFSLDTSKSTAYLQMNSLRAEDTAVYYCAKYPYYYGTSHWYFDVW
GQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEP
KSCDKTHL
wherein Xaa1 is A, G, or deleted;
Xaa2 is Q or A or deleted;
Xaa26 is L or deleted;
Xaa27 is E is deleted;
and n = 0, 1, 2, 3, 4, or 5.
SEQ ID NO 21, Protein Sequence for AMD-N (L1-15 fused
to VEGF ScFv)
Xaa₁Xaa₂QKXaa₅QPLDELXaa₁₂Xaa₁₃TLYXaa₁₇QFMLQQGXaa₂₅Xaa₂₆
(GGGGS)ₙEIVMTQSPSTLSASVGDRVIITCQASEIIHSWLAWYQQKPGKAPKLLI
YLASTLASGVPSRFSGSGSGAEFTLTISSLQPDDFATYYCQNVYLASTNGANFGQ
GTKLTVLGGGGGSGGGGSGGGGSGGGGSEVQLVESGGGLVQPGGSLRLSCTASGF
SLTDYYYMTWVRQAPGKGLEWVGFIDPDDDPYYATWAKGRFTISRDNSKNTLY
LQMNSLRAEDTAVYYCAGGDHNSGWGLDIWGQGTLVTVSS
wherein Xaa1 is A, G, or deleted;
Xaa2 is Q or A or deleted;
Xaa5 is Y or F;
Xaa12 is D or E;
Xaa13 is Q or K;
Xaa17 is D or E;
Xaa25 is L or deleted;
Xaa26 is E is deleted; and
n = 0, 1, 2, 3, 4, or 5
SEQ ID NO 22, Protein Sequence for AMD-Q (L1-7 fused
to VEGF ScFv)
Xaa₁Xaa₂QTNFMPMDDLEQRLYEQFILQQGXaa₂₆Xaa₂₇(GGGGS)ₙEIVMTQ
SPSTLSASVGDRVIITCQASEIIHSWLAWYQQKPGKAPKLLIYLASTLASGVPSRF
SGSGSGAEFTLTISSLQPDDFATYYCQNVYLASTNGANFGQGTKLTVLGGGGGSG
GGGSGGGGSGGGGSEVQLVESGGGLVQPGGSLRLSCTASGFSLTDYYYMTWVRQ
APGKGLEWVGFIDPDDDPYYATWAKGRFTISRDNSKNTLYLQMNSLRAEDTAVY
YCAGGDHNSGWGLDIWGQGTLVTVSS
wherein Xaa1 is A, G, or deleted;
Xaa2 is Q or A or deleted;
Xaa26 is L or deleted;
Xaa27 is
E is deleted; and n = 0, 1, 2, 3, 4, or 5.
SEQ ID NO 23, Protein Sequence for AMD-I-C terminal
(L1-15 fused to C-terminal of VEGF Trap)
SDTGRPFVEMYSEIPEIIHMTEGRELVIPCRVTSPNITVTLKKFPLDTLIPDGKR
IIWDSRKGFIISNATYKEIGLLTCEATVNGHLYKTNYLTHRQTNTIIDVVLSPSH
GIELSVGEKLVLNCTARTELNVGIDFNWEYPSSKHQHKKLVNRDLKTQSGSEMKK
FLSTLTIDGVTRSDQGLYTCAASSGLMTKKNSTFVRVHEKDKTHTCPPCPAPELL
GGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGA(GGGGS)ₙ
Xaa₁Xaa₂QKXaa₅QPLDELXaa₁₂Xaa₁₃TLYXaa₁₇QFMLQQGXaa₂₅Xaa₂₆
wherein Xaa1 is A, G, or deleted;
Xaa2 is Q or A or deleted;
Xaa5 is Y or F;
Xaa12 is D or E;
Xaa13 is Q or K;
Xaa17 is D or E;
Xaa25 is L or deleted;
Xaa26 is E is deleted; and
n = 0, 1, 2, 3, 4, or 5.
SEQ ID NO 24, Protein Sequence for AMD-J-C terminal
(L1-7 fused to C-terminal of VEGF Trap)
SDTGRPFVEMYSEIPEIIHMTEGRELVIPCRVTSPNITVTLKKFPLDTLIPDGKR
IIWDSRKGFIISNATYKEIGLLTCEATVNGHLYKTNYLTHRQTNTIIDVVLSPSH
GIELSVGEKLVLNCTARTELNVGIDFNWEYPSSKHQHKKLVNRDLKTQSGSEMKK
FLSTLTIDGVTRSDQGLYTCAASSGLMTKKNSTFVRVHEKDKTHTCPPCPAPELL
GGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGA
(GGGGS)ₙXaa₁Xaa₂QTNFMPMDDLEQRLYEQFILQQGXaa₂₆Xaa₂₇
wherein Xaa1 is A, G, or deleted;
Xaa2 is Q or A or deleted;
Xaa26 is L or deleted;
Xaa27 is E is deleted;
and n = 0, 1, 2, 3, 4, or 5.
SEQ ID NO 25, Protein Sequence for AMD-K-C terminal
Heavy Chain (L1-15 fused to C-terminal VEGF Fab)
EVQLVESGGGLVQPGGSLRLSCAASGYDFTHYGMNWVRQAPGKGLEWVGWINT
YTGEPTYAADFKRRFTFSLDTSKSTAYLQMNSLRAEDTAVYYCAKYPYYYGTSHW
YFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHL(GGGGS)ₙXaa₁Xaa₂QKXaa₅QPLDELXaa₁₂Xaa₁₃TLY
Xaa₁₇QFMLQQGXaa₂₅Xaa₂₆
wherein Xaa1 is A, G, or deleted;
Xaa2 is Q or A or deleted;
Xaa5 is Y or F;
Xaa12 is D or E;
Xaa13 is Q or K;
Xaa17 is D or E;
Xaa25 is L or deleted;
Xaa26 is E is deleted;
and n = 0, 1, 2, 3, 4, or 5.
SEQ ID NO 26, Protein Sequence for AMD-L-C terminal
Heavy Chain (L1-7 fused to VEGF Fab)
EVQLVESGGGLVQPGGSLRLSCAASGYDFTHYGMNWVRQAPGKGLEWVGWINT
YTGEPTYAADFKRRFTFSLDTSKSTAYLQMNSLRAEDTAVYYCAKYPYYYGTSHV
VYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHL(GGGGS)ₙXaa1Xaa2QTNFMPMDDLEQRLYEQFILQQG
Xaa₂₆Xaa₂₇
wherein Xaa1 is A, G, or deleted;
Xaa2 is Q or A or deleted;
Xaa26 is L or deleted;
Xaa27 is
E is deleted; and n = 0, 1, 2, 3, 4, or 5.
SEQ ID NO 27, Protein Sequence for AMD-N-C terminal
(L1-15 fused to C-terminal VEGF ScFv)
EIVMTQSPSTLSASVGDRVIITCQASEIIHSWLAWYQQKPGKAPKLLIYLASTLAS
GVPSRFSGSGSGAEFTLTISSLQPDDFATYYCQNVYLASTNGANFGQGTKLTVLG
GGGGSGGGGSGGGGSGGGGSEVQLVESGGGLVQPGGSLRLSCTASGFSLTDYYYM
TWVRQAPGKGLEWVGFIDPDDDPYYATWAKGRFTISRDNSKNTLYLQMNSLRA
EDTAVYYCAGGDHNSGWGLDIWGQGTLVTVSS(GGGGS)ₙXaa₁Xaa₂QKXaa₅QP
LDELXaa₁₂Xaa₁₃TLYXaa₁₇QFMLQQGXaa₂₅Xaa₂₆;
wherein Xaa1 is A, G, or deleted;
Xaa2 is Q or A or deleted;
Xaa5 is Y
or F;
Xaa12 is D or E;
Xaa13 is Q or K;
Xaa 17 is D or E;
Xaa25 is L or deleted;
Xaa26 is
E is deleted; and n = 0, 1, 2, 3, 4, or 5.
SEQ ID NO 28, Protein Sequence for AMD-Q-C terminal
(L1-7 fused to VEGF ScFv)
EIVMTQSPSTLSASVGDRVIITCQASEIIHSWLAWYQQKPGKAPKLLIYLASTLAS
GVPSRFSGSGSGAEFTLTISSLQPDDFATYYCQNVYLASTNGANFGQGTKLTVLG
GGGGSGGGGSGGGGSGGGGSEVQLVESGGGLVQPGGSLRLSCTASGFSLTDYYYM
TWVRQAPGKGLEWVGFIDPDDDPYYATWAKGRFTISRDNSKNTLYLQMNSLRA
EDTAVYYCAGGDHNSGWGLDIWGQGTLVTVSS(GGGGS)nXaa1Xaa2QTNFMPM
DDLEQRLYEQFILQQGXaa26Xaa27
wherein Xaa1 is A, G, or deleted;
Xaa2 is Q or A or deleted;
Xaa26 is L or deleted;
Xaa27 is E is deleted;
and n = 0, 1, 2, 3, 4, or 5.
SEQ ID NO: 29, Protein Sequence for ASKB712-O (L1-15
fused to the N-terminal of an VEGF-binding antibody)
AQQKYQPLDELDKTLYDQFMLQQGLEGGGGSGGGGSGGGGSEVQLVESGGGLVQP
GGSLRLSCAASGYDFTHYGMNWVRQAPGKGLEWVGWINTYTGEPTYAADFKRR
FTFSLDTSKSTAYLQMNSLRAEDTAVYYCAKYPYYYGTSHWYFDVWGQGTLVTV
SSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHT
CPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLS
PGK
SEQ ID NO: 30, Protein Sequence for ASKB712-O3
(L1-15 fused to the N-terminal of an VEGF-binding
antibody)
Xaa₁Xaa₂QKXaa₅QPLDELXaa₁₂Xaa₁₃TLYXaa₁₇QFMLQQGXaa₂₅Xaa₂₆
(GGGGS)ₙEVQLVESGGGLVQPGGSLRLSCAASGYDFTHYGMNWVRQAPGKGLE
WVGWINTYTGEPTYAADFKRRFTFSLDTSKSTAYLQMNSLRAEDTAVYYCAKYP
YYYGTSHWYFDVWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNV
FSCSVMHEALHNHYTQKSLSLSPGK;
wherein Xaa1 is A, G, or deleted;
Xaa2 is Q or A or deleted;
Xaa5 is Y or F;
Xaa12 is D or E;
Xaa13 is Q or K;
Xaa17 is D or E;
Xaa25 is L or deleted;
Xaa26 is E is deleted;
n = 0, 1, 2, 3, 4, or 5;
and the C-terminal amino acid K may be deleted.
SEQ ID NO: 31, Protein Sequence for ASKB712-O2
(L1-15 fused to the C-terminal of an VEGF-binding antibody)
EVQLVESGGGLVQPGGSLRLSCAASGYDFTHYGMNWVRQAPGKGLEWVGWINT
YTGEPTYAADFKRRFTFSLDTSKSTAYLQMNSLRAEDTAVYYCAKYPYYYGTSHV
VYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHE
ALHNHYTQKSLSLSPGAGGGGSGGGGSGGGGSAQQKYQPLDELDKTLYDQFMLQQ
GLE
SEQ ID NO: 32, Protein Sequence for ASKB712-O4
(L1-15 fused to the C-terminal of an
VEGF-binding antibody)
EVQLVESGGGLVQPGGSLRLSCAASGYDFTHYGMNWVRQAPGKGLEWVGWINT
YTGEPTYAADFKRRFTFSLDTSKSTAYLQMNSLRAEDTAVYYCAKYPYYYGTSHV
VYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHE
ALHNHYTQKSLSLSPGA(GGGGS)ₙXaa₁Xaa₂QKXaa₅QPLDELXaa₁₂Xaa₁₃T
LYXaa₁₇QFMLQQGXaa₂₅Xaa₂₆
wherein Xaa1 is A, G, or deleted;
Xaa2 is Q or A or deleted;
Xaa25 is L or deleted;
Xaa5 is Y or F;
Xaa12 is D or E;
Xaa13 is Q or K;
Xaa17 is D or E;
Xaa26 is E is deleted;
n = 0, 1, 2, 3, 4, or 5.
SEQ ID NO: 33, Protein Sequence for ASKB712-P (L1-7
fused to the N-terminal of an VEGF-binding antibody)
Xaa1Xaa2QTNFMPMDDLEQRLYEQFILQQGXaa26Xaa27(GGGGS)nEVQLVE
SGGGLVQPGGSLRLSCAASGYDFTHYGMNWVRQAPGKGLEWVGWINTYTGEPT
YAADFKRRFTFSLDTSKSTAYLQMNSLRAEDTAVYYCAKYPYYYGTSHWYFDVW
GQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHY
TQKSLSLSPGK
wherein Xaa1 is A, G, or deleted;
Xaa2 is Q or A or deleted;
Xaa26 is L or deleted;
Xaa27 is
E is deleted; and n = 0, 1, 2, 3, 4, or 5.
SEQ ID NO: 34, Protein Sequence for ASKB712-P2 (L1-7
fused to the C-terminal of an VEGF-binding antibody)
EVQLVESGGGLVQPGGSLRLSCAASGYDFTHYGMNWVRQAPGKGLEWVGWINT
YTGEPTYAADFKRRFTFSLDTSKSTAYLQMNSLRAEDTAVYYCAKYPYYYGTSHW
YFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHE
ALHNHYTQKSLSLSPGA(GGGGS)nXaa1Xaa2QTNFMPMDDLEQRLYEQFILQQ
GXaa26Xaa27
wherein Xaa1 is A, G, or deleted;
Xaa2 is Q or A or deleted;
Xaa26 is L or deleted;
Xaa27 is
E is deleted;
SEQ ID NO: 35-712O_L7_3xGS_DNA sequence
ATGGAATTCGGCCTGTCTTGGCTGTTCCTGGTGGCCATTCTGAAGGGCGCTCTGG
CCGCTCAGACCAACTTCATGCCTATGGACGACCTGGAACAGCGGCTGTACGAGCA
GTTCATCCTGCAGCAAGGACTGGAAGGCGGCGGAGGATCTGGCGGAGGCGGTAGC
GGAGGCGGTGGATCTGAAGTGCAGCTGGTTGAAAGTGGCGGCGGATTGGTTCAGC
CTGGCGGATCTCTGAGACTGTCTTGTGCCGCCTCTGGCTACGACTTCACCCACTA
CGGCATGAATTGGGTCCGACAGGCTCCCGGCAAAGGCCTGGAATGGGTCGGATGG
ATCAACACCTATACCGGCGAGCCTACCTACGCCGCCGATTTCAAGCGGAGATTCA
CCTTCTCCCTGGACACCTCCAAGTCTACCGCCTACCTGCAGATGAACTCCCTGAG
AGCCGAGGACACCGCCGTGTACTACTGCGCTAAGTACCCCTACTACTACGGCACC
AGCCACTGGTACTTCGACGTGTGGGGACAGGGCACACTGGTCACAGTGTCCTCCG
CCTCTACCAAGGGACCCTCTGTGTTTCCTCTGGCTCCCTCCAGCAAGTCCACCTC
TGGTGGAACAGCTGCTCTGGGCTGCCTGGTCAAGGACTACTTTCCTGAGCCTGTG
ACCGTGTCCTGGGCTTCTGGTGCTCTGACATCTGGCGTGCACACCTTTCCAGCTG
TGCTGCAGTCCTCCGGCCTGTACTCTCTGTCCTCTGTCGTGACCGTGCCTTCCAG
CTCTCTGGGAACCCAGACCTACATCTGCAATGTGAACCACAAGCCTTCCAACACC
AAGGTCGACAAGAAGGTGGAACCCAAGTCCTGCGATAAGACCCACACCTGTCCTC
CATGTCCTGCTCCAGAAGCTGCTGGCGGCCCATCCGTGTTTCTGTTCCCTCCAAA
GCCTAAGGACACCCTGATGATCTCTCGGACCCCTGAAGTGACCTGCGTGGTGGTG
GATGTGTCTCACGAGGACCCAGAAGTGAAGTTCAATTGGTACGTGGACGGCGTGG
AAGTGCACAACGCCAAGACCAAGCCTAGAGAGGAACAGTACAACTCCACCTACAG
AGTGGTGTCCGTGCTGACCGTGCTGCACCAGGATTGGCTGAACGGCAAAGAGTAC
AAGTGCAAGGTGTCCAACAAGGCCCTGCCTGCTCCTATCGAAAAGACCATCTCCA
AGGCCAAGGGCCAGCCTAGGGAACCCCAGGTTTACACCTTGCCTCCAAGCCGGGA
AGAGATGACCAAGAACCAGGTGTCCCTGACCTGCCTCGTGAAGGGCTTCTACCCT
TCCGATATCGCCGTGGAATGGGAGAGCAATGGCCAGCCTGAGAACAACTACAAGA
CCACTCCTCCTGTGCTGGACTCCGACGGCTCATTCTTCCTGTACTCCAAGCTGAC
AGTGGACAAGTCCAGATGGCAGCAGGGCAACGTGTTCTCCTGCTCCGTGATGCAC
GAGGCCCTGCACAATCACTACACACAGAAGTCCCTGTCTCTGTCCCCTGGCAAGT
AA
SEQ ID NO: 36-712O_L7_2xGS_DNA sequence
ATGGAATTCGGCCTGTCTTGGCTGTTCCTGGTGGCCATTCTGAAGGGCGCTCTGG
CCGCTCAGACCAACTTCATGCCTATGGACGACCTGGAACAGCGGCTGTACGAGCA
GTTCATCCTGCAGCAAGGACTGGAAGGCGGCGGAGGATCTGGAGGCGGTGGATCT
GAAGTGCAGCTGGTTGAAAGTGGCGGCGGATTGGTTCAGCCTGGCGGATCTCTGA
GACTGTCTTGTGCCGCCTCTGGCTACGACTTCACCCACTACGGCATGAATTGGGT
CCGACAGGCTCCCGGCAAAGGCCTGGAATGGGTCGGATGGATCAACACCTATACC
GGCGAGCCTACCTACGCCGCCGATTTCAAGCGGAGATTCACCTTCTCCCTGGACA
CCTCCAAGTCTACCGCCTACCTGCAGATGAACTCCCTGAGAGCCGAGGACACCGC
CGTGTACTACTGCGCTAAGTACCCCTACTACTACGGCACCAGCCACTGGTACTTC
GACGTGTGGGGACAGGGCACACTGGTCACAGTGTCCTCCGCCTCTACCAAGGGAC
CCTCTGTGTTTCCTCTGGCTCCCTCCAGCAAGTCCACCTCTGGTGGAACAGCTGC
TCTGGGCTGCCTGGTCAAGGACTACTTTCCTGAGCCTGTGACCGTGTCCTGGGCT
TCTGGTGCTCTGACATCTGGCGTGCACACCTTTCCAGCTGTGCTGCAGTCCTCCG
GCCTGTACTCTCTGTCCTCTGTCGTGACCGTGCCTTCCAGCTCTCTGGGAACCCA
GACCTACATCTGCAATGTGAACCACAAGCCTTCCAACACCAAGGTCGACAAGAAG
GTGGAACCCAAGTCCTGCGATAAGACCCACACCTGTCCTCCATGTCCTGCTCCAG
AAGCTGCTGGCGGCCCATCCGTGTTTCTGTTCCCTCCAAAGCCTAAGGACACCCT
GATGATCTCTCGGACCCCTGAAGTGACCTGCGTGGTGGTGGATGTGTCTCACGAG
GACCCAGAAGTGAAGTTCAATTGGTACGTGGACGGCGTGGAAGTGCACAACGCCA
AGACCAAGCCTAGAGAGGAACAGTACAACTCCACCTACAGAGTGGTGTCCGTGCT
GACCGTGCTGCACCAGGATTGGCTGAACGGCAAAGAGTACAAGTGCAAGGTGTCC
AACAAGGCCCTGCCTGCTCCTATCGAAAAGACCATCTCCAAGGCCAAGGGCCAGC
CTAGGGAACCCCAGGTTTACACCTTGCCTCCAAGCCGGGAAGAGATGACCAAGAA
CCAGGTGTCCCTGACCTGCCTCGTGAAGGGCTTCTACCCTTCCGATATCGCCGTG
GAATGGGAGAGCAATGGCCAGCCTGAGAACAACTACAAGACCACTCCTCCTGTGC
TGGACTCCGACGGCTCATTCTTCCTGTACTCCAAGCTGACAGTGGACAAGTCCAG
ATGGCAGCAGGGCAACGTGTTCTCCTGCTCCGTGATGCACGAGGCCCTGCACAAT
CACTACACACAGAAGTCCCTGTCTCTGTCCCCTGGCAAGTAA
SEQ ID NO: 37-712O_L7_2xGS_Protein sequence
MEFGLSWLFLVAILKGALAAQTNFMPMDDLEQRLYEQFILQQGLEGGGGSGGGGS
EVQLVESGGGLVQPGGSLRLSCAASGYDFTHYGMNWVRQAPGKGLEWVGWINT
YTGEPTYAADFKRRFTFSLDTSKSTAYLQMNSLRAEDTAVYYCAKYPYYYGTSHV
VYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHE
ALHNHYTQKSLSLSPGK
SEQ ID NO: 38-712O_L7_1xGS_DNA sequence
ATGGAATTCGGCCTGTCTTGGCTGTTCCTGGTGGCCATTCTGAAGGGCGCTCTGG
CCGCTCAGACCAACTTCATGCCTATGGACGACCTGGAACAGCGGCTGTACGAGCA
GTTCATCCTGCAGCAAGGACTGGAAGGAGGCGGTGGATCTGAAGTGCAGCTGGTT
GAAAGTGGCGGCGGATTGGTTCAGCCTGGCGGATCTCTGAGACTGTCTTGTGCCG
CCTCTGGCTACGACTTCACCCACTACGGCATGAATTGGGTCCGACAGGCTCCCGG
CAAAGGCCTGGAATGGGTCGGATGGATCAACACCTATACCGGCGAGCCTACCTAC
GCCGCCGATTTCAAGCGGAGATTCACCTTCTCCCTGGACACCTCCAAGTCTACCG
CCTACCTGCAGATGAACTCCCTGAGAGCCGAGGACACCGCCGTGTACTACTGCGC
TAAGTACCCCTACTACTACGGCACCAGCCACTGGTACTTCGACGTGTGGGGACAG
GGCACACTGGTCACAGTGTCCTCCGCCTCTACCAAGGGACCCTCTGTGTTTCCTC
TGGCTCCCTCCAGCAAGTCCACCTCTGGTGGAACAGCTGCTCTGGGCTGCCTGGT
CAAGGACTACTTTCCTGAGCCTGTGACCGTGTCCTGGGCTTCTGGTGCTCTGACA
TCTGGCGTGCACACCTTTCCAGCTGTGCTGCAGTCCTCCGGCCTGTACTCTCTGT
CCTCTGTCGTGACCGTGCCTTCCAGCTCTCTGGGAACCCAGACCTACATCTGCAA
TGTGAACCACAAGCCTTCCAACACCAAGGTCGACAAGAAGGTGGAACCCAAGTCC
TGCGATAAGACCCACACCTGTCCTCCATGTCCTGCTCCAGAAGCTGCTGGCGGCC
CATCCGTGTTTCTGTTCCCTCCAAAGCCTAAGGACACCCTGATGATCTCTCGGAC
CCCTGAAGTGACCTGCGTGGTGGTGGATGTGTCTCACGAGGACCCAGAAGTGAAG
TTCAATTGGTACGTGGACGGCGTGGAAGTGCACAACGCCAAGACCAAGCCTAGAG
AGGAACAGTACAACTCCACCTACAGAGTGGTGTCCGTGCTGACCGTGCTGCACCA
GGATTGGCTGAACGGCAAAGAGTACAAGTGCAAGGTGTCCAACAAGGCCCTGCCT
GCTCCTATCGAAAAGACCATCTCCAAGGCCAAGGGCCAGCCTAGGGAACCCCAGG
TTTACACCTTGCCTCCAAGCCGGGAAGAGATGACCAAGAACCAGGTGTCCCTGAC
CTGCCTCGTGAAGGGCTTCTACCCTTCCGATATCGCCGTGGAATGGGAGAGCAAT
GGCCAGCCTGAGAACAACTACAAGACCACTCCTCCTGTGCTGGACTCCGACGGCT
CATTCTTCCTGTACTCCAAGCTGACAGTGGACAAGTCCAGATGGCAGCAGGGCAA
CGTGTTCTCCTGCTCCGTGATGCACGAGGCCCTGCACAATCACTACACACAGAAG
TCCCTGTCTCTGTCCCCTGGCAAGTAA
SEQ ID NO: 39-712O_L7_1xGS_DNA sequence
MEFGLSWLFLVAILKGALAAQTNFMPMDDLEQRLYEQFILQQGLEGGGGSEVQLV
ESGGGLVQPGGSLRLSCAASGYDFTHYGMNWVRQAPGKGLEWVGWINTYTGEP
TYAADFKRRFTFSLDTSKSTAYLQMNSLRAEDTAVYYCAKYPYYYGTSHWYFDV
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNH
YTQKSLSLSPGK
SEQ ID NO: 40 712O_L10_3xGS_DNA sequence
ATGGAATTCGGCCTGTCTTGGCTGTTCCTGGTGGCCATTCTGAAGGGCGCTTTGG
CCGCTCAGCAGAAGTTTCAGCCTCTGGACGAGCTGGAACAGACCCTGTACGAGCA
GTTCATGCTCCAGCAGGCTTTGGAAGGCGGCGGAGGATCTGGCGGAGGCGGTAGC
GGAGGCGGTGGATCTGAAGTGCAGCTGGTTGAAAGTGGCGGCGGATTGGTTCAGC
CTGGCGGATCTCTGAGACTGTCTTGTGCCGCCTCTGGCTACGACTTCACCCACTA
CGGCATGAATTGGGTCCGACAGGCTCCCGGCAAAGGCCTGGAATGGGTCGGATGG
ATCAACACCTATACCGGCGAGCCTACCTACGCCGCCGATTTCAAGCGGAGATTCA
CCTTCTCCCTGGACACCTCCAAGTCTACCGCCTACCTGCAGATGAACTCCCTGAG
AGCCGAGGACACCGCCGTGTACTACTGCGCTAAGTACCCCTACTACTACGGCACC
AGCCACTGGTACTTCGACGTGTGGGGACAGGGCACACTGGTCACAGTGTCCTCCG
CCTCTACCAAGGGACCCTCTGTGTTTCCTCTGGCTCCCTCCAGCAAGTCCACCTC
TGGTGGAACAGCTGCTCTGGGCTGCCTGGTCAAGGACTACTTTCCTGAGCCTGTG
ACCGTGTCCTGGGCTTCTGGTGCTCTGACATCTGGCGTGCACACCTTTCCAGCTG
TGCTGCAGTCCTCCGGCCTGTACTCTCTGTCCTCTGTCGTGACCGTGCCTTCCAG
CTCTCTGGGAACCCAGACCTACATCTGCAATGTGAACCACAAGCCTTCCAACACC
AAGGTCGACAAGAAGGTGGAACCCAAGTCCTGCGATAAGACCCACACCTGTCCTC
CATGTCCTGCTCCAGAAGCTGCTGGCGGCCCATCCGTGTTTCTGTTCCCTCCAAA
GCCTAAGGACACCCTGATGATCTCTCGGACCCCTGAAGTGACCTGCGTGGTGGTG
GATGTGTCTCACGAGGACCCAGAAGTGAAGTTCAATTGGTACGTGGACGGCGTGG
AAGTGCACAACGCCAAGACCAAGCCTAGAGAGGAACAGTACAACTCCACCTACAG
AGTGGTGTCCGTGCTGACCGTGCTGCACCAGGATTGGCTGAACGGCAAAGAGTAC
AAGTGCAAGGTGTCCAACAAGGCCCTGCCTGCTCCTATCGAAAAGACCATCTCCA
AGGCCAAGGGCCAGCCTAGGGAACCCCAGGTTTACACCTTGCCTCCAAGCCGGGA
AGAGATGACCAAGAACCAGGTGTCCCTGACCTGCCTCGTGAAGGGCTTCTACCCT
TCCGATATCGCCGTGGAATGGGAGAGCAATGGCCAGCCTGAGAACAACTACAAGA
CCACTCCTCCTGTGCTGGACTCCGACGGCTCATTCTTCCTGTACTCCAAGCTGAC
AGTGGACAAGTCCAGATGGCAGCAGGGCAACGTGTTCTCCTGCTCCGTGATGCAC
GAGGCCCTGCACAATCACTACACACAGAAGTCCCTGTCTCTGTCCCCTGGCAAGT
AA
SEQ ID NO: 41 712O_L10_3xGS_Protein sequence
MEFGLSWLFLVAILKGALAAQQKFQPLDELEQTLYEQFMLQQALEGGGGSGGGGS
GGGGSEVQLVESGGGLVQPGGSLRLSCAASGYDFTHYGMNWVRQAPGKGLEWV
GWINTYTGEPTYAADFKRRFTFSLDTSKSTAYLQMNSLRAEDTAVYYCAKYPYYY
GTSHWYFDVWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKP
SNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSC
SVMHEALHNHYTQKSLSLSPGK
SEQ ID NO: 42 712O_L10_2xGS_DNA sequence
ATGGAATTCGGCCTGTCTTGGCTGTTCCTGGTGGCCATTCTGAAGGGCGCTTTGG
CCGCTCAGCAGAAGTTTCAGCCTCTGGACGAGCTGGAACAGACCCTGTACGAGCA
GTTCATGCTCCAGCAGGCTTTGGAAGGCGGCGGAGGATCTGGAGGCGGTGGATCT
GAAGTGCAGCTGGTTGAAAGTGGCGGCGGATTGGTTCAGCCTGGCGGATCTCTGA
GACTGTCTTGTGCCGCCTCTGGCTACGACTTCACCCACTACGGCATGAATTGGGT
CCGACAGGCTCCCGGCAAAGGCCTGGAATGGGTCGGATGGATCAACACCTATACC
GGCGAGCCTACCTACGCCGCCGATTTCAAGCGGAGATTCACCTTCTCCCTGGACA
CCTCCAAGTCTACCGCCTACCTGCAGATGAACTCCCTGAGAGCCGAGGACACCGC
CGTGTACTACTGCGCTAAGTACCCCTACTACTACGGCACCAGCCACTGGTACTTC
GACGTGTGGGGACAGGGCACACTGGTCACAGTGTCCTCCGCCTCTACCAAGGGAC
CCTCTGTGTTTCCTCTGGCTCCCTCCAGCAAGTCCACCTCTGGTGGAACAGCTGC
TCTGGGCTGCCTGGTCAAGGACTACTTTCCTGAGCCTGTGACCGTGTCCTGGGCT
TCTGGTGCTCTGACATCTGGCGTGCACACCTTTCCAGCTGTGCTGCAGTCCTCCG
GCCTGTACTCTCTGTCCTCTGTCGTGACCGTGCCTTCCAGCTCTCTGGGAACCCA
GACCTACATCTGCAATGTGAACCACAAGCCTTCCAACACCAAGGTCGACAAGAAG
GTGGAACCCAAGTCCTGCGATAAGACCCACACCTGTCCTCCATGTCCTGCTCCAG
AAGCTGCTGGCGGCCCATCCGTGTTTCTGTTCCCTCCAAAGCCTAAGGACACCCT
GATGATCTCTCGGACCCCTGAAGTGACCTGCGTGGTGGTGGATGTGTCTCACGAG
GACCCAGAAGTGAAGTTCAATTGGTACGTGGACGGCGTGGAAGTGCACAACGCCA
AGACCAAGCCTAGAGAGGAACAGTACAACTCCACCTACAGAGTGGTGTCCGTGCT
GACCGTGCTGCACCAGGATTGGCTGAACGGCAAAGAGTACAAGTGCAAGGTGTCC
AACAAGGCCCTGCCTGCTCCTATCGAAAAGACCATCTCCAAGGCCAAGGGCCAGC
CTAGGGAACCCCAGGTTTACACCTTGCCTCCAAGCCGGGAAGAGATGACCAAGAA
CCAGGTGTCCCTGACCTGCCTCGTGAAGGGCTTCTACCCTTCCGATATCGCCGTG
GAATGGGAGAGCAATGGCCAGCCTGAGAACAACTACAAGACCACTCCTCCTGTGC
TGGACTCCGACGGCTCATTCTTCCTGTACTCCAAGCTGACAGTGGACAAGTCCAG
ATGGCAGCAGGGCAACGTGTTCTCCTGCTCCGTGATGCACGAGGCCCTGCACAAT
CACTACACACAGAAGTCCCTGTCTCTGTCCCCTGGCAAGTAA
SEQ ID NO: 43 712O_L10_2xGS_Protein sequence
MEFGLSWLFLVAILKGALAAQQKFQPLDELEQTLYEQFMLQQALEGGGGSGGGGS
EVQLVESGGGLVQPGGSLRLSCAASGYDFTHYGMNWVRQAPGKGLEWVGWINT
YTGEPTYAADFKRRFTFSLDTSKSTAYLQMNSLRAEDTAVYYCAKYPYYYGTSHV
VYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHE
ALHNHYTQKSLSLSPGK
SEQ ID NO: 44 712O_L10_3xGS_DNA sequence
ATGGAATTCGGCCTGTCTTGGCTGTTCCTGGTGGCCATTCTGAAGGGCGCTTTGG
CCGCTCAGCAGAAGTTTCAGCCTCTGGACGAGCTGGAACAGACCCTGTACGAGCA
GTTCATGCTCCAGCAGGCTTTGGAAGGAGGCGGTGGATCTGAAGTGCAGCTGGTT
GAAAGTGGCGGCGGATTGGTTCAGCCTGGCGGATCTCTGAGACTGTCTTGTGCCG
CCTCTGGCTACGACTTCACCCACTACGGCATGAATTGGGTCCGACAGGCTCCCGG
CAAAGGCCTGGAATGGGTCGGATGGATCAACACCTATACCGGCGAGCCTACCTAC
GCCGCCGATTTCAAGCGGAGATTCACCTTCTCCCTGGACACCTCCAAGTCTACCG
CCTACCTGCAGATGAACTCCCTGAGAGCCGAGGACACCGCCGTGTACTACTGCGC
TAAGTACCCCTACTACTACGGCACCAGCCACTGGTACTTCGACGTGTGGGGACAG
GGCACACTGGTCACAGTGTCCTCCGCCTCTACCAAGGGACCCTCTGTGTTTCCTC
TGGCTCCCTCCAGCAAGTCCACCTCTGGTGGAACAGCTGCTCTGGGCTGCCTGGT
CAAGGACTACTTTCCTGAGCCTGTGACCGTGTCCTGGGCTTCTGGTGCTCTGACA
TCTGGCGTGCACACCTTTCCAGCTGTGCTGCAGTCCTCCGGCCTGTACTCTCTGT
CCTCTGTCGTGACCGTGCCTTCCAGCTCTCTGGGAACCCAGACCTACATCTGCAA
TGTGAACCACAAGCCTTCCAACACCAAGGTCGACAAGAAGGTGGAACCCAAGTCC
TGCGATAAGACCCACACCTGTCCTCCATGTCCTGCTCCAGAAGCTGCTGGCGGCC
CATCCGTGTTTCTGTTCCCTCCAAAGCCTAAGGACACCCTGATGATCTCTCGGAC
CCCTGAAGTGACCTGCGTGGTGGTGGATGTGTCTCACGAGGACCCAGAAGTGAAG
TTCAATTGGTACGTGGACGGCGTGGAAGTGCACAACGCCAAGACCAAGCCTAGAG
AGGAACAGTACAACTCCACCTACAGAGTGGTGTCCGTGCTGACCGTGCTGCACCA
GGATTGGCTGAACGGCAAAGAGTACAAGTGCAAGGTGTCCAACAAGGCCCTGCCT
GCTCCTATCGAAAAGACCATCTCCAAGGCCAAGGGCCAGCCTAGGGAACCCCAGG
TTTACACCTTGCCTCCAAGCCGGGAAGAGATGACCAAGAACCAGGTGTCCCTGAC
CTGCCTCGTGAAGGGCTTCTACCCTTCCGATATCGCCGTGGAATGGGAGAGCAAT
GGCCAGCCTGAGAACAACTACAAGACCACTCCTCCTGTGCTGGACTCCGACGGCT
CATTCTTCCTGTACTCCAAGCTGACAGTGGACAAGTCCAGATGGCAGCAGGGCAA
CGTGTTCTCCTGCTCCGTGATGCACGAGGCCCTGCACAATCACTACACACAGAAG
TCCCTGTCTCTGTCCCCTGGCAAGTAA
SEQ ID NO: 45 712O_L10_1xGS_Protein sequence
MEFGLSWLFLVAILKGALAAQQKFQPLDELEQTLYEQFMLQQALEGGGGSEVQLV
ESGGGLVQPGGSLRLSCAASGYDFTHYGMNWVRQAPGKGLEWVGWINTYTGEP
TYAADFKRRFTFSLDTSKSTAYLQMNSLRAEDTAVYYCAKYPYYYGTSHWYFDV
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNH
YTQKSLSLSPGK
SEQ ID NO: 46 712O_C terminal L7_1xGS_DNA sequence
ATGGAATTCGGCCTGTCTTGGCTGTTCCTGGTGGCCATTCTGAAGGGCGCTCTGG
CCGAAGTGCAGTTGGTTGAATCTGGTGGCGGATTGGTGCAGCCTGGCGGATCTCT
GAGACTGTCTTGTGCCGCCTCTGGCTACGATTTCACCCACTACGGCATGAATTGG
GTCCGACAGGCTCCTGGCAAAGGCCTGGAATGGGTCGGATGGATCAATACCTATA
CCGGCGAGCCTACCTACGCCGCCGACTTCAAGAGAAGATTCACCTTCTCCCTGGA
CACCTCCAAGTCTACCGCCTACCTGCAGATGAACTCCCTGAGAGCTGAGGACACC
GCCGTGTACTACTGCGCTAAGTACCCCTACTACTACGGCACCAGCCACTGGTACT
TTGATGTGTGGGGACAGGGCACCCTGGTCACCGTTTCTTCCGCTTCTACAAAGGG
ACCCAGCGTGTTCCCTCTGGCTCCTAGCTCTAAGTCTACCTCTGGCGGAACCGCT
GCTCTGGGCTGTCTGGTCAAGGATTACTTCCCTGAGCCTGTGACCGTGTCCTGGA
ATAGTGGTGCTCTGACATCCGGCGTGCACACCTTTCCAGCTGTGCTGCAGTCCTC
TGGCCTGTACTCTCTGTCCTCTGTCGTGACCGTCCCTTCTAGCTCTCTGGGCACC
CAGACCTACATCTGCAACGTGAACCACAAGCCTTCCAACACTAAGGTGGACAAGA
AGGTGGAACCCAAGTCCTGCGATAAGACCCACACCTGTCCTCCATGTCCTGCACC
TGAAGCTGCTGGCGGACCCTCTGTGTTCCTGTTTCCTCCAAAGCCTAAGGACACC
CTGATGATCTCTCGGACCCCTGAAGTGACCTGCGTGGTGGTGGATGTGTCTCACG
AGGACCCAGAAGTGAAGTTCAATTGGTACGTGGACGGCGTGGAAGTGCACAACGC
CAAGACCAAGCCTAGAGAGGAACAGTACAACTCCACCTACAGAGTGGTGTCCGTG
CTGACCGTGCTGCACCAGGATTGGCTGAACGGCAAAGAGTACAAGTGCAAGGTGT
CCAACAAGGCCCTGCCTGCTCCTATCGAAAAGACCATCTCTAAGGCTAAGGGCCA
GCCTCGGGAACCTCAGGTTTACACACTGCCTCCAAGCCGGGAAGAGATGACCAAG
AATCAGGTGTCCCTGACCTGCCTCGTGAAGGGCTTCTACCCTTCCGATATCGCCG
TCGAATGGGAGTCCAATGGCCAGCCTGAGAACAACTACAAGACAACCCCTCCTGT
GCTGGACTCCGACGGCTCATTCTTCCTGTACTCCAAGCTGACAGTGGACAAGTCT
CGGTGGCAGCAGGGCAACGTGTTCTCCTGTTCTGTGATGCACGAGGCCCTGCACA
ACCACTACACACAGAAGTCACTCTCCCTTTCCCCGGGCgctGGCGGCGGAGGATC
TGCTCAGACCAACTTCATGCCTATGGACGACCTGGAACAGCGGCTGTACGAGCAG
TTCATCCTGCAGCAAGGACTGGAAtga
SEQ ID NO: 47 712O_C terminal L7_1xGS_Protein sequence
MEFGLSWLFLVAILKGALAEVQLVESGGGLVQPGGSLRLSCAASGYDFTHYGMNW
VRQAPGKGLEWVGWINTYTGEPTYAADFKRRFTFSLDTSKSTAYLQMNSLRAE
DTAVYYCAKYPYYYGTSHWYFDVWGQGTLVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLT
VDKSRWQQGNVFSCSVMHEALHNHYTQKSLSLSPGAGGGGSAQTNFMPMDDLEQR
LYEQFILQQGLE*
SEQ ID NO: 48 712O_L7_C_2xGS_DNA sequence
ATGGAATTCGGCCTGTCTTGGCTGTTCCTGGTGGCCATTCTGAAGGGCGCTCTGG
CCGAAGTGCAGTTGGTTGAATCTGGTGGCGGATTGGTGCAGCCTGGCGGATCTCT
GAGACTGTCTTGTGCCGCCTCTGGCTACGATTTCACCCACTACGGCATGAATTGG
GTCCGACAGGCTCCTGGCAAAGGCCTGGAATGGGTCGGATGGATCAATACCTATA
CCGGCGAGCCTACCTACGCCGCCGACTTCAAGAGAAGATTCACCTTCTCCCTGGA
CACCTCCAAGTCTACCGCCTACCTGCAGATGAACTCCCTGAGAGCTGAGGACACC
GCCGTGTACTACTGCGCTAAGTACCCCTACTACTACGGCACCAGCCACTGGTACT
TTGATGTGTGGGGACAGGGCACCCTGGTCACCGTTTCTTCCGCTTCTACAAAGGG
ACCCAGCGTGTTCCCTCTGGCTCCTAGCTCTAAGTCTACCTCTGGCGGAACCGCT
GCTCTGGGCTGTCTGGTCAAGGATTACTTCCCTGAGCCTGTGACCGTGTCCTGGA
ATAGTGGTGCTCTGACATCCGGCGTGCACACCTTTCCAGCTGTGCTGCAGTCCTC
TGGCCTGTACTCTCTGTCCTCTGTCGTGACCGTCCCTTCTAGCTCTCTGGGCACC
CAGACCTACATCTGCAACGTGAACCACAAGCCTTCCAACACTAAGGTGGACAAGA
AGGTGGAACCCAAGTCCTGCGATAAGACCCACACCTGTCCTCCATGTCCTGCACC
TGAAGCTGCTGGCGGACCCTCTGTGTTCCTGTTTCCTCCAAAGCCTAAGGACACC
CTGATGATCTCTCGGACCCCTGAAGTGACCTGCGTGGTGGTGGATGTGTCTCACG
AGGACCCAGAAGTGAAGTTCAATTGGTACGTGGACGGCGTGGAAGTGCACAACGC
CAAGACCAAGCCTAGAGAGGAACAGTACAACTCCACCTACAGAGTGGTGTCCGTG
CTGACCGTGCTGCACCAGGATTGGCTGAACGGCAAAGAGTACAAGTGCAAGGTGT
CCAACAAGGCCCTGCCTGCTCCTATCGAAAAGACCATCTCTAAGGCTAAGGGCCA
GCCTCGGGAACCTCAGGTTTACACACTGCCTCCAAGCCGGGAAGAGATGACCAAG
AATCAGGTGTCCCTGACCTGCCTCGTGAAGGGCTTCTACCCTTCCGATATCGCCG
TCGAATGGGAGTCCAATGGCCAGCCTGAGAACAACTACAAGACAACCCCTCCTGT
GCTGGACTCCGACGGCTCATTCTTCCTGTACTCCAAGCTGACAGTGGACAAGTCT
CGGTGGCAGCAGGGCAACGTGTTCTCCTGTTCTGTGATGCACGAGGCCCTGCACA
ACCACTACACACAGAAGTCACTCTCCCTTTCCCCGGGCgctGGCGGCGGAGGATC
TGGCGGAGGCGGTAGCGCTCAGACCAACTTCATGCCTATGGACGACCTGGAACAG
CGGCTGTACGAGCAGTTCATCCTGCAGCAAGGACTGGAAtga
SEQ ID NO: 49 712O_L7_C_2xGS_protein sequence
MEFGLSWLFLVAILKGALAEVQLVESGGGLVQPGGSLRLSCAASGYDFTHYGMNW
VRQAPGKGLEWVGWINTYTGEPTYAADFKRRFTFSLDTSKSTAYLQMNSLRAE
DTAVYYCAKYPYYYGTSHWYFDVWGQGTLVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLT
VDKSRWQQGNVFSCSVMHEALHNHYTQKSLSLSPGAGGGGSGGGGSAQTNFMPMD
DLEQRLYEQFILQQGLE
SEQ ID NO: 50 712O_L7_C_3xGS_DNA sequence
ATGGAATTCGGCCTGTCTTGGCTGTTCCTGGTGGCCATTCTGAAGGGCGCTCTGG
CCGAAGTGCAGTTGGTTGAATCTGGTGGCGGATTGGTGCAGCCTGGCGGATCTCT
GAGACTGTCTTGTGCCGCCTCTGGCTACGATTTCACCCACTACGGCATGAATTGG
GTCCGACAGGCTCCTGGCAAAGGCCTGGAATGGGTCGGATGGATCAATACCTATA
CCGGCGAGCCTACCTACGCCGCCGACTTCAAGAGAAGATTCACCTTCTCCCTGGA
CACCTCCAAGTCTACCGCCTACCTGCAGATGAACTCCCTGAGAGCTGAGGACACC
GCCGTGTACTACTGCGCTAAGTACCCCTACTACTACGGCACCAGCCACTGGTACT
TTGATGTGTGGGGACAGGGCACCCTGGTCACCGTTTCTTCCGCTTCTACAAAGGG
ACCCAGCGTGTTCCCTCTGGCTCCTAGCTCTAAGTCTACCTCTGGCGGAACCGCT
GCTCTGGGCTGTCTGGTCAAGGATTACTTCCCTGAGCCTGTGACCGTGTCCTGGA
ATAGTGGTGCTCTGACATCCGGCGTGCACACCTTTCCAGCTGTGCTGCAGTCCTC
TGGCCTGTACTCTCTGTCCTCTGTCGTGACCGTCCCTTCTAGCTCTCTGGGCACC
CAGACCTACATCTGCAACGTGAACCACAAGCCTTCCAACACTAAGGTGGACAAGA
AGGTGGAACCCAAGTCCTGCGATAAGACCCACACCTGTCCTCCATGTCCTGCACC
TGAAGCTGCTGGCGGACCCTCTGTGTTCCTGTTTCCTCCAAAGCCTAAGGACACC
CTGATGATCTCTCGGACCCCTGAAGTGACCTGCGTGGTGGTGGATGTGTCTCACG
AGGACCCAGAAGTGAAGTTCAATTGGTACGTGGACGGCGTGGAAGTGCACAACGC
CAAGACCAAGCCTAGAGAGGAACAGTACAACTCCACCTACAGAGTGGTGTCCGTG
CTGACCGTGCTGCACCAGGATTGGCTGAACGGCAAAGAGTACAAGTGCAAGGTGT
CCAACAAGGCCCTGCCTGCTCCTATCGAAAAGACCATCTCTAAGGCTAAGGGCCA
GCCTCGGGAACCTCAGGTTTACACACTGCCTCCAAGCCGGGAAGAGATGACCAAG
AATCAGGTGTCCCTGACCTGCCTCGTGAAGGGCTTCTACCCTTCCGATATCGCCG
TCGAATGGGAGTCCAATGGCCAGCCTGAGAACAACTACAAGACAACCCCTCCTGT
GCTGGACTCCGACGGCTCATTCTTCCTGTACTCCAAGCTGACAGTGGACAAGTCT
CGGTGGCAGCAGGGCAACGTGTTCTCCTGTTCTGTGATGCACGAGGCCCTGCACA
ACCACTACACACAGAAGTCACTCTCCCTTTCCCCGGGCgctGGCGGCGGAGGATC
TGGCGGAGGCGGTAGCGGTGGTGGTGGATCTGCTCAGACCAACTTCATGCCTATG
GACGACCTGGAACAGCGGCTGTACGAGCAGTTCATCCTGCAGCAAGGACTGGAAt
ga
> SEQ ID NO: 51 712O_L7_C_3xGS_Protein sequence
MEFGLSWLFLVAILKGALAEVQLVESGGGLVQPGGSLRLSCAASGYDFTHYGMN
WVRQAPGKGLEWVGWINTYTGEPTYAADFKRRFTFSLDTSKSTAYLQMNSLRAE
DTAVYYCAKYPYYYGTSHWYFDVWGQGTLVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLT
VDKSRWQQGNVFSCSVMHEALHNHYTQKSLSLSPGAGGGGSGGGGSGGGGSAQTN
FMPMDDLEQRLYEQFILQQGLE
SEQ ID NO: 52 712O_L15_C_1xGS_DNA sequence
ATGGAATTCGGCCTGTCTTGGCTGTTCCTGGTGGCCATTCTGAAGGGCGCTCTGG
CCGAAGTGCAGTTGGTTGAATCTGGTGGCGGATTGGTGCAGCCTGGCGGATCTCT
GAGACTGTCTTGTGCCGCCTCTGGCTACGATTTCACCCACTACGGCATGAATTGG
GTCCGACAGGCTCCTGGCAAAGGCCTGGAATGGGTCGGATGGATCAATACCTATA
CCGGCGAGCCTACCTACGCCGCCGACTTCAAGAGAAGATTCACCTTCTCCCTGGA
CACCTCCAAGTCTACCGCCTACCTGCAGATGAACTCCCTGAGAGCTGAGGACACC
GCCGTGTACTACTGCGCTAAGTACCCCTACTACTACGGCACCAGCCACTGGTACT
TTGATGTGTGGGGACAGGGCACCCTGGTCACCGTTTCTTCCGCTTCTACAAAGGG
ACCCAGCGTGTTCCCTCTGGCTCCTAGCTCTAAGTCTACCTCTGGCGGAACCGCT
GCTCTGGGCTGTCTGGTCAAGGATTACTTCCCTGAGCCTGTGACCGTGTCCTGGA
ATAGTGGTGCTCTGACATCCGGCGTGCACACCTTTCCAGCTGTGCTGCAGTCCTC
TGGCCTGTACTCTCTGTCCTCTGTCGTGACCGTCCCTTCTAGCTCTCTGGGCACC
CAGACCTACATCTGCAACGTGAACCACAAGCCTTCCAACACTAAGGTGGACAAGA
AGGTGGAACCCAAGTCCTGCGATAAGACCCACACCTGTCCTCCATGTCCTGCACC
TGAAGCTGCTGGCGGACCCTCTGTGTTCCTGTTTCCTCCAAAGCCTAAGGACACC
CTGATGATCTCTCGGACCCCTGAAGTGACCTGCGTGGTGGTGGATGTGTCTCACG
AGGACCCAGAAGTGAAGTTCAATTGGTACGTGGACGGCGTGGAAGTGCACAACGC
CAAGACCAAGCCTAGAGAGGAACAGTACAACTCCACCTACAGAGTGGTGTCCGTG
CTGACCGTGCTGCACCAGGATTGGCTGAACGGCAAAGAGTACAAGTGCAAGGTGT
CCAACAAGGCCCTGCCTGCTCCTATCGAAAAGACCATCTCTAAGGCTAAGGGCCA
GCCTCGGGAACCTCAGGTTTACACACTGCCTCCAAGCCGGGAAGAGATGACCAAG
AATCAGGTGTCCCTGACCTGCCTCGTGAAGGGCTTCTACCCTTCCGATATCGCCG
TCGAATGGGAGTCCAATGGCCAGCCTGAGAACAACTACAAGACAACCCCTCCTGT
GCTGGACTCCGACGGCTCATTCTTCCTGTACTCCAAGCTGACAGTGGACAAGTCT
CGGTGGCAGCAGGGCAACGTGTTCTCCTGTTCTGTGATGCACGAGGCCCTGCACA
ACCACTACACACAGAAGTCACTCTCCCTTTCCCCGGGCgctGGCGGCGGAGGATC
TGCCCAGCAGAAGTATCAGCCTCTGGACGAGCTGGACAAGACCCTGTACGACCAG
TTCATGCTCCAGCAGGGACTGGAAtga
SEQ ID NO: 53 712O_L15_C_1xGS_Protein sequence
MEFGLSWLFLVAILKGALAEVQLVESGGGLVQPGGSLRLSCAASGYDFTHYGMN
WVRQAPGKGLEWVGWINTYTGEPTYAADFKRRFTFSLDTSKSTAYLQMNSLRAE
DTAVYYCAKYPYYYGTSHWYFDVWGQGTLVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLT
VDKSRWQQGNVFSCSVMHEALHNHYTQKSLSLSPGAGGGGSAQQKYQPLDELDKT
LYDQFMLQQGLE
SEQ ID NO: 54 712O_L15_C_2xGS_DNA sequence
TGGAATTCGGCCTGTCTTGGCTGTTCCTGGTGGCCATTCTGAAGGGCGCTCTGGC
CGAAGTGCAGTTGGTTGAATCTGGTGGCGGATTGGTGCAGCCTGGCGGATCTCTG
AGACTGTCTTGTGCCGCCTCTGGCTACGATTTCACCCACTACGGCATGAATTGGG
TCCGACAGGCTCCTGGCAAAGGCCTGGAATGGGTCGGATGGATCAATACCTATAC
CGGCGAGCCTACCTACGCCGCCGACTTCAAGAGAAGATTCACCTTCTCCCTGGAC
ACCTCCAAGTCTACCGCCTACCTGCAGATGAACTCCCTGAGAGCTGAGGACACCG
CCGTGTACTACTGCGCTAAGTACCCCTACTACTACGGCACCAGCCACTGGTACTT
TGATGTGTGGGGACAGGGCACCCTGGTCACCGTTTCTTCCGCTTCTACAAAGGGA
CCCAGCGTGTTCCCTCTGGCTCCTAGCTCTAAGTCTACCTCTGGCGGAACCGCTG
CTCTGGGCTGTCTGGTCAAGGATTACTTCCCTGAGCCTGTGACCGTGTCCTGGAA
TAGTGGTGCTCTGACATCCGGCGTGCACACCTTTCCAGCTGTGCTGCAGTCCTCT
GGCCTGTACTCTCTGTCCTCTGTCGTGACCGTCCCTTCTAGCTCTCTGGGCACCC
AGACCTACATCTGCAACGTGAACCACAAGCCTTCCAACACTAAGGTGGACAAGAA
GGTGGAACCCAAGTCCTGCGATAAGACCCACACCTGTCCTCCATGTCCTGCACCT
GAAGCTGCTGGCGGACCCTCTGTGTTCCTGTTTCCTCCAAAGCCTAAGGACACCC
TGATGATCTCTCGGACCCCTGAAGTGACCTGCGTGGTGGTGGATGTGTCTCACGA
GGACCCAGAAGTGAAGTTCAATTGGTACGTGGACGGCGTGGAAGTGCACAACGCC
AAGACCAAGCCTAGAGAGGAACAGTACAACTCCACCTACAGAGTGGTGTCCGTGC
TGACCGTGCTGCACCAGGATTGGCTGAACGGCAAAGAGTACAAGTGCAAGGTGTC
CAACAAGGCCCTGCCTGCTCCTATCGAAAAGACCATCTCTAAGGCTAAGGGCCAG
CCTCGGGAACCTCAGGTTTACACACTGCCTCCAAGCCGGGAAGAGATGACCAAGA
ATCAGGTGTCCCTGACCTGCCTCGTGAAGGGCTTCTACCCTTCCGATATCGCCGT
CGAATGGGAGTCCAATGGCCAGCCTGAGAACAACTACAAGACAACCCCTCCTGTG
CTGGACTCCGACGGCTCATTCTTCCTGTACTCCAAGCTGACAGTGGACAAGTCTC
GGTGGCAGCAGGGCAACGTGTTCTCCTGTTCTGTGATGCACGAGGCCCTGCACAA
CCACTACACACAGAAGTCACTCTCCCTTTCCCCGGGCgctGGCGGCGGAGGATCT
GGCGGAGGCGGTAGCGCCCAGCAGAAGTATCAGCCTCTGGACGAGCTGGACAAGA
CCCTGTACGACCAGTTCATGCTCCAGCAGGGACTGGAAtga
SEQ ID NO: 55 712O_L15_C_2xGS_Protein sequence
MEFGLSWLFLVAILKGALAEVQLVESGGGLVQPGGSLRLSCAASGYDFTHYGMN
WVRQAPGKGLEWVGWINTYTGEPTYAADFKRRFTFSLDTSKSTAYLQMNSLRAE
DTAVYYCAKYPYYYGTSHWYFDVWGQGTLVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLT
VDKSRWQQGNVFSCSVMHEALHNHYTQKSLSLSPGAGGGGSGGGGSAQQKYQPLD
ELDKTLYDQFMLQQGLE
SEQ ID NO: 56 712O_L15_C_3xGS_DNA sequence
ATGGAATTCGGCCTGTCTTGGCTGTTCCTGGTGGCCATTCTGAAGGGCGCTCTGG
CCGAAGTGCAGTTGGTTGAATCTGGTGGCGGATTGGTGCAGCCTGGCGGATCTCT
GAGACTGTCTTGTGCCGCCTCTGGCTACGATTTCACCCACTACGGCATGAATTGG
GTCCGACAGGCTCCTGGCAAAGGCCTGGAATGGGTCGGATGGATCAATACCTATA
CCGGCGAGCCTACCTACGCCGCCGACTTCAAGAGAAGATTCACCTTCTCCCTGGA
CACCTCCAAGTCTACCGCCTACCTGCAGATGAACTCCCTGAGAGCTGAGGACACC
GCCGTGTACTACTGCGCTAAGTACCCCTACTACTACGGCACCAGCCACTGGTACT
TTGATGTGTGGGGACAGGGCACCCTGGTCACCGTTTCTTCCGCTTCTACAAAGGG
ACCCAGCGTGTTCCCTCTGGCTCCTAGCTCTAAGTCTACCTCTGGCGGAACCGCT
GCTCTGGGCTGTCTGGTCAAGGATTACTTCCCTGAGCCTGTGACCGTGTCCTGGA
ATAGTGGTGCTCTGACATCCGGCGTGCACACCTTTCCAGCTGTGCTGCAGTCCTC
TGGCCTGTACTCTCTGTCCTCTGTCGTGACCGTCCCTTCTAGCTCTCTGGGCACC
CAGACCTACATCTGCAACGTGAACCACAAGCCTTCCAACACTAAGGTGGACAAGA
AGGTGGAACCCAAGTCCTGCGATAAGACCCACACCTGTCCTCCATGTCCTGCACC
TGAAGCTGCTGGCGGACCCTCTGTGTTCCTGTTTCCTCCAAAGCCTAAGGACACC
CTGATGATCTCTCGGACCCCTGAAGTGACCTGCGTGGTGGTGGATGTGTCTCACG
AGGACCCAGAAGTGAAGTTCAATTGGTACGTGGACGGCGTGGAAGTGCACAACGC
CAAGACCAAGCCTAGAGAGGAACAGTACAACTCCACCTACAGAGTGGTGTCCGTG
CTGACCGTGCTGCACCAGGATTGGCTGAACGGCAAAGAGTACAAGTGCAAGGTGT
CCAACAAGGCCCTGCCTGCTCCTATCGAAAAGACCATCTCTAAGGCTAAGGGCCA
GCCTCGGGAACCTCAGGTTTACACACTGCCTCCAAGCCGGGAAGAGATGACCAAG
AATCAGGTGTCCCTGACCTGCCTCGTGAAGGGCTTCTACCCTTCCGATATCGCCG
TCGAATGGGAGTCCAATGGCCAGCCTGAGAACAACTACAAGACAACCCCTCCTGT
GCTGGACTCCGACGGCTCATTCTTCCTGTACTCCAAGCTGACAGTGGACAAGTCT
CGGTGGCAGCAGGGCAACGTGTTCTCCTGTTCTGTGATGCACGAGGCCCTGCACA
ACCACTACACACAGAAGTCACTCTCCCTTTCCCCGGGCgctGGCGGCGGAGGATC
TGGCGGAGGCGGTAGCGGTGGTGGTGGATCTGCCCAGCAGAAGTATCAGCCTCTG
GACGAGCTGGACAAGACCCTGTACGACCAGTTCATGCTCCAGCAGGGACTGGAAt
ga
SEQ ID NO 57, DNA sequence (DHAMDH02083016) for
2xCon4(C) fused to the C-terminus of the Heavy
Chain of Bevacizumab, with linker peptide
GGGGSGGGGSGGGGS
ATGGGTTGGTCCTGTATCATTCTTTTCCTCGTCGCCACTGCCACCGGAGTCCACT
CAGAAGTCCAGTTGGTGGAGTCGGGAGGAGGACTGGTGCAGCCAGGCGGCTCCCT
GCGCCTGTCCTGCGCGGCGTCCGGGTACACCTTCACCAACTACGGCATGAACTGG
GTCCGCCAGGCCCCCGGAAAGGGGCTGGAATGGGTCGGCTGGATCAACACTTACA
CCGGAGAACCTACCTACGCTGCCGATTTCAAGCGGCGCTTTACTTTCTCGCTGGA
CACCTCCAAGAGCACCGCCTATCTCCAAATGAACTCCCTGCGGGCCGAGGATACC
GCCGTGTACTATTGCGCGAAGTACCCCCACTATTACGGTTCGTCCCATTGGTACT
TCGACGTCTGGGGCCAGGGAACTCTTGTCACTGTGTCCTCCGCATCCACCAAGGG
ACCGTCAGTGTTCCCCCTGGCCCCGTCCTCCAAAAGCACTAGCGGAGGAACCGCA
GCCTTGGGATGCCTCGTCAAGGACTACTTTCCCGAGCCTGTCACCGTGTCGTGGA
ACTCCGGTGCCCTCACTTCGGGCGTGCACACGTTCCCAGCGGTGCTGCAGTCCAG
CGGACTGTACTCGCTGTCCTCCGTCGTGACCGTGCCTTCATCGAGCCTGGGGACC
CAGACCTACATTTGCAACGTGAACCACAAGCCCTCCAACACCAAAGTGGACAAGA
AGGTCGAACCAAAGAGCTGCGACAAGACCCACACTTGCCCGCCGTGCCCGGCCCC
TGAGTTGCTGGGTGGTCCATCGGTGTTCCTGTTCCCGCCTAAGCCGAAGGACACA
CTCATGATCAGCAGGACCCCCGAAGTGACCTGTGTGGTGGTCGACGTGTCACATG
AAGATCCCGAGGTCAAGTTCAATTGGTACGTGGACGGAGTGGAAGTGCATAATGC
CAAGACTAAGCCGAGAGAGGAACAGTACAACTCCACCTACCGGGTGGTGTCAGTG
CTGACCGTGCTCCATCAGGACTGGCTCAACGGGAAGGAGTACAAGTGCAAAGTGT
CGAACAAGGCTCTCCCCGCCCCTATCGAGAAAACCATTAGCAAGGCTAAGGGACA
GCCGCGGGAGCCGCAAGTGTACACCCTGCCCCCGAGCCGCGAAGAAATGACTAAG
AACCAAGTGTCCCTGACCTGTCTCGTGAAAGGGTTCTACCCGTCGGACATCGCTG
TGGAGTGGGAGTCTAATGGTCAACCTGAGAACAACTACAAGACTACTCCCCCTGT
GCTGGACTCCGATGGTTCCTTTTTCCTGTACTCAAAGCTGACCGTGGACAAGTCC
AGATGGCAGCAGGGCAACGTGTTCAGCTGCTCCGTGATGCATGAAGCACTTCACA
ACCACTACACCCAGAAGTCCCTCAGCCTGTCTCCGGGGAAGGGCGGCGGAGGAGG
GGCCCAGCAGGAAGAGTGTGAATGGGACCCCTGGACTTGTGAACACATGGGCGGC
GGCGGCTCCGGTGGAGGAGGATCCGGCGGAGGGGGCAGCGCGACGCACCAGGAGG
AGTGCGAATGGGATCCATGGACTTGCGAACACATGCTGGAGTGA
SEQ ID NO 58, DNA sequence (DHAMDL083016),
for the light chain of Bevacizumab
ATGGGTTGGTCCTGTATTATCCTCTTTCTCGTCGCCACTGCCACCGGAGTGCACT
CAGATATTCAGATGACCCAGAGCCCCTCCTCACTGTCCGCTTCCGTGGGGGACCG
CGTGACTATCACTTGCTCGGCTTCCCAAGATATCTCCAACTACCTGAACTGGTAC
CAGCAGAAGCCCGGAAAGGCCCCGAAAGTGCTCATCTACTTCACCTCATCGCTGC
ACTCGGGAGTGCCCTCAAGATTTTCCGGCTCCGGAAGCGGGACCGACTTCACTCT
TACCATCTCATCGTTGCAACCAGAGGATTTCGCGACCTACTACTGTCAGCAGTAC
TCCACGGTGCCGTGGACCTTCGGACAAGGCACCAAAGTGGAGATCAAGAGGACTG
TGGCGGCCCCGAGCGTGTTCATTTTCCCTCCTTCCGACGAGCAGCTGAAAAGCGG
CACCGCCTCGGTCGTGTGCCTCCTGAACAACTTCTACCCGCGGGAAGCCAAGGTC
CAGTGGAAGGTCGACAACGCGCTGCAGAGCGGAAATTCCCAGGAGAGCGTGACCG
AACAGGACTCCAAGGACAGCACCTATTCCCTGTCGTCTACACTGACCCTGAGCAA
GGCCGACTACGAGAAGCATAAGGTCTACGCATGCGAAGTGACCCACCAAGGTCTT
TCCTCCCCTGTGACCAAGTCCTTCAACCGGGGCGAATGCTGA
SEQ ID NO 59, DNA sequence (LY2.55.1), for peptide
L1-15 (no LE) fused to the N-terminus of the light
chain of Bevacizumab
ATGGCCTGGATGATGTTGCTTCTCGGACTTCTCGCGTATGGATCAGGGGTGGATA
GCGCGCAACAGAAGTACCAGCCTTTGGACGAACTGGACAAGACCCTGTACGACCA
GTTCATGCTGCAACAGGGAGGGGGCGGTGGATCCGGGGGCGGCGGCTCCGGCGGT
GGCGGATCCGACATTCAAATGACTCAGTCGCCATCGTCCCTCTCGGCATCCGTGG
GAGACAGAGTGACCATCACTTGTTCCGCCTCGCAAGACATCTCCAACTACCTGAA
CTGGTACCAGCAGAAGCCCGGGAAGGCCCCCAAAGTGCTCATCTACTTTACTTCC
TCACTGCACTCCGGGGTGCCAAGCCGCTTTAGCGGCTCCGGTTCTGGAACCGATT
TCACCCTGACCATTAGCTCACTCCAGCCGGAAGATTTCGCTACGTACTACTGCCA
GCAGTATTCGACCGTGCCGTGGACTTTCGGACAGGGTACCAAAGTCGAGATCAAG
CGGACCGTGGCCGCCCCGAGCGTGTTCATTTTCCCGCCTTCCGACGAGCAACTCA
AGTCCGGCACTGCCTCCGTGGTCTGCCTGCTGAACAATTTCTACCCCCGCGAGGC
TAAGGTCCAGTGGAAGGTCGATAACGCACTGCAGTCCGGAAACAGCCAAGAGAGC
GTGACCGAACAGGACTCCAAGGACTCAACTTACTCGCTGAGCTCCACCCTGACCC
TGTCGAAGGCCGACTACGAAAAGCACAAAGTGTACGCCTGCGAAGTGACACATCA
GGGCCTGTCATCCCCTGTCACCAAGTCCTTCAACCGGGGAGAGTGCTGATAA
SEQ ID NO 60, DNA sequence (LY2.55.2), for peptide
L1-15 (with LE) fused to the N-terminus of the
light chain of Bevacizumab
ATGGCCTGGATGATGTTGCTTCTCGGACTTCTCGCGTATGGATCAGGGGTGGATA
GCGCGCAACAGAAGTACCAGCCTTTGGACGAACTGGACAAGACCCTGTACGACCA
GTTCATGCTGCAACAGGGACTGGAAGGGGGCGGTGGATCCGGGGGCGGCGGCTCC
GGCGGTGGCGGATCCGACATTCAAATGACTCAGTCGCCATCGTCCCTCTCGGCAT
CCGTGGGAGACAGAGTGACCATCACTTGTTCCGCCTCGCAAGACATCTCCAACTA
CCTGAACTGGTACCAGCAGAAGCCCGGGAAGGCCCCCAAAGTGCTCATCTACTTT
ACTTCCTCACTGCACTCCGGGGTGCCAAGCCGCTTTAGCGGCTCCGGTTCTGGAA
CCGATTTCACCCTGACCATTAGCTCACTCCAGCCGGAAGATTTCGCTACGTACTA
CTGCCAGCAGTATTCGACCGTGCCGTGGACTTTCGGACAGGGTACCAAAGTCGAG
ATCAAGCGGACCGTGGCCGCCCCGAGCGTGTTCATTTTCCCGCCTTCCGACGAGC
AACTCAAGTCCGGCACTGCCTCCGTGGTCTGCCTGCTGAACAATTTCTACCCCCG
CGAGGCTAAGGTCCAGTGGAAGGTCGATAACGCACTGCAGTCCGGAAACAGCCAA
GAGAGCGTGACCGAACAGGACTCCAAGGACTCAACTTACTCGCTGAGCTCCACCC
TGACCCTGTCGAAGGCCGACTACGAAAAGCACAAAGTGTACGCCTGCGAAGTGAC
ACATCAGGGCCTGTCATCCCCTGTCACCAAGTCCTTCAACCGGGGAGAGTGCTGA
TAA
SEQ ID NO 61, DNA sequence (LY2.55.3), for peptide
L1-15 (no LE) fused to the N-terminus of the heavy
chain of Bevacizumab
ATGGCTTGGATGATGCTGCTGCTTGGCCTTCTCGCATACGGTTCCGGAGTCGATA
GCGCCCAACAGAAGTACCAGCCTCTGGACGAACTGGATAAGACCCTGTACGATCA
GTTCATGCTGCAACAGGGGGGCGGCGGAGGATCGGGCGGTGGTGGATCCGGCGGC
GGCGGATCCGAAGTGCAGCTCGTGGAGAGCGGGGGCGGACTCGTGCAGCCGGGAG
GTTCGCTGAGATTGTCCTGTGCCGCCTCCGGTTACACCTTTACCAATTACGGGAT
GAACTGGGTCCGCCAGGCCCCCGGAAAGGGACTGGAATGGGTCGGCTGGATCAAC
ACATATACCGGAGAGCCCACCTACGCCGCGGACTTCAAGCGGAGATTCACCTTTT
CACTGGATACGTCAAAGTCAACTGCATACCTCCAGATGAACTCCCTTAGGGCGGA
AGATACCGCCGTGTACTACTGCGCCAAGTACCCGCACTATTACGGGTCCAGCCAT
TGGTACTTCGACGTCTGGGGACAGGGGACCCTCGTGACCGTCAGCAGCGCCTCCA
CCAAGGGCCCGTCCGTGTTCCCTCTTGCGCCGTCGTCCAAAAGCACTTCCGGCGG
CACTGCCGCCCTGGGCTGCCTCGTGAAGGATTACTTCCCGGAACCGGTCACCGTG
TCGTGGAACTCCGGAGCCCTGACTTCGGGTGTCCACACCTTCCCTGCGGTGCTGC
AGAGCTCCGGTCTGTACTCCCTCTCTTCCGTGGTCACGGTGCCCTCCTCATCACT
GGGAACCCAGACCTACATCTGCAACGTGAACCACAAGCCCTCAAACACTAAGGTC
GACAAGAAAGTCGAACCGAAGTCGTGCGACAAGACCCACACTTGCCCTCCGTGCC
CGGCTCCCGAGCTGCTGGGGGGCCCTTCCGTGTTTTTGTTCCCGCCGAAACCAAA
GGACACTCTGATGATCAGCCGCACTCCGGAAGTGACCTGTGTGGTGGTGGACGTG
TCCCACGAGGACCCAGAAGTGAAATTCAATTGGTACGTGGATGGCGTGGAAGTGC
ACAACGCTAAGACTAAGCCCCGCGAGGAACAGTACAACAGCACTTACCGGGTGGT
GTCGGTGCTCACCGTGCTGCACCAAGATTGGCTCAACGGGAAGGAGTACAAGTGC
AAAGTCTCCAACAAGGCCCTGCCCGCACCTATTGAAAAGACCATCAGCAAGGCCA
AGGGACAGCCCCGGGAGCCCCAGGTCTACACCCTGCCTCCCTCGCGCGAAGAGAT
GACTAAGAACCAAGTGTCCCTGACCTGTCTGGTCAAGGGATTCTATCCTTCCGAC
ATTGCCGTGGAATGGGAGTCCAACGGGCAGCCAGAGAACAACTACAAGACCACTC
CACCTGTGCTGGACTCCGACGGGTCCTTCTTCTTGTACTCGAAGCTGACCGTGGA
CAAGTCCCGGTGGCAGCAGGGAAACGTGTTCAGCTGCTCCGTGATGCACGAGGCC
TTGCATAATCATTACACCCAAAAGTCGCTGAGCTTGAGCCCGGGAAAGTGATAA
SEQ ID NO 62, DNA sequence (LY2.55.4), for peptide
L1-15 (with LE) fused to the N-terminus of the heavy
chain of Bevacizumab
ATGGCTTGGATGATGCTGCTGCTTGGCCTTCTCGCATACGGTTCCGGAGTCGATA
GCGCCCAACAGAAGTACCAGCCTCTGGACGAACTGGATAAGACCCTGTACGATCA
GTTCATGCTGCAACAGGGGCTTGAGGGCGGCGGAGGATCGGGCGGTGGTGGATCC
GGCGGCGGCGGATCCGAAGTGCAGCTCGTGGAGAGCGGGGGCGGACTCGTGCAGC
CGGGAGGTTCGCTGAGATTGTCCTGTGCCGCCTCCGGTTACACCTTTACCAATTA
CGGGATGAACTGGGTCCGCCAGGCCCCCGGAAAGGGACTGGAATGGGTCGGCTGG
ATCAACACATATACCGGAGAGCCCACCTACGCCGCGGACTTCAAGCGGAGATTCA
CCTTTTCACTGGATACGTCAAAGTCAACTGCATACCTCCAGATGAACTCCCTTAG
GGCGGAAGATACCGCCGTGTACTACTGCGCCAAGTACCCGCACTATTACGGGTCC
AGCCATTGGTACTTCGACGTCTGGGGACAGGGGACCCTCGTGACCGTCAGCAGCG
CCTCCACCAAGGGCCCGTCCGTGTTCCCTCTTGCGCCGTCGTCCAAAAGCACTTC
CGGCGGCACTGCCGCCCTGGGCTGCCTCGTGAAGGATTACTTCCCGGAACCGGTC
ACCGTGTCGTGGAACTCCGGAGCCCTGACTTCGGGTGTCCACACCTTCCCTGCGG
TGCTGCAGAGCTCCGGTCTGTACTCCCTCTCTTCCGTGGTCACGGTGCCCTCCTC
ATCACTGGGAACCCAGACCTACATCTGCAACGTGAACCACAAGCCCTCAAACACT
AAGGTCGACAAGAAAGTCGAACCGAAGTCGTGCGACAAGACCCACACTTGCCCTC
CGTGCCCGGCTCCCGAGCTGCTGGGGGGCCCTTCCGTGTTTTTGTTCCCGCCGAA
ACCAAAGGACACTCTGATGATCAGCCGCACTCCGGAAGTGACCTGTGTGGTGGTG
GACGTGTCCCACGAGGACCCAGAAGTGAAATTCAATTGGTACGTGGATGGCGTGG
AAGTGCACAACGCTAAGACTAAGCCCCGCGAGGAACAGTACAACAGCACTTACCG
GGTGGTGTCGGTGCTCACCGTGCTGCACCAAGATTGGCTCAACGGGAAGGAGTAC
AAGTGCAAAGTCTCCAACAAGGCCCTGCCCGCACCTATTGAAAAGACCATCAGCA
AGGCCAAGGGACAGCCCCGGGAGCCCCAGGTCTACACCCTGCCTCCCTCGCGCGA
AGAGATGACTAAGAACCAAGTGTCCCTGACCTGTCTGGTCAAGGGATTCTATCCT
TCCGACATTGCCGTGGAATGGGAGTCCAACGGGCAGCCAGAGAACAACTACAAGA
CCACTCCACCTGTGCTGGACTCCGACGGGTCCTTCTTCTTGTACTCGAAGCTGAC
CGTGGACAAGTCCCGGTGGCAGCAGGGAAACGTGTTCAGCTGCTCCGTGATGCAC
GAGGCCTTGCATAATCATTACACCCAAAAGTCGCTGAGCTTGAGCCCGGGAAAGT
GATAA
SEQ ID NO 63, DNA sequence (LY2.55.5), for the light
chain of Bevacizumab
ATGGCCTGGATGATGTTGCTTCTCGGACTTCTCGCGTATGGATCAGGGGTGGACT
CCGACATTCAAATGACTCAGTCGCCATCGTCCCTCTCGGCATCCGTGGGAGACAG
AGTGACCATCACTTGTTCCGCCTCGCAAGACATCTCCAACTACCTGAACTGGTAC
CAGCAGAAGCCCGGGAAGGCCCCCAAAGTGCTCATCTACTTTACTTCCTCACTGC
ACTCCGGGGTGCCAAGCCGCTTTAGCGGCTCCGGTTCTGGAACCGATTTCACCCT
GACCATTAGCTCACTCCAGCCGGAAGATTTCGCTACGTACTACTGCCAGCAGTAT
TCGACCGTGCCGTGGACTTTCGGACAGGGTACCAAAGTCGAGATCAAGCGGACCG
TGGCCGCCCCGAGCGTGTTCATTTTCCCGCCTTCCGACGAGCAACTCAAGTCCGG
CACTGCCTCCGTGGTCTGCCTGCTGAACAATTTCTACCCCCGCGAGGCTAAGGTC
CAGTGGAAGGTCGATAACGCACTGCAGTCCGGAAACAGCCAAGAGAGCGTGACCG
AACAGGACTCCAAGGACTCAACTTACTCGCTGAGCTCCACCCTGACCCTGTCGAA
GGCCGACTACGAAAAGCACAAAGTGTACGCCTGCGAAGTGACACATCAGGGCCTG
TCATCCCCTGTCACCAAGTCCTTCAACCGGGGAGAGTGCTGATAA
SEQ ID NO 64, 2xCon4(C) fused to the C-terminus of the
VEGF Trap
10 20 30 40 50 60
SDTGRPFVEM YSEIPEIIHM TEGRELVIPC RVTSPNITVT LKKFPLDTLI PDGKRIIWDS
        70 80 90 100 110 120
RKGFIISNAT YKEIGLLTCE ATVNGHLYKT NYLTHRQTNT IIDVVLSPSH GIELSVGEKL
      130 140 150 160 170 180
VLNCTARTEL NVGIDFNWEY PSSKHQHKKL VNRDLKTQSG SEMKKFLSTL TIDGVTRSDQ
      190 200 210 220 230 240
GLYTCAASSG LMTKKNSTFV RVHEKDKTHT CPPCPAPELL GGPSVFLFPP KPKDTLMISR
      250 260 270 280 290 300
TPEVTCVVVD VSHEDPEVKF NWYVDGVEVH NAKTKPREEQ YNSTYRVVSV LTVLHQDWLN
      310 320 330 340 350 360
GKEYKCKVSN KALPAPIEKT ISKAKGQPRE PQVYTLPPSR DELTKNQVSL TCLVKGFYPS
      370 380 390 400 410 420
DIAVEWESNG QPENNYKTTP PVLDSDGSFF LYSKLTVDKS RWQQGNVFSC SVMHEALHNH
      430 440 450 460 470 480
YTQKSLSLSP GKGGGGGAQQ EECEWDPWTC EHMGSGSATG GSGSTASSGS GSATHQEECE
      490
WDPWTCEHML E
```

1. A method of treating a human patient with proliferative retinopathy, wet age-related macular degeneration (wAMD), macular edema following retinal vein occlusion (RVO), diabetic macular edema (DME), or diabetic retinopathy (DR) comprising administering to the patient a pharmaceutical composition comprising a chimeric molecule comprising:
(a) a light chain polypeptide comprising an amino acid sequence that is at least 95% identical to the amino acid sequence set forth in SEQ ID NO: 4, wherein the light chain polypeptide comprises complementary-determining region (CDR) sequences having the amino acid sequences of SASQDISNYLN (SEQ ID NO: 65), FTSSLHS (SEQ ID NO: 66), and QQYSTVPWT (SEQ ID NO: 67); and
(b) a heavy chain polypeptide comprising an amino acid sequence that is at least 99% identical to the amino acid sequence set forth in SEQ ID NO: 31, SEQ ID NO: 32, SEQ ID NO:
34, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53, wherein the heavy chain polypeptide comprises CDR sequences having the amino acid sequences of HYGMN, WINTYTGEPTYAADFKR, and YPYYYGTSHWYFDV.

2. A method of treating a human patient with proliferative retinopathy, wet age-related macular degeneration (wAMD), macular edema following retinal vein occlusion (RVO), diabetic macular edema (DME), or diabetic retinopathy (DR) comprising administering to the patient a pharmaceutical composition comprising a chimeric molecule comprising four polypeptides:
(a) wherein two polypeptides comprise an amino acid sequence that is at least 95% identical to the amino acid sequence set forth in SEQ ID NO: 4 and comprise complementary-determining region (CDR) sequences having the amino acid sequences of SASQDISNYLN (SEQ ID NO: 65), FTSSLHS (SEQ ID NO: 66), and QQYSTVPWT (SEQ ID NO: 67); and
(b) wherein two polypeptides comprise an amino acid sequence that is at least 99% identical to the amino acid sequence set forth in SEQ ID NO: 31, SEQ ID NO: 32, SEQ ID NO: 34, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53 and comprise CDR sequences having the amino acid sequences of HYGMN, WINTYTGEPTYAADFKR, and YPYYYGTSHWYFDV.

3. A method of treating a human patient with proliferative retinopathy, wet age-related macular degeneration (wAMD), macular edema following retinal vein occlusion (RVO), diabetic macular edema (DME), or diabetic retinopathy (DR) comprising administering to the patient a pharmaceutical composition comprising a chimeric molecule comprising one or two VEGF-binding moieties and one or two Ang-2 antagonist peptides, wherein:
(a) the Ang-2 antagonist peptide comprises an amino acid sequence selected from the amino acid sequences of SEQ ID NO: 8-14; and
(b) the VEGF-binding moiety is an antibody or a Fab, wherein the antibody or the Fab comprises:
(i) a light chain polypeptide comprising an amino acid sequence that is at least 95% identical to the amino acid sequence set forth in SEQ ID NO: 4, wherein the light chain polypeptide comprises complementary-determining region (CDR) sequences having the amino acid sequences of SASQDISNYLN (SEQ ID NO: 65), FTSSLHS (SEQ ID NO: 66), and QQYSTVPWT (SEQ ID NO: 67), and
(ii) a heavy chain polypeptide comprising CDR sequences having the amino acid sequences of HYGMN, WINTYTGEPTYAADFKR, and YPYYYGTSHWYFDV.

4. The method of claim 3, wherein:
the light chain polypeptide comprises the amino acid sequence set forth in SEQ ID NO: 4; and
the heavy chain polypeptide comprises the amino acid sequence set forth in SEQ ID NO: 31.

5. The method of claim 3, wherein:
the light chain polypeptide comprises the amino acid sequence set forth in SEQ ID NO: 4; and
the heavy chain polypeptide comprises the amino acid sequence set forth in SEQ ID NO: 25 or SEQ ID NO: 26.


SEQUENCE LISTING (70 sequences)

SEQ ID NO: 1 - protein, 453 aa, synthetic construct
  REGION 1..453: note = Bevacizumab Heavy Chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  REGION 1..214: note = Bevacizumab Light Chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 432 aa, synthetic construct
  REGION 1..432: note = VEGF Trap Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
  REGION 1..214: note = Protein Sequence for Light Chain, Ranibizumab (VEGF Fab)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 231 aa, synthetic construct
  REGION 1..231: note = Heavy Chain Ranibizumab (VEGF Fab)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 6 - protein, 251 aa, synthetic construct
  REGION 1..251: note = VEGF ScFv
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 7 - protein, 453 aa, synthetic construct
  REGION 1..453: note = heavy chain of a VEGF antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 25 aa, synthetic construct
  REGION 1..25: note = L1-7
AQQTNFMPMDDLEQRLYEQFILQQG

SEQ ID NO: 9 - protein, 24 aa, synthetic construct
  REGION 1..24: note = L1-10
AQQKFQPLDELEQTLYEQFMLQQA

SEQ ID NO: 10 - protein, 24 aa, synthetic construct
  REGION 1..24: note = L1-15
AQQKYQPLDELDKTLYDQFMLQQG

SEQ ID NO: 11 - protein, 23 aa, synthetic construct
  REGION 1..23: note = L1-7B
QTNFMPMDDLEQRLYEQFILQQG

SEQ ID NO: 12 - protein, 22 aa, synthetic construct
  REGION 1..22: note = L1-10B
QKFQPLDELEQTLYEQFMLQQA

SEQ ID NO: 13 - protein, 22 aa, synthetic construct
  REGION 1..22: note = L1-15B
QKYQPLDELDKTLYDQFMLQQG

SEQ ID NO: 14 - protein, 22 aa, synthetic construct
  REGION 1..22: note = CVX-060
QKYQPLDEKDKTLYDQFMLQQG

SEQ ID NO: 15 - protein, 471 aa, synthetic construct
  REGION 1..471: note = L1-15 fused to the N-terminus of the VEGF Trap, with linker peptide GGGGSGGGGSGGGGS
AQQKYQPLDELDKTLYDQFMLQQGGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 464 aa, synthetic construct
  REGION 1..464: note = AMD-I (L1-15 fused to VEGF Trap)
  VARIANT 1: note = X is A, G, or deleted
  VARIANT 2: note = X is Q or A or deleted
  VARIANT 5: note = X is Y or F
  VARIANT 12: note = X is D or E
  VARIANT 13: note = X is Q or K
  VARIANT 17: note = X is D or E
  VARIANT 25: note = X is L or deleted
  VARIANT 26: note = X is E is deleted
  VARIANT 27..31: note = Sequence can be deleted or repeated such that (GGGGS)n wherein n = 0, 1, 2, 3, 4, or 5
  VARIANT 464: note = C-terminal amino acid K may be deleted
XXQKXQPLDELXXTLYXQFMLQQGXXGGGGSNSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 464 aa, synthetic construct
  REGION 1..464: note = AMD-J (L1-7 fused to VEGF Trap)
  SITE 1: note = variant - X is A, G, or deleted
  SITE 2: note = variant - X is Q or A or deleted
  SITE 26: note = variant - X is L or deleted
  SITE 27: note = variant - X is E is deleted
  REGION 28..32: note = variant - (GGGGS)n can be repeated such that n = 0, 1, 2, 3, 4, or 5
  SITE 464: note = variant - C-terminal amino acid K may be deleted
XXQTNFMPMDDLEQRLYEQFILQQGXXGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - skipped

SEQ ID NO: 19 - protein, 262 aa, synthetic construct
  REGION 1..262: note = AMD-K Heavy Chain (L1-15 fused to VEGF Fab)
  SITE 1: note = variant - X is A, G, or deleted
  SITE 2: note = variant - X is Q or A or deleted
  SITE 5: note = variant - X is Y or F
  SITE 12: note = variant - X is D or E
  SITE 13: note = variant - X is Q or K
  SITE 17: note = variant - X is D or E
  SITE 25: note = variant - X is L or deleted
  SITE 26: note = variant - X is E is deleted
  REGION 27..31: note = variant - Sequence can be deleted or repeated such that (GGGGS)n wherein n = 0, 1, 2, 3, 4, or 5
XXQKXQPLDELXXTLYXQFMLQQGXXGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 20 - protein, 263 aa, synthetic construct
  REGION 1..263: note = L1-7 fused to VEGF Fab
  SITE 1: note = variant - X is A, G, or deleted
  SITE 2: note = variant - X is Q or A or deleted
  SITE 26: note = variant - X is L or deleted
  SITE 27: note = variant - X is E or deleted
  REGION 28..32: note = variant - Sequence can be deleted or repeated such that (GGGGS)n wherein n = 0, 1, 2, 3, 4, or 5
XXQTNFMPMDDLEQRLYEQFILQQGXXGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 21 - protein, 282 aa, synthetic construct
  REGION 1..282: note = L1-15 fused to VEGF ScFv
  SITE 1: note = variant - X is A, G, or deleted
  SITE 2: note = variant - X is Q or A or deleted
  SITE 5: note = variant - X is Y or F
  SITE 12: note = variant - X is D or E
  SITE 13: note = variant - X is Q or K
  SITE 17: note = variant - X is D or E
  SITE 25: note = variant - X is L or deleted
  SITE 26: note = variant - X is E is deleted
  REGION 27..31: note = variant - Sequence can be deleted or repeated such that (GGGGS)n wherein n = 0, 1, 2, 3, 4, or 5
XXQKXQPLDELXXTLYXQFMLQQGXXGGGGSEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 22 - protein, 283 aa, synthetic construct
  REGION 1..283: note = L1-7 fused to VEGF ScFv
  SITE 1: note = variant - X is A, G, or deleted
  SITE 2: note = variant - X is Q or A or deleted
  SITE 26: note = variant - X is L or deleted
  SITE 27: note = variant - X is E is deleted
  REGION 28..32: note = variant - Sequence can be deleted or repeated such that (GGGGS)n wherein n = 0, 1, 2, 3, 4, or 5
XXQTNFMPMDDLEQRLYEQFILQQGXXGGGGSEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 23 - protein, 463 aa, synthetic construct
  REGION 1..463: note = L1-15 fused to C-terminal of VEGF Trap
  REGION 433..437: note = variant - (GGGGS)n can be absent or repeated, wherein n = 0, 1, 2, 3, 4, or 5
  SITE 438: note = variant - X is A, G, or deleted
  SITE 439: note = variant - X is Q or A or deleted
  SITE 440: note = variant - X is Q or A or deleted
  SITE 442: note = variant - X is Y or F
  SITE 449: note = variant - X is D or E
  SITE 450: note = variant - X is Q or K
  SITE 454: note = variant - X is D or E
  SITE 462: note = variant - X is L or deleted
  SITE 463: note = variant - X is E or deleted
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSXXQKXQPLDELXXTLYXQFMLQQGXX

SEQ ID NO: 24 - protein, 464 aa, synthetic construct
  REGION 1..464: note = L1-7 fused to C-terminal of VEGF Trap
  REGION 433..437: note = variant - Sequence can be deleted or repeated such that (GGGGS)n wherein n = 0, 1, 2, 3, 4, or 5
  SITE 438: note = variant - X is A, G, or deleted
  SITE 439: note = variant - X is Q, A or deleted
  SITE 463: note = variant - X is L or deleted
  SITE 464: note = variant - X is E or deleted
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSXXQTNFMPMDDLEQRLYEQFILQQGXX

SEQ ID NO: 25 - protein, 262 aa, synthetic construct
  REGION 1..262: note = L1-15 fused to C-terminal VEGF Fab
  SITE 237: note = variant - X is A, G, or deleted
  SITE 238: note = variant - X is Q or A or deleted
  SITE 241: note = variant - X is Y or F
  SITE 248: note = variant - X is D or E
  SITE 249: note = variant - X is Q or K
  SITE 253: note = variant - X is D or E
  SITE 261: note = variant - X is L or deleted
  SITE 262: note = variant - X is E is deleted
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGGGGSXXQKXQPLDELXXTLYXQFMLQQGXX

SEQ ID NO: 26 - protein, 263 aa, synthetic construct
  REGION 1..263: note = L1-7 fused to VEGF Fab
  REGION 232..236: note = variant - Sequence can be deleted or repeated such that (GGGGS)n wherein n = 0, 1, 2, 3, 4, or 5
  SITE 237: note = variant - X is A, G, or deleted
  SITE 238: note = variant - X is Q or A or deleted
  SITE 262: note = variant - X is L or deleted
  SITE 263: note = variant - X is E is deleted
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGGGGSXXQTNFMPMDDLEQRLYEQFILQQGXX

SEQ ID NO: 27 - protein, 282 aa, synthetic construct
  REGION 1..282: note = L1-15 fused to C-terminal VEGF ScFv
  REGION 252..256: note = variant - Sequence can be deleted or repeated such that (GGGGS)n wherein n = 0, 1, 2, 3, 4, or 5
  SITE 257: note = variant - X is A, G, or deleted
  SITE 258: note = variant - X is Q or A or deleted
  SITE 261: note = variant - X is Y or F
  SITE 268: note = variant - X is D or E
  SITE 269: note = variant - X is Q or K
  SITE 273: note = variant - X is D or E
  SITE 281: note = variant - X is L or deleted
  SITE 282: note = variant - X is E is deleted
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSGGGGSXXQKXQPLDELXXTLYXQFMLQQGXX

SEQ ID NO: 28 - protein, 283 aa, synthetic construct
  REGION 1..283: note = AMD-Q-C terminal (L1-7 fused to VEGF ScFv)
  REGION 252..256: note = variant - Sequence can be deleted or repeated such that (GGGGS)n wherein n = 0, 1, 2, 3, 4, or 5
  REGION 257..258: note = misc_feature - X can be any naturally occurring amino acid
  SITE 266: note = variant - X is A, G, or deleted
  SITE 267: note = variant - X is Q or A or deleted
  SITE 282: note = variant - X is L or deleted
  SITE 283: note = variant - X is E is deleted
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSGGGGSXXQTNFMPMDDLEQRLYEQFILQQGXX

SEQ ID NO: 29 - protein, 494 aa, synthetic construct
  REGION 1..494: note = ASKB712-O (L1-15 fused to the N-terminal of an VEGF-binding antibody)
AQQKYQPLDELDKTLYDQFMLQQGLEGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 484 aa, synthetic construct
  REGION 1..484: note = ASKB712-O3 (L1-15 fused to the N-terminal of an VEGF-binding antibody)
  SITE 1: note = variant - X is A, G, or deleted
  SITE 2: note = variant - X is Q or A or deleted
  SITE 5: note = variant - X is Y or F
  SITE 12: note = variant - X is D or E
  SITE 13: note = variant - X is Q or K
  SITE 17: note = variant - X is D or E
  SITE 25: note = variant - X is L or deleted
  SITE 26: note = variant - X is E is deleted
  REGION 27..31: note = variant - Sequence can be deleted or repeated such that (GGGGS)n wherein n = 0, 1, 2, 3, 4, or 5
XXQKXQPLDELXXTLYXQFMLQQGXXGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 494 aa, synthetic construct
  REGION 1..494: note = ASKB712-O2 (L1-15 fused to the C-terminal of an VEGF-binding antibody)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAQQKYQPLDELDKTLYDQFMLQQGLE

SEQ ID NO: 32 - protein, 484 aa, synthetic construct
  REGION 1..484: note = ASKB712-O4 (L1-15 fused to the C-terminal of an VEGF-binding antibody)
  REGION 454..458: note = variant - Sequence can be deleted or repeated such that (GGGGS)n wherein n = 0, 1, 2, 3, 4, or 5
  SITE 459: note = variant - X is A, G, or deleted
  SITE 460: note = variant - X is Q or A or deleted
  SITE 463: note = variant - X is Y or F
  SITE 470: note = variant - X is D or E
  SITE 471: note = variant - X is Q or K
  SITE 475: note = variant - X is D or E
  SITE 483: note = variant - X is L or deleted
  SITE 484: note = variant - X is E is deleted
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSXXQKXQPLDELXXTLYXQFMLQQGXX

SEQ ID NO: 33 - protein, 485 aa, synthetic construct
  REGION 1..485: note = ASKB712-P (L1-7 fused to the N-terminal of an VEGF-binding antibody)
  SITE 1: note = variant - X is A, G, or deleted
  SITE 2: note = variant - X is Q or A or deleted
  SITE 26: note = variant - X is L or deleted
  SITE 27: note = variant - X is E is deleted
  REGION 28..32: note = variant - Sequence can be deleted or repeated such that (GGGGS)n wherein n = 0, 1, 2, 3, 4, or 5
XXQTNFMPMDDLEQRLYEQFILQQGXXGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 485 aa, synthetic construct
  REGION 1..485: note = L1-7 fused to the C-terminal of an VEGF-binding antibody
  REGION 454..458: note = variant - Sequence can be deleted or repeated such that (GGGGS)n wherein n = 0, 1, 2, 3, 4, or 5
  SITE 459: note = variant - X is A, G, or deleted
  SITE 460: note = variant - X is Q or A or deleted
  SITE 484: note = variant - X is L or deleted
  SITE 485: note = variant - X is E is deleted
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSXXQTNFMPMDDLEQRLYEQFILQQGXX

SEQ ID NO: 35 - DNA, 1542 bp, synthetic construct
  misc_feature 1..1542: note = 712O_L7_3xGS_DNA sequence
  source 1..1542: mol_type = other DNA
atggaattcggcctgtcttggctgttcctggtggccattctgaagggcgctctggccgctcagaccaacttcatgcctatggacgacctggaacagcggctgtacgagcagttcatcctgcagcaaggactggaaggcggcggaggatctggcggaggcggtagcggaggcggtggatctgaagtgcagctggttgaaagtggcggcggattggttcagcctggcggatctctgagactgtcttgtgccgcctctggctacgacttcacccactacggcatgaattgggtccgacaggctcccggcaaaggcctggaatgggtcggatggatcaacacctataccggcgagcctacctacgccgccgatttcaagcggagattcaccttctccctggacacctccaagtctaccgcctacctgcagatgaactccctgagagccgaggacaccgccgtgtactactgcgctaagtacccctactactacggcaccagccactggtacttcgacgtgtggggacagggcacactggtcacagtgtcctccgcctctaccaagggaccctctgtgtttcctctggctccctccagcaagtccacctctggtggaacagctgctctgggctgcctggtcaaggactactttcctgagcctgtgaccgtgtcctgggcttctggtgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagaaggtggaacccaagtcctgcgataagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagccgggaagagatgaccaagaaccaggtgtccctgacctgcctcgtgaagggcttctacccttccgatatcgccgtggaatgggagagcaatggccagcctgagaacaactacaagaccactcctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtaa

SEQ ID NO: 36 - DNA, 1527 bp, synthetic construct
  misc_feature 1..1527: note = 712O_L7_2xGS_DNA sequence
  source 1..1527: mol_type = other DNA
atggaattcggcctgtcttggctgttcctggtggccattctgaagggcgctctggccgctcagaccaacttcatgcctatggacgacctggaacagcggctgtacgagcagttcatcctgcagcaaggactggaaggcggcggaggatctggaggcggtggatctgaagtgcagctggttgaaagtggcggcggattggttcagcctggcggatctctgagactgtcttgtgccgcctctggctacgacttcacccactacggcatgaattgggtccgacaggctcccggcaaaggcctggaatgggtcggatggatcaacacctataccggcgagcctacctacgccgccgatttcaagcggagattcaccttctccctggacacctccaagtctaccgcctacctgcagatgaactccctgagagccgaggacaccgccgtgtactactgcgctaagtacccctactactacggcaccagccactggtacttcgacgtgtggggacagggcacactggtcacagtgtcctccgcctctaccaagggaccctctgtgtttcctctggctccctccagcaagtccacctctggtggaacagctgctctgggctgcctggtcaaggactactttcctgagcctgtgaccgtgtcctgggcttctggtgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagaaggtggaacccaagtcctgcgataagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagccgggaagagatgaccaagaaccaggtgtccctgacctgcctcgtgaagggcttctacccttccgatatcgccgtggaatgggagagcaatggccagcctgagaacaactacaagaccactcctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtaa

SEQ ID NO: 37 - protein, 508 aa, synthetic construct
  REGION 1..508: note = 712O_L7_2xGS_Protein sequence
MEFGLSWLFLVAILKGALAAQTNFMPMDDLEQRLYEQFILQQGLEGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 1512 bp, synthetic construct
  misc_feature 1..1512: note = 712O_L7_1xGS_DNA sequence
  source 1..1512: mol_type = other DNA
atggaattcggcctgtcttggctgttcctggtggccattctgaagggcgctctggccgctcagaccaacttcatgcctatggacgacctggaacagcggctgtacgagcagttcatcctgcagcaaggactggaaggaggcggtggatctgaagtgcagctggttgaaagtggcggcggattggttcagcctggcggatctctgagactgtcttgtgccgcctctggctacgacttcacccactacggcatgaattgggtccgacaggctcccggcaaaggcctggaatgggtcggatggatcaacacctataccggcgagcctacctacgccgccgatttcaagcggagattcaccttctccctggacacctccaagtctaccgcctacctgcagatgaactccctgagagccgaggacaccgccgtgtactactgcgctaagtacccctactactacggcaccagccactggtacttcgacgtgtggggacagggcacactggtcacagtgtcctccgcctctaccaagggaccctctgtgtttcctctggctccctccagcaagtccacctctggtggaacagctgctctgggctgcctggtcaaggactactttcctgagcctgtgaccgtgtcctgggcttctggtgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagaaggtggaacccaagtcctgcgataagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagccgggaagagatgaccaagaaccaggtgtccctgacctgcctcgtgaagggcttctacccttccgatatcgccgtggaatgggagagcaatggccagcctgagaacaactacaagaccactcctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtaa

SEQ ID NO: 39 - protein, 503 aa, synthetic construct
  REGION 1..503: note = 712O_L7_1xGS
MEFGLSWLFLVAILKGALAAQTNFMPMDDLEQRLYEQFILQQGLEGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1542 bp, synthetic construct
  misc_feature 1..1542: note = 712O_L10_3xGS_DNA sequence
  source 1..1542: mol_type = other DNA
atggaattcggcctgtcttggctgttcctggtggccattctgaagggcgctttggccgctcagcagaagtttcagcctctggacgagctggaacagaccctgtacgagcagttcatgctccagcaggctttggaaggcggcggaggatctggcggaggcggtagcggaggcggtggatctgaagtgcagctggttgaaagtggcggcggattggttcagcctggcggatctctgagactgtcttgtgccgcctctggctacgacttcacccactacggcatgaattgggtccgacaggctcccggcaaaggcctggaatgggtcggatggatcaacacctataccggcgagcctacctacgccgccgatttcaagcggagattcaccttctccctggacacctccaagtctaccgcctacctgcagatgaactccctgagagccgaggacaccgccgtgtactactgcgctaagtacccctactactacggcaccagccactggtacttcgacgtgtggggacagggcacactggtcacagtgtcctccgcctctaccaagggaccctctgtgtttcctctggctccctccagcaagtccacctctggtggaacagctgctctgggctgcctggtcaaggactactttcctgagcctgtgaccgtgtcctgggcttctggtgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagaaggtggaacccaagtcctgcgataagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagccgggaagagatgaccaagaaccaggtgtccctgacctgcctcgtgaagggcttctacccttccgatatcgccgtggaatgggagagcaatggccagcctgagaacaactacaagaccactcctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtaa

SEQ ID NO: 41 - protein, 513 aa, synthetic construct
  REGION 1..513: note = 712O_L10_3xGS_Protein sequence
MEFGLSWLFLVAILKGALAAQQKFQPLDELEQTLYEQFMLQQALEGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1527 bp, synthetic construct
  misc_feature 1..1527: note = 712O_L10_2xGS_DNA sequence
  source 1..1527: mol_type = other DNA
atggaattcggcctgtcttggctgttcctggtggccattctgaagggcgctttggccgctcagcagaagtttcagcctctggacgagctggaacagaccctgtacgagcagttcatgctccagcaggctttggaaggcggcggaggatctggaggcggtggatctgaagtgcagctggttgaaagtggcggcggattggttcagcctggcggatctctgagactgtcttgtgccgcctctggctacgacttcacccactacggcatgaattgggtccgacaggctcccggcaaaggcctggaatgggtcggatggatcaacacctataccggcgagcctacctacgccgccgatttcaagcggagattcaccttctccctggacacctccaagtctaccgcctacctgcagatgaactccctgagagccgaggacaccgccgtgtactactgcgctaagtacccctactactacggcaccagccactggtacttcgacgtgtggggacagggcacactggtcacagtgtcctccgcctctaccaagggaccctctgtgtttcctctggctccctccagcaagtccacctctggtggaacagctgctctgggctgcctggtcaaggactactttcctgagcctgtgaccgtgtcctgggcttctggtgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagaaggtggaacccaagtcctgcgataagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagccgggaagagatgaccaagaaccaggtgtccctgacctgcctcgtgaagggcttctacccttccgatatcgccgtggaatgggagagcaatggccagcctgagaacaactacaagaccactcctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtaa

SEQ ID NO: 43 - protein, 508 aa, synthetic construct
  REGION 1..508: note = 712O_L10_2xGS_Protein sequence
MEFGLSWLFLVAILKGALAAQQKFQPLDELEQTLYEQFMLQQALEGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 1512 bp, synthetic construct
  misc_feature 1..1512: note = 712O_L10_3xGS_DNA sequence
  source 1..1512: mol_type = other DNA
atggaattcggcctgtcttggctgttcctggtggccattctgaagggcgctttggccgctcagcagaagtttcagcctctggacgagctggaacagaccctgtacgagcagttcatgctccagcaggctttggaaggaggcggtggatctgaagtgcagctggttgaaagtggcggcggattggttcagcctggcggatctctgagactgtcttgtgccgcctctggctacgacttcacccactacggcatgaattgggtccgacaggctcccggcaaaggcctggaatgggtcggatggatcaacacctataccggcgagcctacctacgccgccgatttcaagcggagattcaccttctccctggacacctccaagtctaccgcctacctgcagatgaactccctgagagccgaggacaccgccgtgtactactgcgctaagtacccctactactacggcaccagccactggtacttcgacgtgtggggacagggcacactggtcacagtgtcctccgcctctaccaagggaccctctgtgtttcctctggctccctccagcaagtccacctctggtggaacagctgctctgggctgcctggtcaaggactactttcctgagcctgtgaccgtgtcctgggcttctggtgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagaaggtggaacccaagtcctgcgataagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagccgggaagagatgaccaagaaccaggtgtccctgacctgcctcgtgaagggcttctacccttccgatatcgccgtggaatgggagagcaatggccagcctgagaacaactacaagaccactcctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtaa

SEQ ID NO: 45 - protein, 503 aa, synthetic construct
  REGION 1..503: note = 712O_L10_1xGS_Protein sequence
MEFGLSWLFLVAILKGALAAQQKFQPLDELEQTLYEQFMLQQALEGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1512 bp, synthetic construct
  misc_feature 1..1512: note = 712O_C terminal L7_1xGS_DNA sequence
  source 1..1512: mol_type = other DNA
atggaattcggcctgtcttggctgttcctggtggccattctgaagggcgctctggccgaagtgcagttggttgaatctggtggcggattggtgcagcctggcggatctctgagactgtcttgtgccgcctctggctacgatttcacccactacggcatgaattgggtccgacaggctcctggcaaaggcctggaatgggtcggatggatcaatacctataccggcgagcctacctacgccgccgacttcaagagaagattcaccttctccctggacacctccaagtctaccgcctacctgcagatgaactccctgagagctgaggacaccgccgtgtactactgcgctaagtacccctactactacggcaccagccactggtactttgatgtgtggggacagggcaccctggtcaccgtttcttccgcttctacaaagggacccagcgtgttccctctggctcctagctctaagtctacctctggcggaaccgctgctctgggctgtctggtcaaggattacttccctgagcctgtgaccgtgtcctggaatagtggtgctctgacatccggcgtgcacacctttccagctgtgctgcagtcctctggcctgtactctctgtcctctgtcgtgaccgtcccttctagctctctgggcacccagacctacatctgcaacgtgaaccacaagccttccaacactaaggtggacaagaaggtggaacccaagtcctgcgataagacccacacctgtcctccatgtcctgcacctgaagctgctggcggaccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggctaagggccagcctcgggaacctcaggtttacacactgcctccaagccgggaagagatgaccaagaatcaggtgtccctgacctgcctcgtgaagggcttctacccttccgatatcgccgtcgaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtcactctccctttccccgggcgctggcggcggaggatctgctcagaccaacttcatgcctatggacgacctggaacagcggctgtacgagcagttcatcctgcagcaaggactggaatga

SEQ ID NO: 47 - protein, 503 aa, synthetic construct
  REGION 1..503: note = 712O_C terminal L7_1xGS_Protein sequence
MEFGLSWLFLVAILKGALAEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSAQTNFMPMDDLEQRLYEQFILQQGLE

SEQ ID NO: 48 - DNA, 1527 bp, synthetic construct
  misc_feature 1..1527: note = 712O_L7_C_2xGS_DNA sequence
  source 1..1527: mol_type = other DNA
atggaattcggcctgtcttggctgttcctggtggccattctgaagggcgctctggccgaagtgcagttggttgaatctggtggcggattggtgcagcctggcggatctctgagactgtcttgtgccgcctctggctacgatttcacccactacggcatgaattgggtccgacaggctcctggcaaaggcctggaatgggtcggatggatcaatacctataccggcgagcctacctacgccgccgacttcaagagaagattcaccttctccctggacacctccaagtctaccgcctacctgcagatgaactccctgagagctgaggacaccgccgtgtactactgcgctaagtacccctactactacggcaccagccactggtactttgatgtgtggggacagggcaccctggtcaccgtttcttccgcttctacaaagggacccagcgtgttccctctggctcctagctctaagtctacctctggcggaaccgctgctctgggctgtctggtcaaggattacttccctgagcctgtgaccgtgtcctggaatagtggtgctctgacatccggcgtgcacacctttccagctgtgctgcagtcctctggcctgtactctctgtcctctgtcgtgaccgtcccttctagctctctgggcacccagacctacatctgcaacgtgaaccacaagccttccaacactaaggtggacaagaaggtggaacccaagtcctgcgataagacccacacctgtcctccatgtcctgcacctgaagctgctggcggaccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggctaagggccagcctcgggaacctcaggtttacacactgcctccaagccgggaagagatgaccaagaatcaggtgtccctgacctgcctcgtgaagggcttctacccttccgatatcgccgtcgaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtcactctccctttccccgggcgctggcggcggaggatctggcggaggcggtagcgctcagaccaacttcatgcctatggacgacctggaacagcggctgtacgagcagttcatcctgcagcaaggactggaatga

SEQ ID NO: 49 - protein, 508 aa, synthetic construct
  REGION 1..508: note = 712O_L7_C_2xGS_protein sequence
MEFGLSWLFLVAILKGALAEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSAQTNFMPMDDLEQRLYEQFILQQGLE

SEQ ID NO: 50 - DNA, 1542 bp, synthetic construct
  misc_feature 1..1542: note = 712O_L7_C_3xGS_DNA sequence
  source 1..1542: mol_type = other DNA
atggaattcggcctgtcttggctgttcctggtggccattctgaagggcgctctggccgaagtgcagttggttgaatctggtggcggattggtgcagcctggcggatctctgagactgtcttgtgccgcctctggctacgatttcacccactacggcatgaattgggtccgacaggctcctggcaaaggcctggaatgggtcggatggatcaatacctataccggcgagcctacctacgccgccgacttcaagagaagattcaccttctccctggacacctccaagtctaccgcctacctgcagatgaactccctgagagctgaggacaccgccgtgtactactgcgctaagtacccctactactacggcaccagccactggtactttgatgtgtggggacagggcaccctggtcaccgtttcttccgcttctacaaagggacccagcgtgttccctctggctcctagctctaagtctacctctggcggaaccgctgctctgggctgtctggtcaaggattacttccctgagcctgtgaccgtgtcctggaatagtggtgctctgacatccggcgtgcacacctttccagctgtgctgcagtcctctggcctgtactctctgtcctctgtcgtgaccgtcccttctagctctctgggcacccagacctacatctgcaacgtgaaccacaagccttccaacactaaggtggacaagaaggtggaacccaagtcctgcgataagacccacacctgtcctccatgtcctgcacctgaagctgctggcggaccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggctaagggccagcctcgggaacctcaggtttacacactgcctccaagccgggaagagatgaccaagaatcaggtgtccctgacctgcctcgtgaagggcttctacccttccgatatcgccgtcgaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtcactctccctttccccgggcgctggcggcggaggatctggcggaggcggtagcggtggtggtggatctgctcagaccaacttcatgcctatggacgacctggaacagcggctgtacgagcagttcatcctgcagcaaggactggaatga

SEQ ID NO: 51 - protein, 513 aa, synthetic construct
  REGION 1..513: note = 712O_L7_C_3xGS_Protein sequence
MEFGLSWLFLVAILKGALAEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAQTNFMPMDDLEQRLYEQFILQQGLE

SEQ ID NO: 52 - DNA, 1512 bp, synthetic construct
  misc_feature 1..1512: note = 712O_L15_C_1xGS_DNA sequence
  source 1..1512: mol_type = other DNA
atggaattcggcctgtcttggctgttcctggtggccattctgaagggcgctctggccgaagtgcagttggttgaatctggtggcggattggtgcagcctggcggatctctgagactgtcttgtgccgcctctggctacgatttcacccactacggcatgaattgggtccgacaggctcctggcaaaggcctggaatgggtcggatggatcaatacctataccggcgagcctacctacgccgccgacttcaagagaagattcaccttctccctggacacctccaagtctaccgcctacctgcagatgaactccctgagagctgaggacaccgccgtgtactactgcgctaagtacccctactactacggcaccagccactggtactttgatgtgtggggacagggcaccctggtcaccgtttcttccgcttctacaaagggacccagcgtgttccctctggctcctagctctaagtctacctctggcggaaccgctgctctgggctgtctggtcaaggattacttccctgagcctgtgaccgtgtcctggaatagtggtgctctgacatccggcgtgcacacctttccagctgtgctgcagtcctctggcctgtactctctgtcctctgtcgtgaccgtcccttctagctctctgggcacccagacctacatctgcaacgtgaaccacaagccttccaacactaaggtggacaagaaggtggaacccaagtcctgcgataagacccacacctgtcctccatgtcctgcacctgaagctgctggcggaccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggctaagggccagcctcgggaacctcaggtttacacactgcctccaagccgggaagagatgaccaagaatcaggtgtccctgacctgcctcgtgaagggcttctacccttccgatatcgccgtcgaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtcactctccctttccccgggcgctggcggcggaggatctgcccagcagaagtatcagcctctggacgagctggacaagaccctgtacgaccagttcatgctccagcagggactggaatga

SEQ ID NO: 53 - protein, 503 aa, synthetic construct
  REGION 1..503: note = 712O_L15_C_1xGS_Protein sequence
MEFGLSWLFLVAILKGALAEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSAQQKYQPLDELDKTLYDQFMLQQGLE

SEQ ID NO: 54 - DNA, 1526 bp, synthetic construct
  misc_feature 1..1526: note = 712O_L15_C_2xGS_DNA sequence
  source 1..1526: mol_type = other DNA
tggaattcggcctgtcttggctgttcctggtggccattctgaagggcgctctggccgaagtgcagttggttgaatctggtggcggattggtgcagcctggcggatctctgagactgtcttgtgccgcctctggctacgatttcacccactacggcatgaattgggtccgacaggctcctggcaaaggcctggaatgggtcggatggatcaatacctataccggcgagcctacctacgccgccgacttcaagagaagattcaccttctccctggacacctccaagtctaccgcctacctgcagatgaactccctgagagctgaggacaccgccgtgtactactgcgctaagtacccctactactacggcaccagccactggtactttgatgtgtggggacagggcaccctggtcaccgtttcttccgcttctacaaagggacccagcgtgttccctctggctcctagctctaagtctacctctggcggaaccgctgctctgggctgtctggtcaaggattacttccctgagcctgtgaccgtgtcctggaatagtggtgctctgacatccggcgtgcacacctttccagctgtgctgcagtcctctggcctgtactctctgtcctctgtcgtgaccgtcccttctagctctctgggcacccagacctacatctgcaacgtgaaccacaagccttccaacactaaggtggacaagaaggtggaacccaagtcctgcgataagacccacacctgtcctccatgtcctgcacctgaagctgctggcggaccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggctaagggccagcctcgggaacctcaggtttacacactgcctccaagccgggaagagatgaccaagaatcaggtgtccctgacctgcctcgtgaagggcttctacccttccgatatcgccgtcgaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtcactctccctttccccgggcgctggcggcggaggatctggcggaggcggtagcgcccagcagaagtatcagcctctggacgagctggacaagaccctgtacgaccagttcatgctccagcagggactggaatga

SEQ ID NO: 55 - protein, 508 aa, synthetic construct
  REGION 1..508: note = 712O_L15_C_2xGS_Protein sequence
MEFGLSWLFLVAILKGALAEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSAQQKYQPLDELDKTLYDQFMLQQGLE

SEQ ID NO: 56 - DNA, 1542 bp, synthetic construct
  misc_feature 1..1542: note = 712O_L15_C_3xGS_DNA sequence
  source 1..1542: mol_type = other DNA
atggaattcggcctgtcttggctgttcctggtggccattctgaagggcgctctggccgaagtgcagttggttgaatctggtggcggattggtgcagcctggcggatctctgagactgtcttgtgccgcctctggctacgatttcacccactacggcatgaattgggtccgacaggctcctggcaaaggcctggaatgggtcggatggatcaatacctataccggcgagcctacctacgccgccgacttcaagagaagattcaccttctccctggacacctccaagtctaccgcctacctgcagatgaactccctgagagctgaggacaccgccgtgtactactgcgctaagtacccctactactacggcaccagccactggtactttgatgtgtggggacagggcaccctggtcaccgtttcttccgcttctacaaagggacccagcgtgttccctctggctcctagctctaagtctacctctggcggaaccgctgctctgggctgtctggtcaaggattacttccctgagcctgtgaccgtgtcctggaatagtggtgctctgacatccggcgtgcacacctttccagctgtgctgcagtcctctggcctgtactctctgtcctctgtcgtgaccgtcccttctagctctctgggcacccagacctacatctgcaacgtgaaccacaagccttccaacactaaggtggacaagaaggtggaacccaagtcctgcgataagacccacacctgtcctccatgtcctgcacctgaagctgctggcggaccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggctaagggccagcctcgggaacctcaggtttacacactgcctccaagccgggaagagatgaccaagaatcaggtgtccctgacctgcctcgtgaagggcttctacccttccgatatcgccgtcgaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtcactctccctttccccgggcgctggcggcggaggatctggcggaggcggtagcggtggtggtggatctgcccagcagaagtatcagcctctggacgagctggacaagaccctgtacgaccagttcatgctccagcagggactggaatga

SEQ ID NO: 57 - DNA, 1584 bp, synthetic construct
  misc_feature 1..1584: note = DNA sequence (DHAMDH02083016) for 2xCon4(C) fused to the C-terminus of the Heavy Chain of Bevacizumab, with linker peptide GGGGSGGGGSGGGGS
  source 1..1584: mol_type = other DNA
atgggttggtcctgtatcattcttttcctcgtcgccactgccaccggagtccactcagaagtccagttggtggagtcgggaggaggactggtgcagccaggcggctccctgcgcctgtcctgcgcggcgtccgggtacaccttcaccaactacggcatgaactgggtccgccaggcccccggaaaggggctggaatgggtcggctggatcaacacttacaccggagaacctacctacgctgccgatttcaagcggcgctttactttctcgctggacacctccaagagcaccgcctatctccaaatgaactccctgcgggccgaggataccgccgtgtactattgcgcgaagtacccccactattacggttcgtcccattggtacttcgacgtctggggccagggaactcttgtcactgtgtcctccgcatccaccaagggaccgtcagtgttccccctggccccgtcctccaaaagcactagcggaggaaccgcagccttgggatgcctcgtcaaggactactttcccgagcctgtcaccgtgtcgtggaactccggtgccctcacttcgggcgtgcacacgttcccagcggtgctgcagtccagcggactgtactcgctgtcctccgtcgtgaccgtgccttcatcgagcctggggacccagacctacatttgcaacgtgaaccacaagccctccaacaccaaagtggacaagaaggtcgaaccaaagagctgcgacaagacccacacttgcccgccgtgcccggcccctgagttgctgggtggtccatcggtgttcctgttcccgcctaagccgaaggacacactcatgatcagcaggacccccgaagtgacctgtgtggtggtcgacgtgtcacatgaagatcccgaggtcaagttcaattggtacgtggacggagtggaagtgcataatgccaagactaagccgagagaggaacagtacaactccacctaccgggtggtgtcagtgctgaccgtgctccatcaggactggctcaacgggaaggagtacaagtgcaaagtgtcgaacaaggctctccccgcccctatcgagaaaaccattagcaaggctaagggacagccgcgggagccgcaagtgtacaccctgcccccgagccgcgaagaaatgactaagaaccaagtgtccctgacctgtctcgtgaaagggttctacccgtcggacatcgctgtggagtgggagtctaatggtcaacctgagaacaactacaagactactccccctgtgctggactccgatggttcctttttcctgtactcaaagctgaccgtggacaagtccagatggcagcagggcaacgtgttcagctgctccgtgatgcatgaagcacttcacaaccactacacccagaagtccctcagcctgtctccggggaagggcggcggaggaggggcccagcaggaagagtgtgaatgggacccctggacttgtgaacacatgggcggcggcggctccggtggaggaggatccggcggagggggcagcgcgacgcaccaggaggagtgcgaatgggatccatggacttgcgaacacatgctggagtga

SEQ ID NO: 58 - DNA, 702 bp, synthetic construct
  misc_feature 1..702: note = DNA sequence (DHAMDL083016), for the light chain of Bevacizumab
  source 1..702: mol_type = other DNA
atgggttggtcctgtattatcctctttctcgtcgccactgccaccggagtgcactcagatattcagatgacccagagcccctcctcactgtccgcttccgtgggggaccgcgtgactatcacttgctcggcttcccaagatatctccaactacctgaactggtaccagcagaagcccggaaaggccccgaaagtgctcatctacttcacctcatcgctgcactcgggagtgccctcaagattttccggctccggaagcgggaccgacttcactcttaccatctcatcgttgcaaccagaggatttcgcgacctactactgtcagcagtactccacggtgccgtggaccttcggacaaggcaccaaagtggagatcaagaggactgtggcggccccgagcgtgttcattttccctccttccgacgagcagctgaaaagcggcaccgcctcggtcgtgtgcctcctgaacaacttctacccgcgggaagccaaggtccagtggaaggtcgacaacgcgctgcagagcggaaattcccaggagagcgtgaccgaacaggactccaaggacagcacctattccctgtcgtctacactgaccctgagcaaggccgactacgagaagcataaggtctacgcatgcgaagtgacccaccaaggtctttcctcccctgtgaccaagtccttcaaccggggcgaatgctga

SEQ ID NO: 59 - DNA, 822 bp, synthetic construct
  misc_feature 1..822: note = DNA sequence (LY2.55.1), for peptide L1-15 (no LE) fused to the N-terminus of the light chain of Bevacizumab
  source 1..822: mol_type = other DNA
atggcctggatgatgttgcttctcggacttctcgcgtatggatcaggggtggatagcgcgcaacagaagtaccagcctttggacgaactggacaagaccctgtacgaccagttcatgctgcaacagggagggggcggtggatccgggggcggcggctccggcggtggcggatccgacattcaaatgactcagtcgccatcgtccctctcggcatccgtgggagacagagtgaccatcacttgttccgcctcgcaagacatctccaactacctgaactggtaccagcagaagcccgggaaggcccccaaagtgctcatctactttacttcctcactgcactccggggtgccaagccgctttagcggctccggttctggaaccgatttcaccctgaccattagctcactccagccggaagatttcgctacgtactactgccagcagtattcgaccgtgccgtggactttcggacagggtaccaaagtcgagatcaagcggaccgtggccgccccgagcgtgttcattttcccgccttccgacgagcaactcaagtccggcactgcctccgtggtctgcctgctgaacaatttctacccccgcgaggctaaggtccagtggaaggtcgataacgcactgcagtccggaaacagccaagagagcgtgaccgaacaggactccaaggactcaacttactcgctgagctccaccctgaccctgtcgaaggccgactacgaaaagcacaaagtgtacgcctgcgaagtgacacatcagggcctgtcatcccctgtcaccaagtccttcaaccggggagagtgctgataa

SEQ ID NO: 60 - DNA, 828 bp, synthetic construct
  misc_feature 1..828: note = DNA sequence (LY2.55.2), for peptide L1-15 (with LE) fused to the N-terminus of the light chain of Bevacizumab
  source 1..828: mol_type = other DNA
atggcctggatgatgttgcttctcggacttctcgcgtatggatcaggggtggatagcgcgcaacagaagtaccagcctttggacgaactggacaagaccctgtacgaccagttcatgctgcaacagggactggaagggggcggtggatccgggggcggcggctccggcggtggcggatccgacattcaaatgactcagtcgccatcgtccctctcggcatccgtgggagacagagtgaccatcacttgttccgcctcgcaagacatctccaactacctgaactggtaccagcagaagcccgggaaggcccccaaagtgctcatctactttacttcctcactgcactccggggtgccaagccgctttagcggctccggttctggaaccgatttcaccctgaccattagctcactccagccggaagatttcgctacgtactactgccagcagtattcgaccgtgccgtggactttcggacagggtaccaaagtcgagatcaagcggaccgtggccgccccgagcgtgttcattttcccgccttccgacgagcaactcaagtccggcactgcctccgtggtctgcctgctgaacaatttctacccccgcgaggctaaggtccagtggaaggtcgataacgcactgcagtccggaaacagccaagagagcgtgaccgaacaggactccaaggactcaacttactcgctgagctccaccctgaccctgtcgaaggccgactacgaaaagcacaaagtgtacgcctgcgaagtgacacatcagggcctgtcatcccctgtcaccaagtccttcaaccggggagagtgctgataa

SEQ ID NO: 61 - DNA, 1539 bp, synthetic construct
  misc_feature 1..1539: note = DNA sequence (LY2.55.3), for peptide L1-15 (no LE) fused to the N-terminus of the heavy chain of Bevacizumab
  source 1..1539: mol_type = other DNA
atggcttggatgatgctgctgcttggccttctcgcatacggttccggagtcgatagcgcccaacagaagtaccagcctctggacgaactggataagaccctgtacgatcagttcatgctgcaacaggggggcggcggaggatcgggcggtggtggatccggcggcggcggatccgaagtgcagctcgtggagagcgggggcggactcgtgcagccgggaggttcgctgagattgtcctgtgccgcctccggttacacctttaccaattacgggatgaactgggtccgccaggcccccggaaagggactggaatgggtcggctggatcaacacatataccggagagcccacctacgccgcggacttcaagcggagattcaccttttcactggatacgtcaaagtcaactgcatacctccagatgaactcccttagggcggaagataccgccgtgtactactgcgccaagtacccgcactattacgggtccagccattggtacttcgacgtctggggacaggggaccctcgtgaccgtcagcagcgcctccaccaagggcccgtccgtgttccctcttgcgccgtcgtccaaaagcacttccggcggcactgccgccctgggctgcctcgtgaaggattacttcccggaaccggtcaccgtgtcgtggaactccggagccctgacttcgggtgtccacaccttccctgcggtgctgcagagctccggtctgtactccctctcttccgtggtcacggtgccctcctcatcactgggaacccagacctacatctgcaacgtgaaccacaagccctcaaacactaaggtcgacaagaaagtcgaaccgaagtcgtgcgacaagacccacacttgccctccgtgcccggctcccgagctgctggggggcccttccgtgtttttgttcccgccgaaaccaaaggacactctgatgatcagccgcactccggaagtgacctgtgtggtggtggacgtgtcccacgaggacccagaagtgaaattcaattggtacgtggatggcgtggaagtgcacaacgctaagactaagccccgcgaggaacagtacaacagcacttaccgggtggtgtcggtgctcaccgtgctgcaccaagattggctcaacgggaaggagtacaagtgcaaagtctccaacaaggccctgcccgcacctattgaaaagaccatcagcaaggccaagggacagccccgggagccccaggtctacaccctgcctccctcgcgcgaagagatgactaagaaccaagtgtccctgacctgtctggtcaagggattctatccttccgacattgccgtggaatgggagtccaacgggcagccagagaacaactacaagaccactccacctgtgctggactccgacgggtccttcttcttgtactcgaagctgaccgtggacaagtcccggtggcagcagggaaacgtgttcagctgctccgtgatgcacgaggccttgcataatcattacacccaaaagtcgctgagcttgagcccgggaaagtgataa

SEQ ID NO: 62 - DNA, 1545 bp, synthetic construct
  misc_feature 1..1545: note = DNA sequence (LY2.55.4), for peptide L1-15 (with LE) fused to the N-terminus of the heavy chain of Bevacizumab
  source 1..1545: mol_type = other DNA
atggcttggatgatgctgctgcttggccttctcgcatacggttccggagtcgatagcgcccaacagaagtaccagcctctggacgaactggataagaccctgtacgatcagttcatgctgcaacaggggcttgagggcggcggaggatcgggcggtggtggatccggcggcggcggatccgaagtgcagctcgtggagagcgggggcggactcgtgcagccgggaggttcgctgagattgtcctgtgccgcctccggttacacctttaccaattacgggatgaactgggtccgccaggcccccggaaagggactggaatgggtcggctggatcaacacatataccggagagcccacctacgccgcggacttcaagcggagattcaccttttcactggatacgtcaaagtcaactgcatacctccagatgaactcccttagggcggaagataccgccgtgtactactgcgccaagtacccgcactattacgggtccagccattggtacttcgacgtctggggacaggggaccctcgtgaccgtcagcagcgcctccaccaagggcccgtccgtgttccctcttgcgccgtcgtccaaaagcacttccggcggcactgccgccctgggctgcctcgtgaaggattacttcccggaaccggtcaccgtgtcgtggaactccggagccctgacttcgggtgtccacaccttccctgcggtgctgcagagctccggtctgtactccctctcttccgtggtcacggtgccctcctcatcactgggaacccagacctacatctgcaacgtgaaccacaagccctcaaacactaaggtcgacaagaaagtcgaaccgaagtcgtgcgacaagacccacacttgccctccgtgcccggctcccgagctgctggggggcccttccgtgtttttgttcccgccgaaaccaaaggacactctgatgatcagccgcactccggaagtgacctgtgtggtggtggacgtgtcccacgaggacccagaagtgaaattcaattggtacgtggatggcgtggaagtgcacaacgctaagactaagccccgcgaggaacagtacaacagcacttaccgggtggtgtcggtgctcaccgtgctgcaccaagattggctcaacgggaaggagtacaagtgcaaagtctccaacaaggccctgcccgcacctattgaaaagaccatcagcaaggccaagggacagccccgggagccccaggtctacaccctgcctccctcgcgcgaagagatgactaagaaccaagtgtccctgacctgtctggtcaagggattctatccttccgacattgccgtggaatgggagtccaacgggcagccagagaacaactacaagaccactccacctgtgctggactccgacgggtccttcttcttgtactcgaagctgaccgtggacaagtcccggtggcagcagggaaacgtgttcagctgctccgtgatgcacgaggccttgcataatcattacacccaaaagtcgctgagcttgagcccgggaaagtgataa

SEQ ID NO: 63 - DNA, 705 bp, synthetic construct
  misc_feature 1..705: note = DNA sequence (LY2.55.5), for the light chain of Bevacizumab
  source 1..705: mol_type = other DNA
atggcctggatgatgttgcttctcggacttctcgcgtatggatcaggggtggactccgacattcaaatgactcagtcgccatcgtccctctcggcatccgtgggagacagagtgaccatcacttgttccgcctcgcaagacatctccaactacctgaactggtaccagcagaagcccgggaaggcccccaaagtgctcatctactttacttcctcactgcactccggggtgccaagccgctttagcggctccggttctggaaccgatttcaccctgaccattagctcactccagccggaagatttcgctacgtactactgccagcagtattcgaccgtgccgtggactttcggacagggtaccaaagtcgagatcaagcggaccgtggccgccccgagcgtgttcattttcccgccttccgacgagcaactcaagtccggcactgcctccgtggtctgcctgctgaacaatttctacccccgcgaggctaaggtccagtggaaggtcgataacgcactgcagtccggaaacagccaagagagcgtgaccgaacaggactccaaggactcaacttactcgctgagctccaccctgaccctgtcgaaggccgactacgaaaagcacaaagtgtacgcctgcgaagtgacacatcagggcctgtcatcccctgtcaccaagtccttcaaccggggagagtgctgataa

SEQ ID NO: 64 - protein, 491 aa, synthetic construct
  REGION 1..491: note = 2xCon4(C) fused to the C-terminus of the VEGF Trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGAQQEECEWDPWTCEHMGSGSATGGSGSTASSGSGSATHQEECEWDPWTCEHMLE

SEQ ID NO: 65 - protein, 11 aa, synthetic construct
  REGION 1..11: note = VL-CDR1 of Ranibizumab VEGF Fab
SASQDISNYLN

SEQ ID NO: 66 - protein, 7 aa, synthetic construct
  REGION 1..7: note = VL-CDR2 of Ranibizumab VEGF Fab
FTSSLHS

SEQ ID NO: 67 - protein, 9 aa, synthetic construct
  REGION 1..9: note = VL-CDR3 of Ranibizumab VEGF Fab
QQYSTVPWT

SEQ ID NO: 68 - protein, 8 aa, synthetic construct
  REGION 1..8: note = VH-CDR1 of Ranibizumab VEGF Fab
GYDFTHYG

SEQ ID NO: 69 - protein, 8 aa, synthetic construct
  REGION 1..8: note = VH-CDR2 of Ranibizumab VEGF Fab
INTYTGEP

SEQ ID NO: 70 - protein, 18 aa, synthetic construct
  REGION 1..18: note = VH-CDR3 of Ranibizumab VEGF Fab
YCAKYPYYYGTSHWYFDV